(19) United States
(12) Patent Application Publication
(10) Pub. No.: US 2025/0243219 A1
(43) Pub. Date: Jul. 31, 2025

(54) MACROCYCLIC COMPOUNDS AND USES THEREOF

(71) Applicant: Cypralis Limited, Cambridge (GB)

(72) Inventors: Victoria Alexandra Steadman, Cambridge (GB); David Kenneth Dean, Cambridge (GB); Steven Stanway, Cambridge (GB); Antoinette Wilson, Cambridge (GB); Andrew Keats, Cambridge (GB); Michael Peel, Cambridge (GB)

(21) Appl. No.: 18/866,930

(22) PCT Filed: May 19, 2023
(86) PCT No.: PCT/GB2023/051335

§ 371 Date: Nov. 18, 2024

(30) Foreign Application Priority Data
May 19, 2022 (GB) 2207348.0

(51) Int. Cl. C07D 498/18; A61K 31/504; A61P 25/08; A61P 25/28; C07D 471/18; C07D 498/22

(52) U.S. Cl. CPC C07D 498/18; A61K 31/504; A61P 25/08; A61P 25/28; C07D 471/18; C07D 498/22

(57) ABSTRACT

The invention relates to selected macrocyclic compounds, and their use in the treatment or prevention of diseases and disorders. In particular, though not exclusively, the invention relates to the use of selected macrocyclic compounds in the treatment or prevention of diseases that are caused by (1) cell death due to various causes and (2) inflammation. Such diseases can affect all parts of the body, including the central and peripheral nervous systems, all organs and tissues, the muscles, the vasculature and the bones.

### FIELD OF THE INVENTION

The present invention relates to selected macrocyclic compounds, and their use in the treatment or prevention of diseases and disorders. In particular, though not exclusively, the invention relates to the use of selected macrocyclic compounds in the treatment or prevention of diseases that are caused by (1) cell death due to various causes and (2) inflammation. Such diseases can affect all parts of the body, including the central and peripheral nervous systems, all organs and tissues, the muscles, the vasculature and the bones. Diseases where inflammation is caused by cellular necrosis are called necroinflammatory diseases (Linkermann (Cell Death & Differentiation, https://doi.org/10.1038/s41418-018-0218-0 (23 Nov. 2018)).

Thus, the invention relates to selected macrocyclic compounds, and their use in the treatment or prevention of disorders of the central and peripheral nervous system including neurodegenerative and neuroinflammatory diseases, epilepsy, and stroke; of necroinflammatory diseases; of ischaemia and reperfusion injury; of diseases caused by reduced blood flow; of inherited mitochondrial diseases; of muscular dystrophies and myopathies.

### BACKGROUND TO THE INVENTION

Role of mitochondria in disease (1): Regulating cell death and mitochondrial permeability transition Mitochondria are not only the main source of adenosine triphosphate (ATP) in the cell, they are also master controllers of cell death, engaged in the pathogenesis of human diseases and aging. This role relies directly or indirectly on a broad range of signalling pathways (Javadov et al., Cells, 9, 1177 (2020)). Mitochondrial dysfunction can have many causes which usually converge on a common final pathway which involves Mitochondrial Permeability Transition, specifically opening of the Mitochondrial Permeability Transition Pore ‘MPTP’ (Izzo et al., Trends in Cell Biology, May, 26, 9 (2016)).

Cell death is a physiological necessity to maintain tissue homoeostasis and to eliminate harmful cells. There are three distinct and morphologically different types of cell death, that can partially overlap: Type 1 (apoptosis); type 2 (autophagy); and type 3 (necrosis) (for example, Green et al. (Cold Spring Harb. Perspect Biol., 7, a006080 (2015)). Apoptosis and autophagy are orderly processes that recycle the building materials of the cell. These processes require ATP. Apoptosis can be triggered by activation of death ligand receptors (“extrinsic pathway”) or by mitochondrial permeability transition (“intrinsic pathway”). Necrosis occurs as a consequence of irreparable cell damage and is characterized by cell swelling, plasma membrane rupture and discharge of cellular components into the blood stream. It is the intrinsic and necrotic pathways where the compounds of the present invention exert their activity.

A large body of scientific literature is about stress factors that cause the MPTP to open. These include elevated Ca²⁺ concentrations, reactive oxygen species (ROS), lack of oxygen (ischaemia, hypoxia), ATP depletion, exogenous or endogenous toxins, accumulation of unfolded or misfolded proteins (endoplasmic reticulum (ER) stress), DNA damage, radiation, or infection (Wong et al. (Methods Mol. Biol., 810, 235-242 (2012)); Rottenberg et al. (Aging Cell, Oct., 16, 5, 943-955 (2017)); Bouhamida et al. (Biology, 11, 300 (2022)); Lemasters et al. (Mol. Cell Biochem., Sep, 174, 1-2, 159-165 (1997)); Kornfeld et al. (Circulation Research, May 22, 1783 (2015)); and Chaudhari et al. (Frontiers in Cellular Neuroscience, July, 8, Article 213 (2014))). The process of aging has also been associated with a decline in mitochondrial quality and function (Sun et al., Mol. Cell., March 3, 61, 5, 654-666 (2016); and Vereczki et al., Mitochondrian, 34, 115-126 (2017)).

The MPTP was discovered in 1979. It is a multiprotein complex whose precise molecular composition is still not known. It is formed in the inner mitochondrial membrane upon exposure of a cell to stress factors such as the ones discussed above. MPTP opening leads to mitochondrial swelling, decrease in membrane potential (depolarisation) and ultimately may result in cell death by one of the three intrinsic pathways, depending on the particular cellular setting (for example, Baines, Ann. Rev. Physiol., 72, 61-80 (2010)).

Few proteins making up the MPTP have been identified with certainty, one of them being cyclophilin D. Cyclophilins are a large family of proteins with ubiquitous occurrence in all living organisms. The first cyclophilin (later called cyclophilin A) was identified as a specific binding protein of the immunosuppressant cyclosporin A (Handschumacher et al., Science, Nov 2, 226, 4674, 544-547 (1984)) and later again as a protein folding catalyst (Fischer et al, Nature, 337, 476-478 (1989)). In 1989 it was reported that the immunosuppressant cyclosporin A was a potent inhibitor of the mitochondrial inner membrane permeability transition (Broekemeier et al., J. Biol. Chem., 264, 14, 7826-7830 (1989)). This was subsequently followed by the identification of a mitochondrial cyclophilin which was called cyclophilin D (Griffiths et al., Biochem. J., 274, 611-614 (1991)). In 2002 it was reported, that mitochondrial inner membrane permeability transition was also inhibited by N-methyl-4-isoleucine cyclosporin (NIM811), a close analogue of cyclosporin A with potent binding to cyclophilins, but completely devoid of immunosuppressive activity (Waldmeier et al., Molecular Pharmacology, July, 62, 1, 22-29 (2002)). This result demonstrated that the immunosuppressive activity of cyclosporin A could be separated from the property of inhibiting mitochondrial permeability transition. Notably, inhibition of cyclophilin D by gene knockout of cyclosporin A does not provide absolute protection against cell death. Strong enough stress will still cause cell death. The role of cyclophilin D is that of a modulator of the MPTP and its inhibition renders cells more resistant to cell death. From these observations it became also clear that inhibition of cyclophilin D affects necrosis and the intrinsic mitochondrial death pathways but not the extrinsic pathways, sparing the physiologically necessary part of the cell death mechanisms.

Table 1 lists diseases in which cellular stress and mitochondrial permeability transition have been shown to play a central role in pathophysiology and where inhibition of cyclophilin D could result in a demonstrable therapeutic benefit. A particular advantage of the compounds of the present invention is their property of crossing the blood-brain barrier, which is a prerequisite for the treatment of diseases affecting the central nervous system.

TABLE 1
Diseases with demonstrated MPTP involvement

| Disease | References |
|---|---|
| Alzheimer's disease | Du H et al. (Nature Medicine, 14, 10, 1097-1105 (2008)). |
| | Guo L et al. (PLoS ONE, 8, 1, e54914 (2013)). |
| | Cheignon C et al. (Redox Biology, 450-464 (2018)). |
| Amyotropic Lateral | Martin L J (Biochim. Biophys. Acta. January, 1802, 1, 186-197 (2010)). |
| Sclerosis | Kim H J et al. (Brain, 135, 2865-2874(2012)). |
| Parkinson's disease | Ludtmann M H R et al. (Nature Communications, 9, 2293 (2018)). |
| | Venderova K et al. (Cold Spring Harb. Perspect. Med., 2, a009365 (2012)). |
| Huntington's disease | Quintanilla R A et al. (Brain Res Bull. 80, 4-5, 242-247 (2009)). |
| | Quintanilla R A et al. (Molecular Neurodegeneration, 8, 45 2013)). |
| Epilepsy | Kim D Y et al. (Ann. Neurol. 78, 1, 77-87 (2015)). |
| | Villa B R et al. (Neuroscience, Session 555 (“Targeted deletion of mitochondrial |
| | cyclophilin D mediates neuroprotection in epileptic brain” (2019)). |
| Psychiatric diseases | Kubota M. et al. (Therapeutic implications of down-regulation of cyclophilin D in |
| | bipolar disorder, International Journal of Neuropsychopharmacology, 13, 1355- |
| | 1368 (2010)). |
| Multiple sclerosis | Warne J et al. (J. Biol. Chem. Vol., 291, 9, 4356-4373 (2016)). |
| | Forte M et al. (PNAS, 104, 18, 7558-7563 (2007)). |
| X-linked | Lopez-Erauskin J et al. (Brain, 135, 3584-3598 (2012)). |
| adrenoleukodystrophy | |
| Ischemia/Reperfusion | Hepatic: Panel M., Gastroenterology, 157, 5, 15 (2019)). |
| Injury | Kidney: Leong K G et al. (Toxins (Basel), October, 1, 13, 10, 700 (2021)). |
| | Leong K G et al. (Int. J. Mol. Sci. January, 22, 1, 271 (2021)). |
| | Cardiac: Dongworth R et al., (B M J Heart, 97, e8 (2011)). |
| | Alam M R et al. (J. Mol. Cell Cardiol., January, 78, 80-89 (2015)). |
| | Ocular: Kim S Y et al. (Cell Death and Disease, 5, e1105 (2014)). |
| Acute pancreatitis | Mukherjee R et al. (Gut, August, 65, 8, 1333-1346 (2016)). |
| Aging | Vereczki V et al. (Mitochondrion, 34, 115-126, (2017)). |
| Muscular dystrophy | Reutenauer J et al. (British Journal of Pharmacology, 155, 574-584 (2008)). |
| | Merlini L. et al. (Oxid. Med. Cell Longev., 2011, 39194 (2011)). |

### Role of Mitochondria in Disease (2): Inflammation

Opening of the MPTP in response to the stress factors described above can occur in two different modes: low and/or transient levels of stress result in low conductance opening, which is characterised by a partial depolarisation of the inner mitochondrial membrane. In this state, the amount of ATP produced is diminished, but still sufficient to support certain cellular functions that require ATP. From this state the cell may either recover or undergo regulated recycling processes leading to cell death such as apoptosis or autophagy (Wacquier et al, Nature Research Scientific Reports, 10, 3924 (2020)).

High and persistent levels of stress cause complete collapse of the mitochondrial membrane potential, cessation of ATP synthesis and the cell will irreversibly commit to necrotic pathways of cell death. Necrosis involves rupture of the cell membrane and spillage of cellular components into the blood stream where they act as so-called damage-associated patterns (DAMPs) to activate various arms of the innate immune system resulting in inflammation very similar to that triggered in bacterial sepsis (Roh et al. (Immune Netw., Aug, 18, 4, e27 (2018)); Gong et al. (Nature Reviews Immunology, 20, 95-112 (2020)). Inflammation caused by tissue necrosis is called necroinflammation. One prominent member of the DAMPs released from necrotic cells is cyclophilin A which is a normally cytosolic member of the cyclophilin family. Because of its appearance in the bloodstream upon necrotic cell death, cyclophilin A has been suggested as a biomarker for necrotic cell death (Christofferson et al., Cell Death and Differentiation, 17, 1942-1943 (2010)). In circulation, cyclophilin A acts as a powerful pro-inflammatory cytokine that is involved in many inflammatory diseases, including cardiovascular diseases, viral infections, neurodegeneration, cancer, rheumatoid arthritis, sepsis, asthma, periodontitis and aging (Nigro et al., Cell Death and Disease, 4, e888 (2013)). This pro-inflammatory activity of cyclophilin A is mediated by interaction with membrane receptors that are present on many different cells throughout the body. Of these receptors, only CD147 and TREM2 (triggering receptor expressed on myeloid cells 2) have been characterised (Yurchenko et al. (Immunology, 117, 301-309 (2005)); Kon-Young et al. (The FASEB Journal., 35, e21479 (2021))) and the pro-inflammatory relevance of the interaction has been firmly established using antibodies to the respective interacting proteins (for example Geng et al. (Signal Transduction and Targeted Therapy, 6, 347 (2021)).

Importantly, the interaction of cyclophilin A with both CD147 and TREM2 occurs via the cyclosporin A binding site (Bukrinski (BioChim. Biophys. Acta Gen. Subj., Oct, 1850, 10, 2087-2095 (2015))). If this site is occupied by cyclosporin A or by compounds of the present invention, no interaction with CD147 or TREM2 can take place with the consequence that the pro-inflammatory activities of cyclophilin A via activating these receptors are blocked at their origin.

Therefore, the compounds of the present invention have two different but complementary and valuable properties: inhibition of cyclophilin D results in inhibition of mitochondrial permeability transition and prevention of cell death; and binding to the cyclosporin A binding site of cyclophilin A circulating in the bloodstream prevents it from interaction with its cognate membrane receptors CD147 and TREM2 to the effect of broad anti-inflammatory activity.

### BRIEF DESCRIPTION OF THE INVENTION

According to a first aspect of the invention, a compound of formula (I):

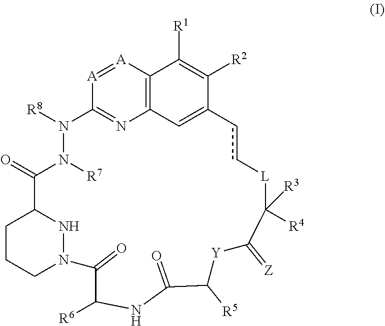

or a salt or solvate thereof, or an optical isomer, enantiomer or diastereoisomer thereof is provided, wherein;
A is independently either C or N;
R¹ and R² are independently H or methyl;
R³ and R⁴ are independently H, C₁₋₇ branched or unbranched alkyl, or can form 4-7 membered carbocyclic or heterocyclic ring in which ring heteroatoms may be O or N, optionally wherein when the heteroatom is N, N is substituted by C₁₋₇ branched or unbranched alkyl or C₁₋₄ branched or unbranched acyl, optionally wherein R³ and R⁴ are independently substituted on any C by H or phenyl;
R⁵ is H, unbranched or branched C₁₋₇-alkyl, a 4-7 membered cyclic or heterocyclic, optionally substituted by carboxy-C₅₋₇-aryl, C₅₋₇-arylamide, C₅₋₇-aryl, 5-7 membered heterocyclic in which the ring heteroatoms may be O or N, C₅₋₇-arylamido, phenyl, 5-7-membered heteroaryl, C₅₋₇-aryl aminosulfonyl, halogen, amido, or amino;
R⁶ is H, unbranched or branched C₁₋₇-alkyl or C₃₋₇-cycloalkyl, or an amido, or a carbonyl, optionally substituted by an amino, carbonyl, 5-7 membered heterocyclic or heteroaryl, or C₅₋₇-aryl;
R⁷ is H, branched or unbranched C₁₋₆-alkyl, or forms a 4-7 membered heterocyclic ring with R8;
R⁸ is H, branched or unbranched C₁₋₆-alkyl, acetyl, or forms a 4-7 membered heterocyclic ring with R7, optionally substituted by hydroxy, C₅₋₇-aryl, or carbonyl;
the dashed line is either absent or represents a single bond;
L is CH₂ or a bond;
Y is CH₂, NH, N—CH₃ or O;
C═Z is C═O or CH₂.

Thus, when the dashed line is absent, the bond is a single bond, and when the dashed line represents a single bond, the bond in total becomes a double bond.

Furthermore, when L is CH₂, the CH₂ group joins the —C(R³R⁴)— group and the sometimes double bond, and when L is a bond, there is no atom or group between the —C(R³R⁴)— group and the sometimes double bond.

In a second aspect of the invention, a pharmaceutical composition is provided, the pharmaceutical composition comprising a compound of the first aspect of the invention and a pharmaceutically acceptable carrier.

In a third aspect of the invention, a compound according to the first aspect of the invention is provided for use as a medicament.

In a fourth aspect of the invention, a compound according to the first aspect of the invention is provided, for use in treating or preventing neurodegeneration; Alzheimer's disease; Parkinson's disease; X-linked adrenoleukodystrophy; epilepsy; amyotrophic lateral sclerosis (ALS); multiple sclerosis; bipolar disorder; stroke; hearing loss; diseases caused by cellular necrosis and inflammation; acute pancreatitis; surgery-associated acute kidney injury; acute kidney injury caused by toxic drugs; hearing loss caused by ototoxic drugs; acute kidney injury caused by aminoglycoside antibiotics; bacterial sepsis with an antibiotic; sepsis without an antibiotic; diseases wherein the subject has experienced or is suffering from physical trauma or crush injury, exposure to electrical current, extreme physical exertion or activity, and temperature extremes associated with or at risk for onset of rhabdomyolysis; diseases wherein the subject has a pre-existing condition or disease that increases the subject's risk of developing a kidney condition or disease when exposed to a nephrotoxin; diseases where a pre-existing condition or disease increases the risk of chronic kidney disease optionally wherein there is a history of renal impairment or a requirement for dialysis; diseases associated with reduced blood flow; cognitive dysfunction associated with surgery or haemodialysis; or cardiac stunning associated with haemodialysis.

In a fifth aspect of the invention, a compound according to the first aspect of the invention is provided, for use in protecting a transplant organ from ischaemia and inflammation.

### BRIEF DESCRIPTION OF THE FIGURES

The invention is now illustrated with reference to the following figures which show in:

FIG. 1 propidium iodide (PI) up-take as a measure of the number of dead pancreatic acinar cells (through necrosis) as a percentage against a number of treatments with Compound (2) and cyclosporin A and controls in an in-vitro model based on pancreatic acinar cells against the toxicity of the bile acid taurolithocholic acid-3-sulphate (TLCS);

FIG. 2 the concentration (ng/ml) of Compound (2) in blood, blood plasma and in the brain 30 minutes to 7 hours after oral treatment with Compound (2) in a rodent model;

FIG. 3 the concentration (ng/ml) of Compound (2) in blood, blood plasma and in the brain 30 minutes to 7 hours after the same dosage of Compound (2) as administered orally but sub-cutaneously in a rodent model; and

FIG. 4 the mean extent of spinal cord damage (%) for experimental autoimmune encephalomyelitis (EAE) C57BL/6J mice model cohorts (the most common animal model of human multiple sclerosis (MS)) treated with just the vehicle or Compound (2) where pertussis toxin (PT) is applied to facilitate the induction of EAE.

### DETAILED DESCRIPTION OF THE INVENTION

According to a first aspect of the invention, a compound of formula (I):

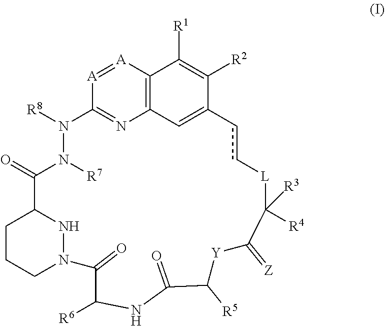

or a salt or solvate thereof, or an optical isomer, enantiomer or diastereoisomer thereof is provided, wherein;
A is independently either C or N;
R¹ and R² are independently H or methyl;
R³ and R⁴ are independently H, C₁₋₇ branched or unbranched alkyl, or can form 4-7 membered carbocyclic or heterocyclic ring in which ring heteroatoms may be O or N, optionally wherein when the heteroatom is N, N is substituted by C₁₋₇ branched or unbranched alkyl or C₁₋₄ branched or unbranched acyl, optionally wherein R³ and R⁴ are independently substituted on any C by H or phenyl;
R⁵ is H, unbranched or branched C₁₋₇-alkyl, a 4-7 membered cyclic or heterocyclic, optionally substituted by carboxy-C₅₋₇-aryl, C₅₋₇-arylamide, C₅₋₇-aryl, 5-7 membered heterocyclic in which the ring heteroatoms may be O or N, C₅₋₇-arylamido, phenyl, 5-7-membered heteroaryl, C₅₋₇-aryl aminosulfonyl, halogen, amido, or amino;
R⁶ is H, unbranched or branched C₁₋₇-alkyl or C₃₋₇-cycloalkyl, or an amido, or a carbonyl, optionally substituted by an amino, carbonyl, 5-7 membered heterocyclic or heteroaryl, or C₅₋₇-aryl;
R⁷ is H, branched or unbranched C₁₋₆-alkyl, or forms a 4-7 membered heterocyclic ring with R⁸;
R⁸ is H, branched or unbranched C₁₋₆-alkyl, acetyl, or forms a 4-7 membered heterocyclic ring with R⁷, optionally substituted by hydroxy, C₅₋₇-aryl, or carbonyl; the dashed line is either absent or represents a single bond;
L is CH₂ or a bond;
Y is CH₂, NH, N—CH₃ or O;
C═Z is C═O or CH₂.

The first aspect of the invention includes further substitutions of the substitutions set forth hereinabove with, for example, C₁₋₆-alkyl, C₁₋₆-alkoxy, —NH₂, —OH, halogens, C₁₋₃-carbonyl, naphthalene, phenyl and phenylamino groups.

Where the macrocyclic compounds of the first aspect of the invention have chiral centres, all isomers, enantiomers, diastereomers, chiral forms and racemates are included, whether in the form of isomeric mixtures or separated isomers.

To the extent that any of the macrocyclic compounds of the first aspect of the invention have acid or basic centres such as carboxylates or amino groups, then all salt forms of said compounds are included herein. In the case of pharmaceutical uses, the salt should be seen as being a pharmaceutically acceptable salt.

Pharmaceutically acceptable salts that may be mentioned include acid addition salts and base addition salts. Such salts may be formed by conventional means, for example by reaction of a free acid or a free base form of a compound with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound in the form of a salt with another counter-ion, for example using a suitable ion exchange resin.

Examples of pharmaceutically acceptable salts include acid addition salts derived from mineral acids and organic acids, and salts derived from metals such as sodium, magnesium, or preferably, potassium and calcium or organic bases such as ethanolamine, N,N-dialkylethanolamines, morpholine, etc.

Examples of acid addition salts include acid addition salts formed with acetic, 2,2-dichloroacetic, citric, lactic, mandelic, glycolic, adipic, alginic, aryl sulfonic (for example, benzenesulfonic, naphthalene-2-sulfonic, naphthalene-1,5-disulfonic and p-toluenesulfonic acids), ascorbic (e.g. L-ascorbic), L-aspartic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, gluconic (for example D-gluconic), glucuronic (for example D-glucuronic), glutamic (for example L-glutamic), α-oxoglutaric, glycolic, hippuric, hydrobromic, hydrochloric, hydriodic, isethionic, lactic (for example (+)-L-lactic and (±)-DL-lactic), lactobionic, maleic, malic (for example (−)-L-malic), (±)-DL-mandelic, metaphosphoric, methanesulfonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulfuric, tannic, tartaric (for example (+)-L-tartaric), thiocyanic, undecylenic and valeric acids.

Particular examples of salts are salts derived from mineral acids such as hydrochloric, hydrobromic, phosphoric, metaphosphoric, nitric and sulfuric acids; from organic acids, such as tartaric, acetic, citric, malic, lactic, fumaric, benzoic, glycolic, gluconic, succinic, arylsulfonic acids; and from metals such as sodium, magnesium, or preferably, potassium and calcium.

The macrocyclic compounds of the first aspect of the invention may be in the form of any crystal form, in particular any solvates of the macrocyclic compounds of the first aspect of the invention and their salts. Preferred solvates are solvates formed by the incorporation into the solid state structure (for example the crystal structure) of the macrocyclic compounds of the first aspect of the invention of molecules of a non-toxic pharmaceutically acceptable solvent (referred to below as the solvating solvent). Examples of such solvents include water, alcohols (such as ethanol, isopropanol and butanol) and dimethylsulfoxide. Solvates can be prepared by recrystallising the macrocyclic compounds of the first aspect of the invention with a solvent or mixture of solvents containing the solvating solvent. Whether or not a solvate has been formed in any given instance can be determined by subjecting crystals of the compounds to analysis using well known and standard techniques such as thermogravimetric analysis (TGA), differential scanning calorimetry (DSC) and X-ray crystallography.

The solvates can be stoichiometric or non-stoichiometric solvates. Particular solvates may be hydrates, and examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI Inc. of West Lafayette, IN, USA, 1999, ISBN 0-967-06710-3.

The macrocyclic compounds of the first aspect of the invention may contain one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹²C, ¹³C and ¹⁴C, and ¹⁶O and ¹⁸O. In an analogous manner, a reference to a particular functional group also includes within its scope isotopic variations, unless the context indicates otherwise. For example, a reference to an alkyl group such as an ethyl group also covers variations in which one or more of the hydrogen atoms in the group is in the form of a deuterium or tritium isotope, for example as in an ethyl group in which all five hydrogen atoms are in the deuterium isotopic form (a perdeuteroethyl group). The isotopes may be radioactive or non-radioactive.

Preferably, when the dashed line represents a single bond, the compound adopts a trans configuration about the single bond

Preferably, R¹ and R² are H.

In embodiments of the invention, R³ is methyl and R⁴ is methyl, benzyl, or methoxymethyl, or wherein R⁴ is methyl and R³ is methyl, benzyl, or methoxymethyl.

In other embodiments of the invention, R³ and R⁴ form together a piperidine ring, a 1,3-dioxane ring, a cyclobutyl ring, cyclopentyl ring, or a cyclohexyl ring.

Preferably, R⁵ is H, ethyl, n-propyl, isopropyl, benzyl, chlorobenzyl, methoxybenzyl, 6-(2-aminopyridyl), 3-aminopropyl, chloromethyl, beta-aminocarbonylethyl, 3-piperidinomethyl, or 4-piperidinomethyl, or hexahydropyranyl-4.

In one embodiment, R⁷ and R⁸ are joined together to form a 4 to 6 membered heterocyclic ring.

In embodiments of the invention, R⁸ is selected from the group consisting of methyl, ethyl, benzyl, acetyl and 3-hydroxypropyl.

In other embodiments of the invention, R⁶ is 4-(β-naphthyl)-aminobutyl, β-carboxyethyl, β-aminocarbonylethyl, aminocarbonylmethyl, benzyl, or pyridyl.

On some embodiments, L is a bond.

Specific examples of macrocyclic compounds according to Formula (I) include but are not limited to Compounds (1) to (82) in Table 2 including any solvate or salt thereof, and any possible optical isomer, enantiomer or diastereoisomer thereof.

TABLE 2
Specific examples of macrocyclic compounds according to Formula (I)

| | Compound |
|---|---|
| Structure | number |
| 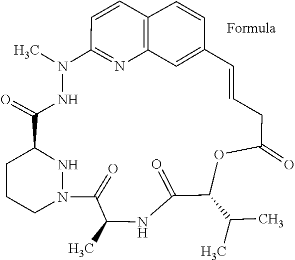 [SMILES: CC(C)[C@H]1OC(=O)C/C=C/c2ccc3ccc(nc3c2)N(C)NC(=O)[C@@H]2CCCN(N2)C(=O)[C@H](C)NC1=O] | 1 |
| 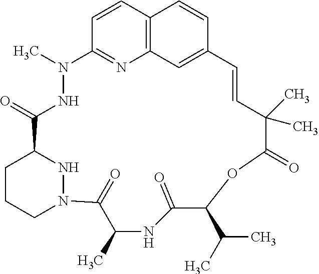 [SMILES: CC(C)[C@@H]1OC(=O)C(C)(C)/C=C/c2ccc3ccc(nc3c2)N(C)NC(=O)[C@@H]2CCCN(N2)C(=O)[C@H](C)NC1=O] | 2 |
| 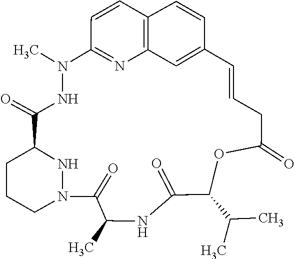 [SMILES: CC(C)[C@H]1OC(=O)C/C=C/c2ccc3ccc(nc3c2)N(C)NC(=O)[C@@H]2CCCN(N2)C(=O)[C@H](C)NC1=O] | 3 |
| 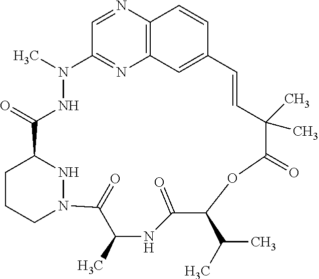 [SMILES: CC(C)[C@@H]1OC(=O)C(C)(C)/C=C/c2ccc3ncc(nc3c2)N(C)NC(=O)[C@@H]2CCCN(N2)C(=O)[C@H](C)NC1=O] | 4 |
| 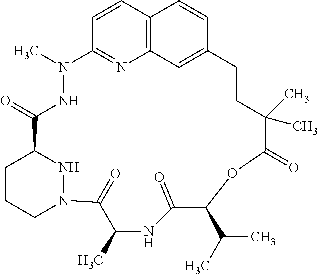 [SMILES: CC(C)[C@@H]1OC(=O)C(C)(C)CCc2ccc3ccc(nc3c2)N(C)NC(=O)[C@@H]2CCCN(N2)C(=O)[C@H](C)NC1=O] | 5 |
| 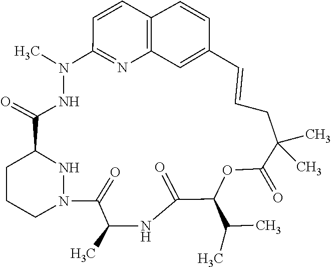 [SMILES: CC(C)[C@@H]1OC(=O)C(C)(C)C/C=C/c2ccc3ccc(nc3c2)N(C)NC(=O)[C@@H]2CCCN(N2)C(=O)[C@H](C)NC1=O] | 6 |
| 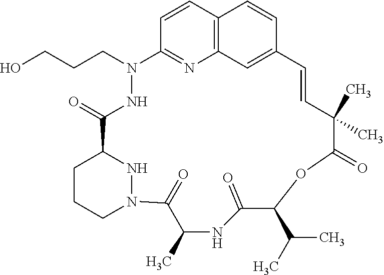 | 7 |
| 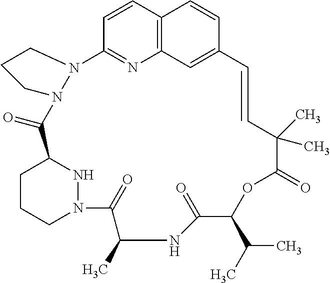 [SMILES: CC(C)[C@@H]1OC(=O)C(C)(C)/C=C/c2ccc3ccc(nc3c2)N2CCCN2C(=O)[C@@H]2CCCN(N2)C(=O)[C@H](C)NC1=O] | 8 |
| 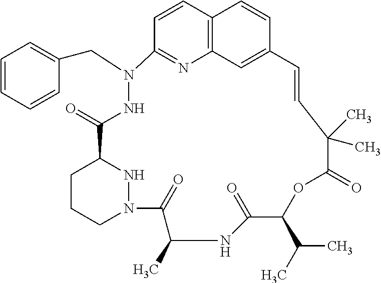 [SMILES: CC(C)[C@@H]1OC(=O)C(C)(C)/C=C/c2ccc3ccc(nc3c2)N(Cc2ccccc2)NC(=O)[C@@H]2CCCN(N2)C(=O)[C@H](C)NC1=O] | 9 |
| 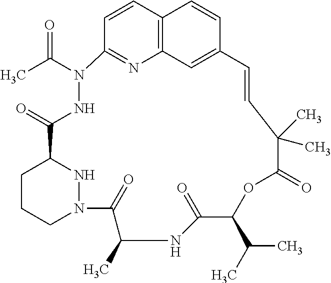 [SMILES: CC(=O)N1NC(=O)[C@@H]2CCCN(N2)C(=O)[C@H](C)NC(=O)[C@H](C(C)C)OC(=O)C(C)(C)/C=C/c2ccc3ccc1nc3c2] | 10 |
| 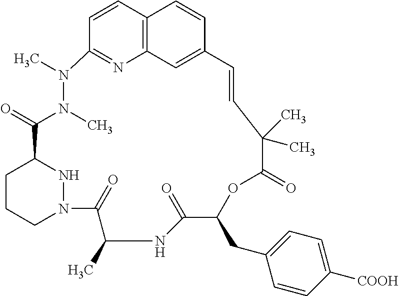 [SMILES: C[C@@H]1NC(=O)[C@H](Cc2ccc(C(=O)O)cc2)OC(=O)C(C)(C)/C=C/c2ccc3ccc(nc3c2)N(C)N(C)C(=O)[C@@H]2CCCN(N2)C1=O] | 11 |
| 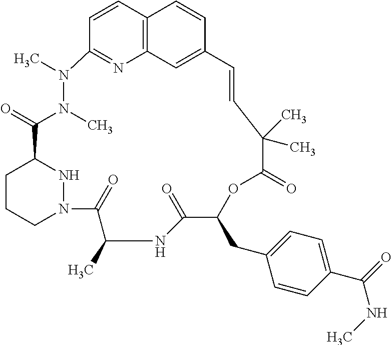 [SMILES: CNC(=O)c1ccc(C[C@@H]2OC(=O)C(C)(C)/C=C/c3ccc4ccc(nc4c3)N(C)N(C)C(=O)[C@@H]3CCCN(N3)C(=O)[C@H](C)NC2=O)cc1] | 12 |
| 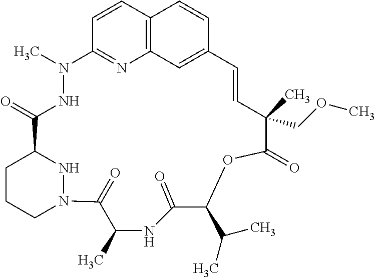 [SMILES: COC[C@@]1(C)/C=C/c2ccc3ccc(nc3c2)N(C)NC(=O)[C@@H]2CCCN(N2)C(=O)[C@H](C)NC(=O)[C@H](C(C)C)OC1=O] | 13 |
| 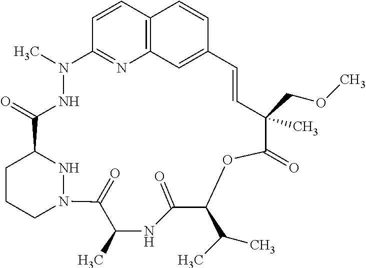 [SMILES: COC[C@]1(C)/C=C/c2ccc3ccc(nc3c2)N(C)NC(=O)[C@@H]2CCCN(N2)C(=O)[C@H](C)NC(=O)[C@H](C(C)C)OC1=O] | 14 |
| 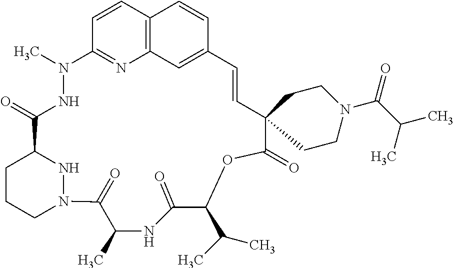 | 15 |
| 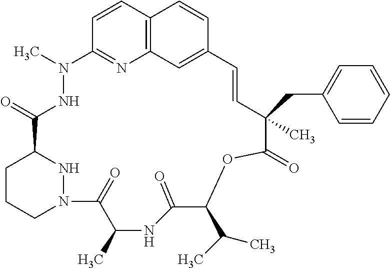 [SMILES: CC(C)[C@@H]1OC(=O)[C@](C)(Cc2ccccc2)/C=C/c2ccc3ccc(nc3c2)N(C)NC(=O)[C@@H]2CCCN(N2)C(=O)[C@H](C)NC1=O] | 16 |
| 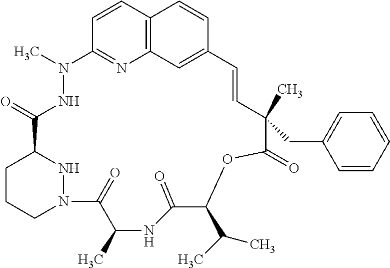 [SMILES: CC(C)[C@@H]1OC(=O)[C@@](C)(Cc2ccccc2)/C=C/c2ccc3ccc(nc3c2)N(C)NC(=O)[C@@H]2CCCN(N2)C(=O)[C@H](C)NC1=O] | 17 |
| 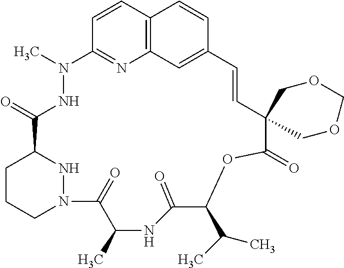 | 18 |
| 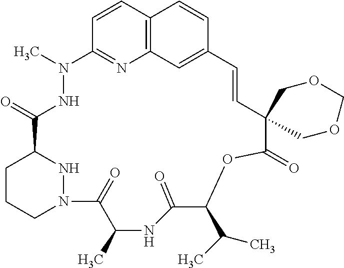 | 19 |
| 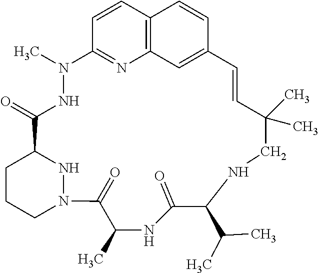 [SMILES: CC(C)[C@@H]1NCC(C)(C)/C=C/c2ccc3ccc(nc3c2)N(C)NC(=O)[C@@H]2CCCN(N2)C(=O)[C@H](C)NC1=O] | 20 |
| 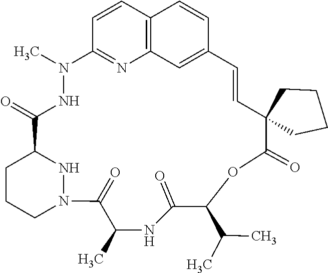 | 21 |
| 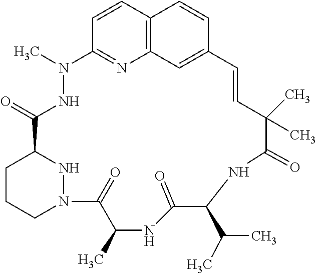 [SMILES: CC(C)[C@@H]1NC(=O)C(C)(C)/C=C/c2ccc3ccc(nc3c2)N(C)NC(=O)[C@@H]2CCCN(N2)C(=O)[C@H](C)NC1=O] | 22 |
| 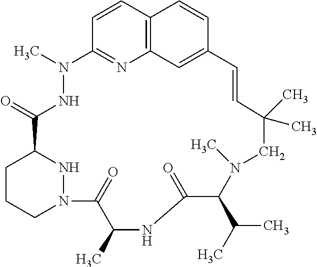 [SMILES: CC(C)[C@H]1C(=O)N[C@@H](C)C(=O)N2CCC[C@H](N2)C(=O)NN(C)c2ccc3ccc(cc3n2)/C=C/C(C)(C)CN1C] | 23 |
| 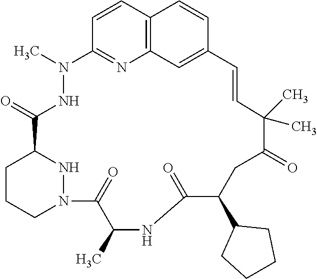 [SMILES: C[C@@H]1NC(=O)[C@H](C2CCCC2)CC(=O)C(C)(C)/C=C/c2ccc3ccc(nc3c2)N(C)NC(=O)[C@@H]2CCCN(N2)C1=O] | 24 |
| 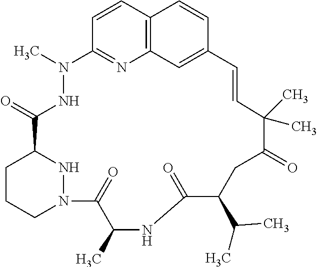 [SMILES: CC(C)[C@@H]1CC(=O)C(C)(C)/C=C/c2ccc3ccc(nc3c2)N(C)NC(=O)[C@@H]2CCCN(N2)C(=O)[C@H](C)NC1=O] | 25 |
| 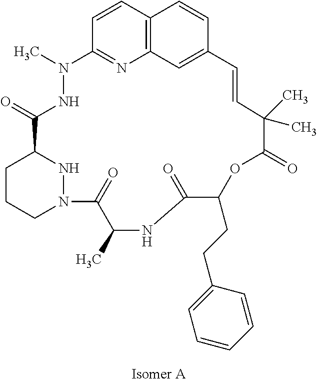 [SMILES: C[C@@H]1NC(=O)C(CCc2ccccc2)OC(=O)C(C)(C)/C=C/c2ccc3ccc(nc3c2)N(C)NC(=O)[C@@H]2CCCN(N2)C1=O] | 26 |
| 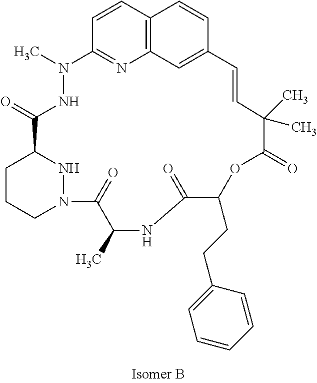 [SMILES: C[C@@H]1NC(=O)C(CCc2ccccc2)OC(=O)C(C)(C)/C=C/c2ccc3ccc(nc3c2)N(C)NC(=O)[C@@H]2CCCN(N2)C1=O] | 27 |
| 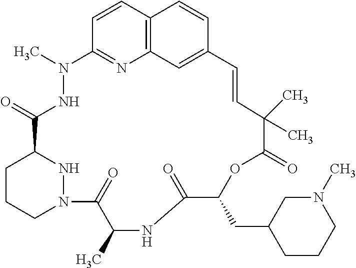 [SMILES: C[C@@H]1NC(=O)[C@@H](CC2CCCN(C)C2)OC(=O)C(C)(C)/C=C/c2ccc3ccc(nc3c2)N(C)NC(=O)[C@@H]2CCCN(N2)C1=O] | 28 |
| 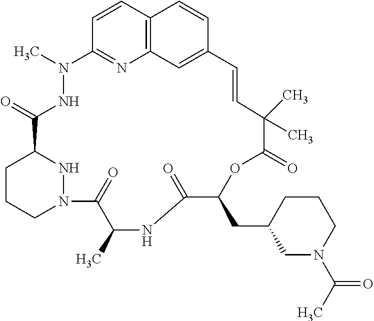 [SMILES: CC(=O)N1CCC[C@@H](C[C@@H]2OC(=O)C(C)(C)/C=C/c3ccc4ccc(nc4c3)N(C)NC(=O)[C@@H]3CCCN(N3)C(=O)[C@H](C)NC2=O)C1] | 29 |
| 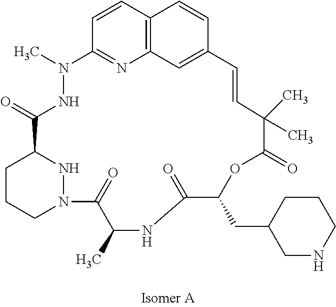 [SMILES: C[C@@H]1NC(=O)[C@@H](CC2CCCNC2)OC(=O)C(C)(C)/C=C/c2ccc3ccc(nc3c2)N(C)NC(=O)[C@@H]2CCCN(N2)C1=O] | 30 |
| 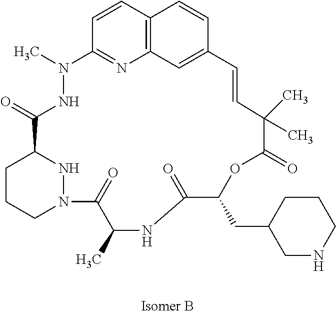 [SMILES: C[C@@H]1NC(=O)[C@@H](CC2CCCNC2)OC(=O)C(C)(C)/C=C/c2ccc3ccc(nc3c2)N(C)NC(=O)[C@@H]2CCCN(N2)C1=O] | 31 |
| 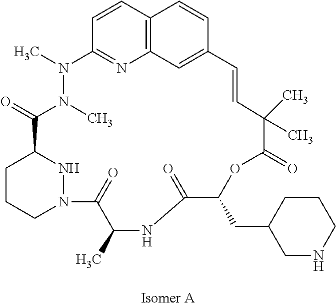 [SMILES: C[C@@H]1NC(=O)[C@@H](CC2CCCNC2)OC(=O)C(C)(C)/C=C/c2ccc3ccc(nc3c2)N(C)N(C)C(=O)[C@@H]2CCCN(N2)C1=O] | 32 |
| 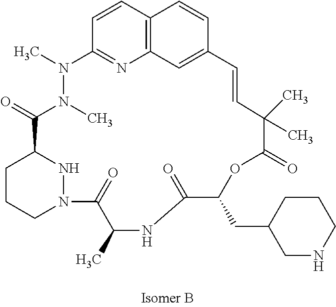 [SMILES: C[C@@H]1NC(=O)[C@@H](CC2CCCNC2)OC(=O)C(C)(C)/C=C/c2ccc3ccc(nc3c2)N(C)N(C)C(=O)[C@@H]2CCCN(N2)C1=O] | 33 |
| 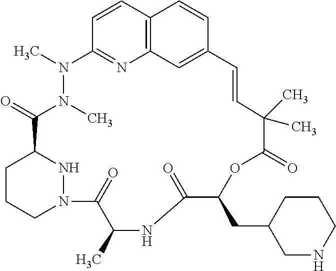 [SMILES: C[C@@H]1NC(=O)[C@H](CC2CCCNC2)OC(=O)C(C)(C)/C=C/c2ccc3ccc(nc3c2)N(C)N(C)C(=O)[C@@H]2CCCN(N2)C1=O] | 34 |
| 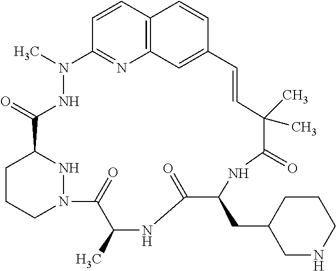 [SMILES: C[C@@H]1NC(=O)[C@H](CC2CCCNC2)NC(=O)C(C)(C)/C=C/c2ccc3ccc(nc3c2)N(C)NC(=O)[C@@H]2CCCN(N2)C1=O] | 35 |
| 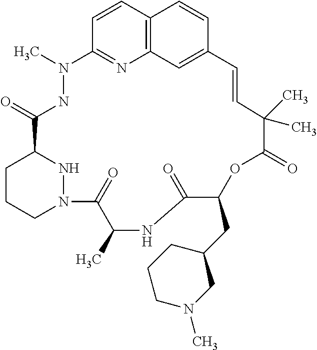 [SMILES: C[C@@H]1NC(=O)[C@H](C[C@@H]2CCCN(C)C2)OC(=O)C(C)(C)/C=C/c2ccc3ccc(nc3c2)N(C)NC(=O)[C@@H]2CCCN(N2)C1=O] | 36 |
| 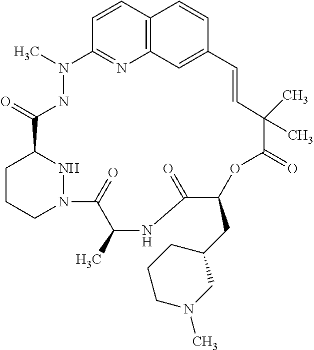 [SMILES: C[C@@H]1NC(=O)[C@H](C[C@H]2CCCN(C)C2)OC(=O)C(C)(C)/C=C/c2ccc3ccc(nc3c2)N(C)NC(=O)[C@@H]2CCCN(N2)C1=O] | 37 |
| 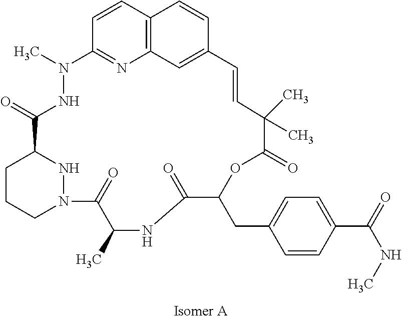 [SMILES: CNC(=O)c1ccc(CC2OC(=O)C(C)(C)/C=C/c3ccc4ccc(nc4c3)N(C)NC(=O)[C@@H]3CCCN(N3)C(=O)[C@H](C)NC2=O)cc1] | 38 |
| 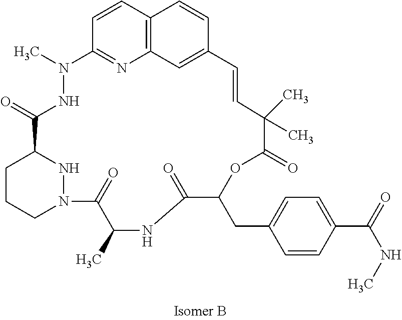 [SMILES: CNC(=O)c1ccc(CC2OC(=O)C(C)(C)/C=C/c3ccc4ccc(nc4c3)N(C)NC(=O)[C@@H]3CCCN(N3)C(=O)[C@H](C)NC2=O)cc1] | 39 |
| 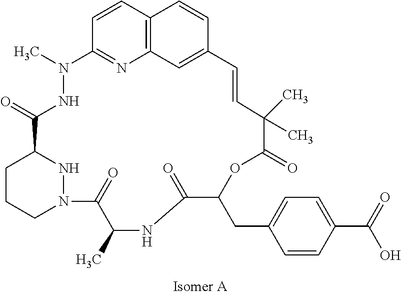 [SMILES: C[C@@H]1NC(=O)C(Cc2ccc(C(=O)O)cc2)OC(=O)C(C)(C)/C=C/c2ccc3ccc(nc3c2)N(C)NC(=O)[C@@H]2CCCN(N2)C1=O] | 40 |
| 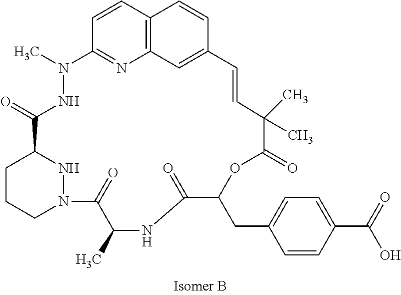 [SMILES: C[C@@H]1NC(=O)C(Cc2ccc(C(=O)O)cc2)OC(=O)C(C)(C)/C=C/c2ccc3ccc(nc3c2)N(C)NC(=O)[C@@H]2CCCN(N2)C1=O] | 41 |
| 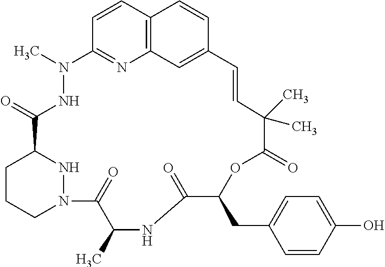 [SMILES: C[C@@H]1NC(=O)[C@H](Cc2ccc(O)cc2)OC(=O)C(C)(C)/C=C/c2ccc3ccc(nc3c2)N(C)NC(=O)[C@@H]2CCCN(N2)C1=O] | 42 |
| 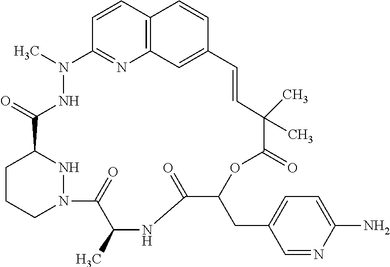 [SMILES: C[C@@H]1NC(=O)C(Cc2ccc(N)nc2)OC(=O)C(C)(C)/C=C/c2ccc3ccc(nc3c2)N(C)NC(=O)[C@@H]2CCCN(N2)C1=O] | 43 |
| 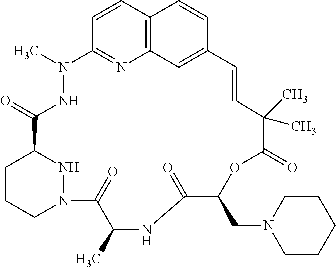 [SMILES: C[C@@H]1NC(=O)[C@H](CN2CCCCC2)OC(=O)C(C)(C)/C=C/c2ccc3ccc(nc3c2)N(C)NC(=O)[C@@H]2CCCN(N2)C1=O] | 44 |
| 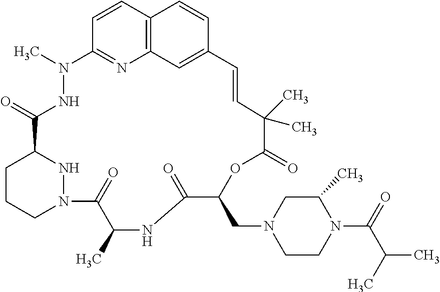 [SMILES: CC(C)C(=O)N1CCN(C[C@@H]2OC(=O)C(C)(C)/C=C/c3ccc4ccc(nc4c3)N(C)NC(=O)[C@@H]3CCCN(N3)C(=O)[C@H](C)NC2=O)C[C@@H]1C] | 45 |
| 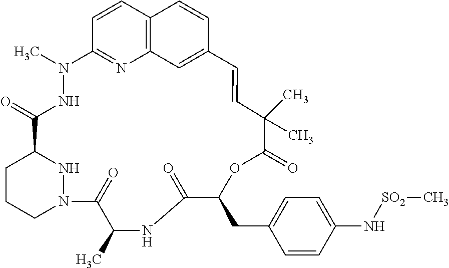 | 46 |
| 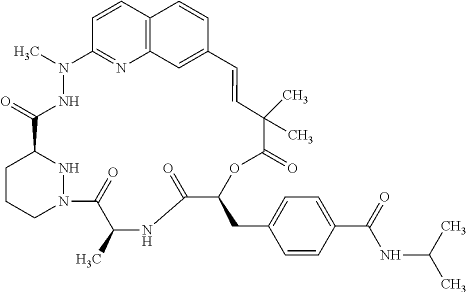 [SMILES: CC(C)NC(=O)c1ccc(C[C@@H]2OC(=O)C(C)(C)/C=C/c3ccc4ccc(nc4c3)N(C)NC(=O)[C@@H]3CCCN(N3)C(=O)[C@H](C)NC2=O)cc1] | 47 |
| 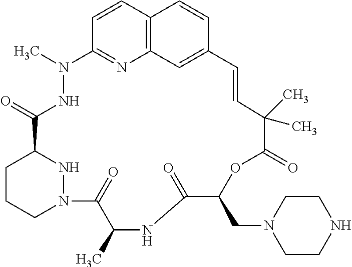 [SMILES: C[C@@H]1NC(=O)[C@H](CN2CCNCC2)OC(=O)C(C)(C)/C=C/c2ccc3ccc(nc3c2)N(C)NC(=O)[C@@H]2CCCN(N2)C1=O] | 48 |
| 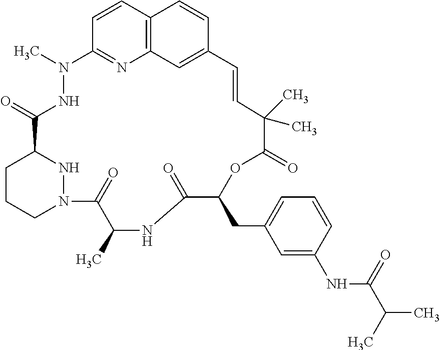 [SMILES: CC(C)C(=O)Nc1cccc(C[C@@H]2OC(=O)C(C)(C)/C=C/c3ccc4ccc(nc4c3)N(C)NC(=O)[C@@H]3CCCN(N3)C(=O)[C@H](C)NC2=O)c1] | 49 |
| 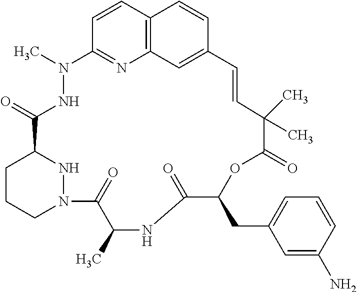 [SMILES: C[C@@H]1NC(=O)[C@H](Cc2cccc(N)c2)OC(=O)C(C)(C)/C=C/c2ccc3ccc(nc3c2)N(C)NC(=O)[C@@H]2CCCN(N2)C1=O] | 50 |
| 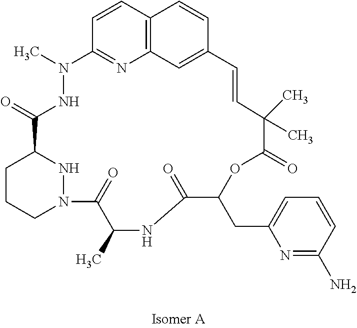 [SMILES: C[C@@H]1NC(=O)C(Cc2cccc(N)n2)OC(=O)C(C)(C)/C=C/c2ccc3ccc(nc3c2)N(C)NC(=O)[C@@H]2CCCN(N2)C1=O] | 51 |
| 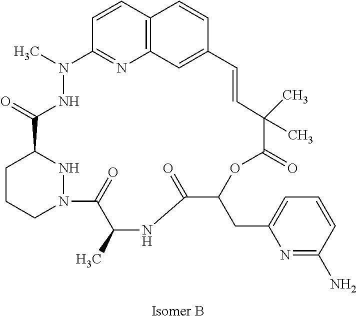 [SMILES: C[C@@H]1NC(=O)C(Cc2cccc(N)n2)OC(=O)C(C)(C)/C=C/c2ccc3ccc(nc3c2)N(C)NC(=O)[C@@H]2CCCN(N2)C1=O] | 52 |
| 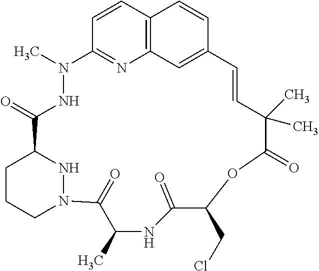 [SMILES: C[C@@H]1NC(=O)[C@H](CCl)OC(=O)C(C)(C)/C=C/c2ccc3ccc(nc3c2)N(C)NC(=O)[C@@H]2CCCN(N2)C1=O] | 53 |
| 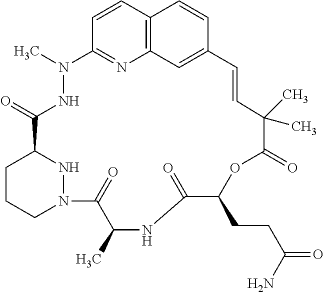 [SMILES: C[C@@H]1NC(=O)[C@H](CCC(N)=O)OC(=O)C(C)(C)/C=C/c2ccc3ccc(nc3c2)N(C)NC(=O)[C@@H]2CCCN(N2)C1=O] | 54 |
| 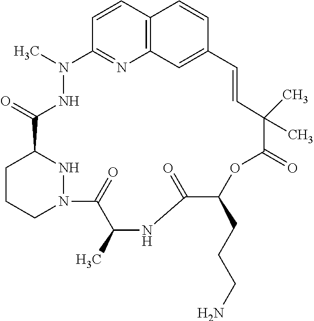 [SMILES: C[C@@H]1NC(=O)[C@H](CCCN)OC(=O)C(C)(C)/C=C/c2ccc3ccc(nc3c2)N(C)NC(=O)[C@@H]2CCCN(N2)C1=O] | 55 |
| 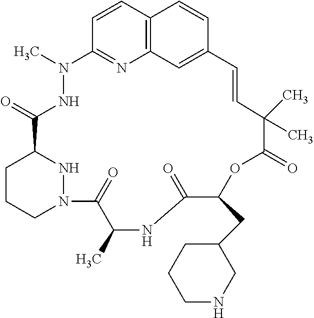 [SMILES: C[C@@H]1NC(=O)[C@H](CC2CCCNC2)OC(=O)C(C)(C)/C=C/c2ccc3ccc(nc3c2)N(C)NC(=O)[C@@H]2CCCN(N2)C1=O] | 56 |
| 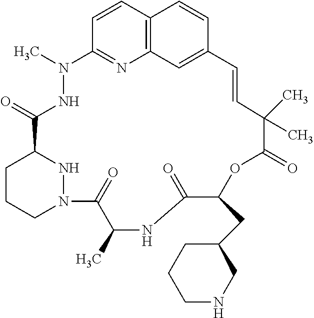 [SMILES: C[C@@H]1NC(=O)[C@H](C[C@@H]2CCCNC2)OC(=O)C(C)(C)/C=C/c2ccc3ccc(nc3c2)N(C)NC(=O)[C@@H]2CCCN(N2)C1=O] | 57 |
| 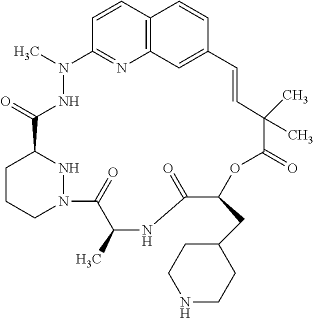 [SMILES: C[C@@H]1NC(=O)[C@H](CC2CCNCC2)OC(=O)C(C)(C)/C=C/c2ccc3ccc(nc3c2)N(C)NC(=O)[C@@H]2CCCN(N2)C1=O] | 58 |
| 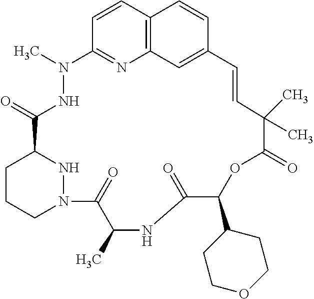 [SMILES: C[C@@H]1NC(=O)[C@H](C2CCOCC2)OC(=O)C(C)(C)/C=C/c2ccc3ccc(nc3c2)N(C)NC(=O)[C@@H]2CCCN(N2)C1=O] | 59 |
| 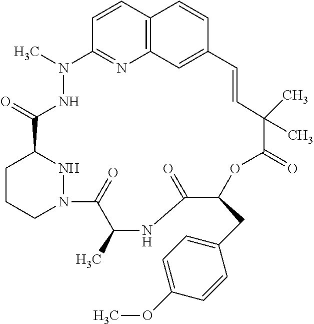 [SMILES: COc1ccc(C[C@@H]2OC(=O)C(C)(C)/C=C/c3ccc4ccc(nc4c3)N(C)NC(=O)[C@@H]3CCCN(N3)C(=O)[C@H](C)NC2=O)cc1] | 60 |
| 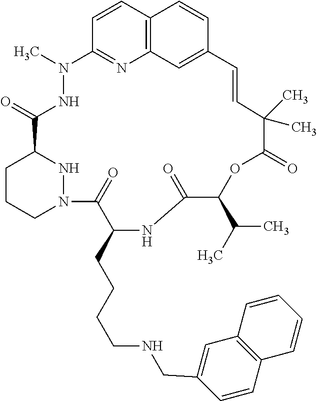 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCCCNCc2ccc3ccccc3c2)NC1=O] | 61 |
| 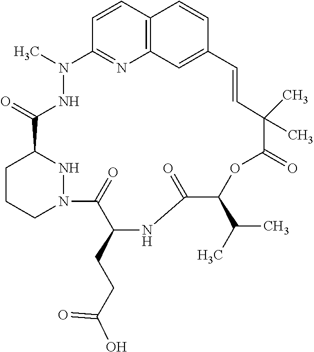 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)O)NC1=O] | 62 |
| 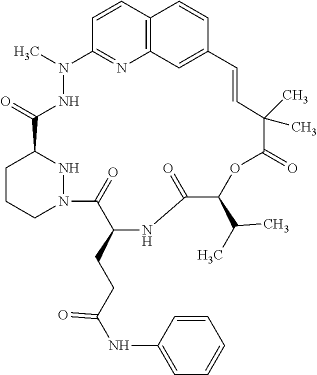 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)Nc2ccccc2)NC1=O] | 63 |
| 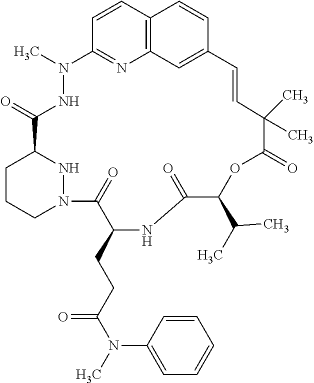 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)N(C)c2ccccc2)NC1=O] | 64 |
| 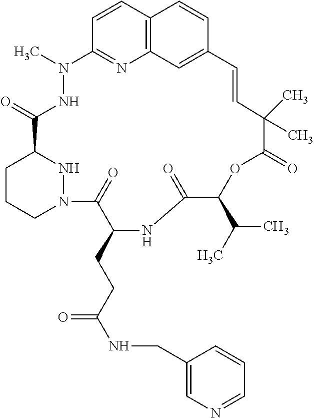 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)NCc2cccnc2)NC1=O] | 65 |
| 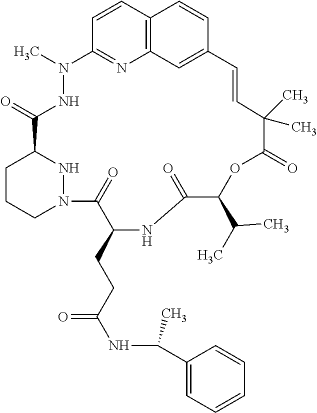 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)N[C@H](C)c2ccccc2)NC1=O] | 66 |
| 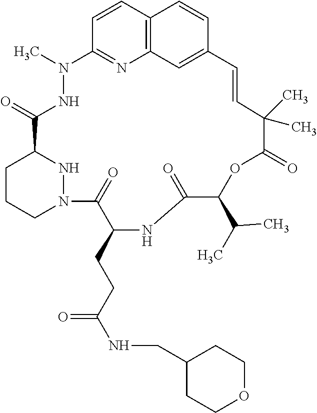 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)NCC2CCOCC2)NC1=O] | 67 |
| 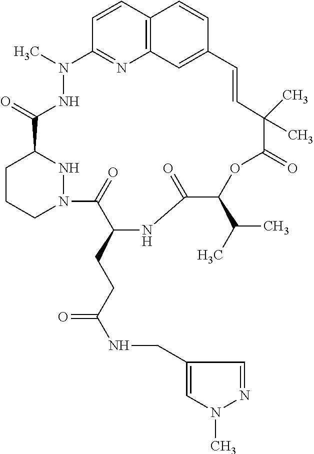 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)NCc2cnn(C)c2)NC1=O] | 68 |
| 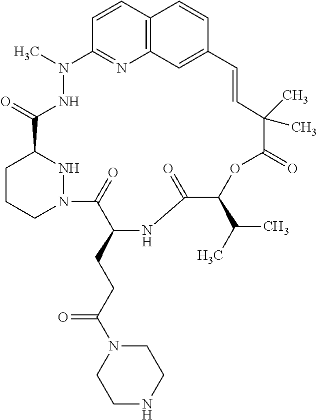 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)N2CCNCC2)NC1=O] | 69 |
| 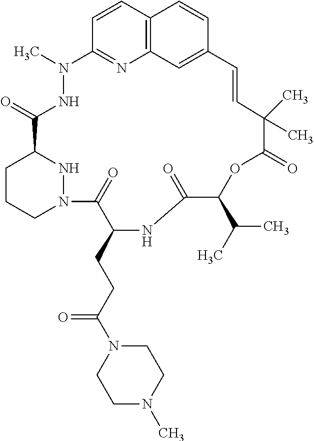 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)N2CCN(C)CC2)NC1=O] | 70 |
| 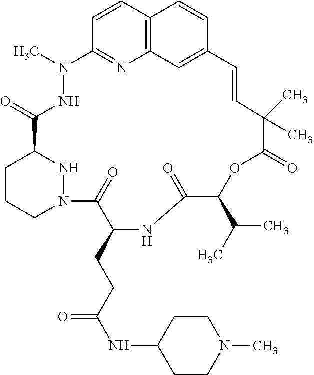 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)NC2CCN(C)CC2)NC1=O] | 71 |
| 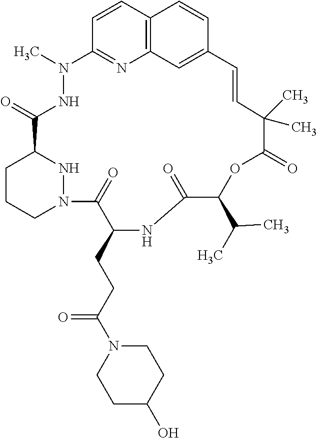 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)N2CCC(O)CC2)NC1=O] | 72 |
| 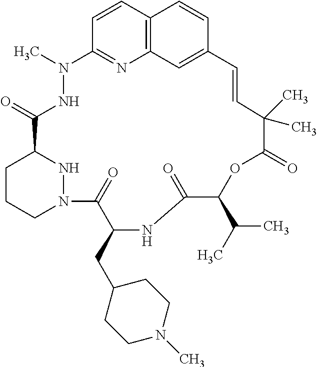 [SMILES: CC(C)[C@@H]1OC(=O)C(C)(C)/C=C/c2ccc3ccc(nc3c2)N(C)NC(=O)[C@@H]2CCCN(N2)C(=O)[C@H](CC2CCN(C)CC2)NC1=O] | 73 |
| 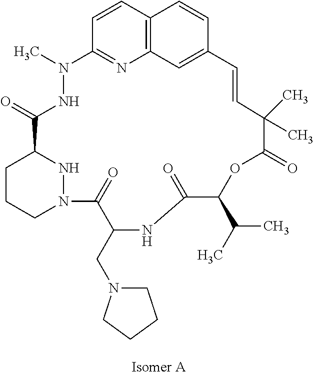 [SMILES: CC(C)[C@@H]1OC(=O)C(C)(C)/C=C/c2ccc3ccc(nc3c2)N(C)NC(=O)[C@@H]2CCCN(N2)C(=O)C(CN2CCCC2)NC1=O] | 74 |
| 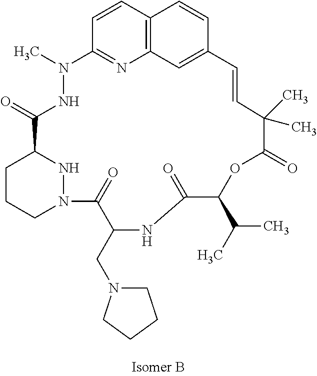 [SMILES: CC(C)[C@@H]1OC(=O)C(C)(C)/C=C/c2ccc3ccc(nc3c2)N(C)NC(=O)[C@@H]2CCCN(N2)C(=O)C(CN2CCCC2)NC1=O] | 75 |
| 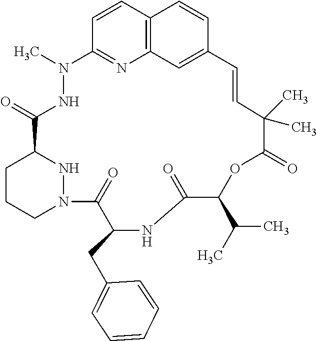 [SMILES: CC(C)[C@@H]1OC(=O)C(C)(C)/C=C/c2ccc3ccc(nc3c2)N(C)NC(=O)[C@@H]2CCCN(N2)C(=O)[C@H](Cc2ccccc2)NC1=O] | 76 |
| 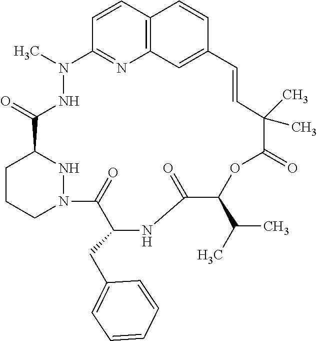 [SMILES: CC(C)[C@@H]1OC(=O)C(C)(C)/C=C/c2ccc3ccc(nc3c2)N(C)NC(=O)[C@@H]2CCCN(N2)C(=O)[C@@H](Cc2ccccc2)NC1=O] | 77 |
| 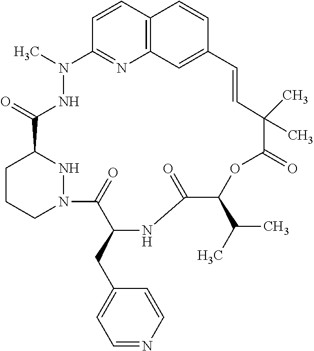 [SMILES: CC(C)[C@@H]1OC(=O)C(C)(C)/C=C/c2ccc3ccc(nc3c2)N(C)NC(=O)[C@@H]2CCCN(N2)C(=O)[C@H](Cc2ccncc2)NC1=O] | 78 |
| 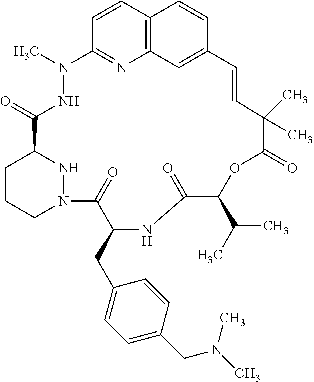 [SMILES: CC(C)[C@@H]1OC(=O)C(C)(C)/C=C/c2ccc3ccc(nc3c2)N(C)NC(=O)[C@@H]2CCCN(N2)C(=O)[C@H](Cc2ccc(CN(C)C)cc2)NC1=O] | 79 |
| 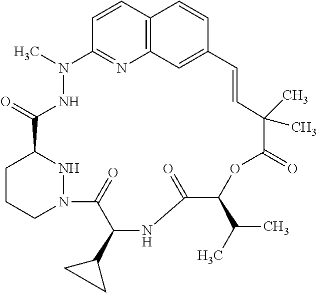 [SMILES: CC(C)[C@@H]1OC(=O)C(C)(C)/C=C/c2ccc3ccc(nc3c2)N(C)NC(=O)[C@@H]2CCCN(N2)C(=O)[C@H](C2CC2)NC1=O] | 80 |
| 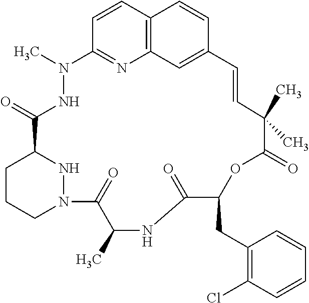 | 81 |
| 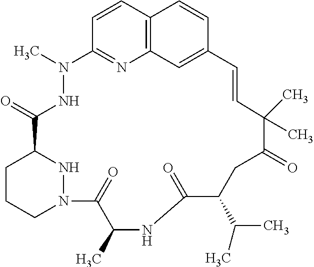 [SMILES: CC(C)[C@H]1CC(=O)C(C)(C)/C=C/c2ccc3ccc(nc3c2)N(C)NC(=O)[C@@H]2CCCN(N2)C(=O)[C@H](C)NC1=O] | 82 |

In a second aspect of the invention, a pharmaceutical composition is provided, the pharmaceutical composition comprising a compound of the first aspect of the invention and a pharmaceutically acceptable carrier.

While it is possible for the macrocyclic compounds of the first aspect of the invention to be administered alone it may be preferable to present them as a pharmaceutical composition. The pharmaceutical compositions of the second aspect of the invention, both for veterinary and for human use, comprise at least one macrocyclic compound of the first aspect of the invention as one active agent, together with one or more acceptable carriers and optionally other therapeutic ingredients. The carrier(s) must be “acceptable” in the sense of being compatible with the other ingredients of the composition and physiologically innocuous to the recipient thereof.

The term “pharmaceutical composition” in the context of this invention means a composition comprising macrocyclic compound of the first aspect of the invention as active agent and comprising additionally one or more pharmaceutically acceptable carriers. The composition may further contain ingredients selected from, for example, diluents, adjuvants, excipients, vehicles, preserving agents, fillers, disintegrating agents, wetting agents, emulsifying agents, suspending agents including liposomes or nanoparticulates, sweetening agents, flavouring agents, perfuming agents, antibacterial agents, antifungal agents, lubricating agents and dispersing agents, depending on the nature of the mode of administration and dosage forms. The compositions may take the form, for example, of tablets, dragees, powders, elixirs, syrups, liquid preparations including suspensions, sprays, inhalants, tablets, lozenges, emulsions, solutions, cachets, granules, capsules and suppositories, as well as liquid preparations for injections, including liposome preparations.

One or more macrocyclic compounds of the first aspect of the invention may be administered by any route appropriate to the condition to be treated. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with, for example, the condition of the recipient.

The pharmaceutical compositions of the second aspect of the invention include but are not limited to those suitable for the administration routes described herein. The compositions may conveniently be presented in unit dosage form and may be prepared by any of the methods well known in the art of pharmacy. Techniques and formulations generally are found in Remington's Pharmaceutical Sciences (Mack Publishing Co., Easton, Pa.), herein incorporated by reference in its entirety. Such methods include the step of bringing into association the active ingredient with the carrier, which constitutes one or more accessory ingredients. In general the compositions are prepared by uniformly and intimately bringing into association the macrocyclic compound of the first aspect of the invention as active agent with liquid carriers or finely divided solid carriers or both, and then, if necessary, shaping the compositions.

Excipients include ascorbic acid and other antioxidants, chelating agents such as EDTA, carbohydrates such as dextrin, hydroxyalkylcellulose, hydroxyalkylmethylcellulose, stearic acid and the like.

The pH of the formulations ranges from about 3 to about 11, but is ordinarily about 7 to 10.

The pharmaceutical compositions of the second aspect of the invention suitable for oral administration, each containing a predetermined amount of the active agent, may be presented as discrete units such as capsules, cachets or tablets; as a powder or granules; as a solution or a suspension in an aqueous or non-aqueous liquid; or as an oil-in-water liquid emulsion or a water-in-oil liquid emulsion. The active agent may also be administered as a bolus, electuary or paste.

Tablets may optionally be coated or scored and optionally are formulated so as to provide slow or controlled release of the active ingredient. Tablets containing the active agent in admixture with non-toxic pharmaceutically acceptable excipient which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc.

Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Pharmaceutical compositions of the second aspect of the invention for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

For administration to the eye or other external tissues, for example mouth and skin, the pharmaceutical compositions of the second aspect of the invention are preferably applied as a topical ointment or cream containing the active ingredient(s) in an amount of, for example, 0.075 to 20% w/w (including active ingredient(s) in a range between 0.1 and 20% in increments of 0.1% w/w such as 0.6% w/w, 0.7% w/w, etc.), preferably 0.2 to 15% w/w and most preferably 0.5 to 10% w/w.

When formulated in an ointment, the active agent may be employed with either a paraffinic or a water-miscible ointment base. Alternatively, the active agent may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The (topical) pharmaceutical compositions of the second aspect of the invention may desirably include a compound which enhances absorption or penetration of the active agent through the skin or other affected areas. Examples of such dermal penetration enhancers include dimethyl sulphoxide and related analogues.

The oily phase of the emulsions of the (topical) pharmaceutical compositions of the second aspect of the invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of cream formulations.

Emulgents and emulsion stabilizers suitable for use in the pharmaceutical compositions of the second aspect of the invention include Tween® 60, Span® 80, cetostearyl alcohol, benzyl alcohol, myristyl alcohol, glyceryl mono-stearate and sodium lauryl sulfate.

The choice of suitable oils or fats for the pharmaceutical compositions is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical compositions of the second aspect of the invention in the form of aqueous suspensions contain the macrocyclic compounds of the first aspect of the invention as active agents in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally occurring phosphatide (for example lecithin), a condensation product of an alkylene oxide with a fatty acid (for example polyoxyethylene stearate), a condensation product of ethylene oxide with a long chain aliphatic alcohol (for example heptadecaethyleneoxycetanol), a condensation product of ethylene oxide with a partial ester derived from a fatty acid and a hexitol anhydride (for example polyoxyethylene sorbitan monooleate). The aqueous suspension may also contain one or more preservatives such as ethyl or n-propyl p-hydroxy-benzoate, one or more colouring agents, one or more flavouring agents and one or more sweetening agents, such as sucrose or saccharin.

Pharmaceutical compositions of the second aspect of the invention in the form of oil suspensions may be formulated by suspending the macrocylic compound of the first aspect of the invention as active agent in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth herein, and flavouring agents may be added to provide a palatable oral preparation. These compositions may be preserved by the addition of an antioxidant such as ascorbic acid.

Pharmaceutical compositions of the second aspect of the invention in the form of dispersible powders and granules suitable for preparation of an aqueous suspension by the addition of water provide the active agent in admixture with a dispersing or wetting agent, a suspending agent, and one or more preservatives. Suitable dispersing or wetting agents and suspending agents are exemplified by those disclosed above. Additional excipients, for example sweetening, flavouring and colouring agents, may also be present.

Pharmaceutical compositions of the second aspect of the invention may also be in the form of oil-in-water emulsions. The oily phase may be a vegetable oil, such as olive oil, castor oil or arachis oil, a long or medium chain triglyceride, a mineral oil, such as liquid paraffin, or a mixture of these. Suitable emulsifying agents include naturally-occurring gums, such as gum acacia and gum tragacanth, naturally occurring phosphatides, such as soybean, phosphatidyl choline, glycerol, lecithin, esters or partial esters or salts derived from fatty acids and hexitol anhydrides, such as sorbitan monooleate, and condensation products of these partial esters with ethylene oxide, polyoxyethylene sorbitan monooleate, or a polyethoxylated castor oil such as Kolliphor EL, formerly known as Cremophor EL®. The emulsion may also contain sweetening and flavouring agents. Syrups and elixirs may be formulated with sweetening agents, such as glycerol, sorbitol or sucrose. Such compositions may also contain a demulcent, a preservative, flavouring or a colouring agent.

Pharmaceutical compositions of the second aspect of the invention may be in the form of a sterile injectable preparation, such as a sterile injectable aqueous or oleaginous suspension. This suspension may be formulated according to the known art using those suitable dispersing or wetting agents and suspending agents which have been mentioned herein. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

Pharmaceutical compositions of the second aspect of the invention suitable for administration to the eye include eye drops wherein the macrocyclic compound of the first aspect of the invention as active agent is dissolved or suspended in a suitable carrier, especially an aqueous solvent for the active ingredient. The active ingredient is preferably present in such formulations in a concentration of 0.5 to 20%, advantageously 0.5 to 10% particularly about 1.5% w/w.

Pharmaceutical compositions of the second aspect of the invention suitable for topical administration in the mouth include lozenges comprising the active agent in a flavored basis, usually sucrose and acacia or tragacanth; pastilles comprising the active agent in an inert basis such as gelatin and glycerin, or sucrose and acacia; and mouthwashes comprising the active agent in a suitable liquid carrier.

Pharmaceutical compositions of the second aspect of the invention for rectal administration may be presented as a suppository with a suitable base comprising for example cocoa butter or a salicylate.

Pharmaceutical compositions of the second aspect of the invention suitable for intrapulmonary or nasal administration have a particle size for example in the range of 0.1 to 500 μm (including particle sizes in a range between 0.1 and 500 μm in increments such as 0.5 μm, 1 μm, 30 μm, 35 μm, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of infections as described herein.

Pharmaceutical compositions of the second aspect of the invention suitable for vaginal administration may be presented as pessaries, tampons, creams, gels, pastes, foams or spray formulations containing in addition to the active agent such carriers as are known in the art to be appropriate.

Pharmaceutical compositions of the second aspect of the invention suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The pharmaceutical composition can be prepared to provide easily measurable amounts for administration. For example, an aqueous solution intended for intravenous infusion may contain from about 3 to 500 μg of the active agent per milliliter of solution in order that infusion of a suitable volume at a rate of about 30 mL/hr can occur.

The pharmaceutical compositions of the second aspect of the invention are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof of the macrocyclic compound of the first aspect of the invention as active agent.

Pharmaceutical compositions of the second aspect of the invention can also be formulated to provide controlled release of the active agent to allow less frequent dosing or to improve the pharmacokinetic or toxicity profile of the active agent. Accordingly, the invention also provides pharmaceutical compositions comprising one or more macrocyclic compounds of the first aspect of the invention formulated for sustained or controlled release.

The dosages may be varied depending upon the requirements of the patient, the severity of the condition being treated, and the macrocyclic compound of the first aspect of the invention being employed. Determination of the proper dosage for a particular situation is within the skill of the art. Generally, treatment is initiated with the smaller dosages which are less than the optimum dose of the macrocyclic compound. Thereafter the dosage is increased by small increments until the optimum effect under the circumstances is reached. For convenience, the total daily dosage may be divided and administered in portions during the day if desired.

The magnitude of an effective dose of a macrocyclic compound will, of course, vary with the nature of the severity of the condition to be treated and with the particular macrocyclic compound and its route of administration. The selection of appropriate dosages is within the ability of one of ordinary skill in this art, without undue burden. In general, the daily dose range may be from about 0.1 to about 100 mg per kg bodyweight of a human and non-human animal, preferably from about 1 to about 50 mg per kg of body weight of a human and non-human animal, and most preferably from about 3 to about 30 mg per kg of body weight of a human and non-human animal.

In a third aspect of the invention, a compound according to the first aspect of the invention is provided for use as a medicament.

The disclosed macrocyclic compounds of the first aspect of the invention may be used in medicine. A particular advantage of the macrocyclic compounds of the first aspect of the invention is their crossing the blood-brain barrier, which is an indispensable pre-requisite for the treatment of central nervous system (CNS) diseases. Therefore, the compounds of the first aspect of the invention can be used to prevent or treat diseases with a degenerative and inflammatory component of the pathophysiology, including stroke, Alzheimer's and Parkinson's disease, other synucleinopathies, epilepsy, motor neuron disease and amyotrophic lateral sclerosis (ALS), multiple sclerosis, Huntington's disease, ataxia, multiple system atrophy, spinal muscular atrophy, X-linked adenoleukodystrophy.

Further uses of the macrocyclic compounds of the first aspect of the invention include acute pancreatitis, sterile sepsis, multiple organ dysfunction, surgery-associated ischaemia/reperfusion injury, acute kidney injury associated with drug toxicity or rhabdomyolysis, and organ transplant conservation.

The macrocyclic compounds of the first aspect of the invention may be used in the manufacture of a medicament. In particular the macrocyclic compounds of the first aspect of the invention may be used in the manufacture of a medicament for the treatment of degenerative diseases of peripheral organs including acute pancreatitis; acute kidney injury caused by ischaemia and reperfusion, toxins, trauma, sepsis or rhabdomyolysis; haemodialysis-associated cardiac stunning; the CNS, including stroke, cognitive dysfunction associated with surgery or haemodialysis; Alzheimer's and Parkinson's disease; epilepsy; multiple sclerosis and amyotrophic lateral sclerosis (ALS); and bipolar disorder.

Thus, in a fourth aspect of the invention, a compound according to the first aspect of the invention is provided, for use in treating or preventing neurodegeneration; Alzheimer's disease; Parkinson's disease; X-linked adrenoleukodystrophy; epilepsy; amyotrophic lateral sclerosis (ALS); multiple sclerosis; bipolar disorder; stroke; hearing loss; diseases caused by cellular necrosis and inflammation; acute pancreatitis; surgery-associated acute kidney injury; acute kidney injury caused by toxic drugs; hearing loss caused by ototoxic drugs; acute kidney injury caused by aminoglycoside antibiotics; bacterial sepsis with an antibiotic; sepsis without an antibiotic; diseases wherein the subject has experienced or is suffering from physical trauma or crush injury, exposure to electrical current, extreme physical exertion or activity, and temperature extremes associated with or at risk for onset of rhabdomyolysis; diseases wherein the subject has a pre-existing condition or disease that increases the subject's risk of developing a kidney condition or disease when exposed to a nephrotoxin; diseases where a pre-existing condition or disease increases the risk of chronic kidney disease optionally wherein there is a history of renal impairment or a requirement for dialysis; diseases associated with reduced blood flow; cognitive dysfunction associated with surgery or haemodialysis; or cardiac stunning associated with haemodialysis.

In a fifth aspect of the invention, a compound according to the first aspect of the invention is provided, for use in protecting a transplant organ from ischaemia and inflammation.

### General Methods for Preparation of Macrocyclic Compounds of the Invention

The skilled person will recognise that macrocyclic compounds of Formula (I) may be prepared in a variety of ways, all including first the assembly of building blocks to give a linear precursor intermediate and ring closure typically as the final step.

For the compounds of the first aspect of the invention, macrocyclic ring closure has been achieved by three principal methods, involving the formation of (1) a carbon-oxygen bond, (2) of a carbon-nitrogen bond, and (3) of a carbon-carbon bond.

Carbon-oxygen bond formation is applied on linear precursors that have a carboxyl group on one end (the “C-terminus”) and a hydroxy group at the other end (the “O-terminus”). Carbon-nitrogen bond formation is applied on linear precursors that have a carboxyl group at the C-terminus and an amino group at the other end (the “N-terminus”). One route to carbon-carbon bond formation can be achieved by the so-called Heck coupling, which consists in the reaction of an unsaturated halide with an alkene in the presence of a base and a palladium catalyst.

In some cases, the building blocks required to assemble a linear precursor may be purchased. In other cases, such building blocks are known in the literature and can be synthesised using described procedures. In some cases, the linear precursor carries protective groups which may be removed after ring closure. The assembly of the linear precursor consists in joining the building blocks together using any of a large number of C—C, C—O, or C—N bond forming reactions available to skilled synthetic organic chemists.

The choice which bond to close in the final ring closing step depends on the individual case and the nature of the substituents in proximity to the bond to be formed by ring closure. The best yields are obtained if there is minimal steric bulk close to the reacting groups. Ring closure can be best achieved by forming either an ester bond (macro-lactonisation) or by forming an amide bond (macro-lactamisation). Alternatively, ring closure can be achieved by carbon-carbon bond formation using transition metal catalysts (for example Pd, Ni) or boronic acid reagents (for example Heck, Suzuki-Miyaura, Sonogashira, or Negichi reactions).

The general principle of macrocycle construction by assembling intermediates and final ring closure through macro-lactonisation is illustrated in Scheme 1 below for the preparation of Compound (2).

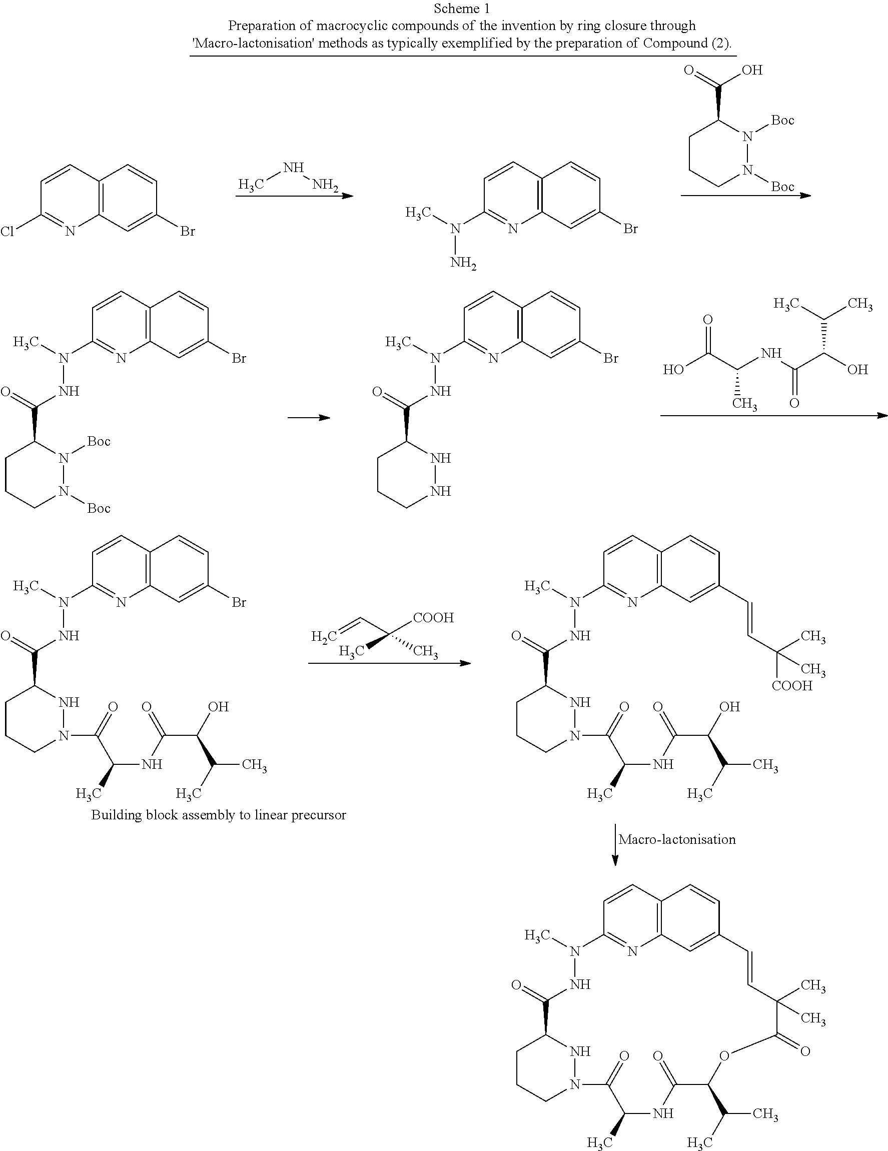

The general principle of macrocycle construction by assembling intermediates and final ring closure through macro-lactamisation is illustrated in Scheme 2 below for the preparation of Compound (16).

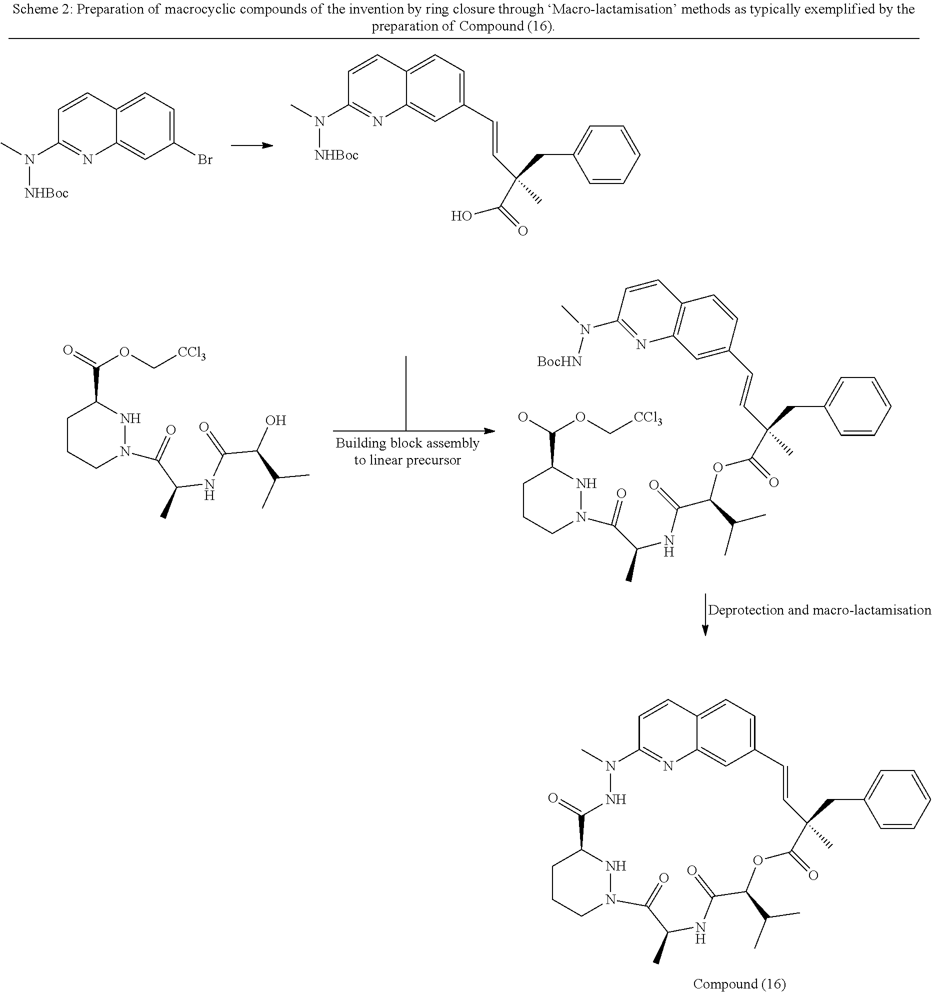

The general principle of macrocycle construction by assembling intermediate and final ring closure through intramolecular Heck coupling is illustrated in Scheme 3 below for the preparation of Compound (23).

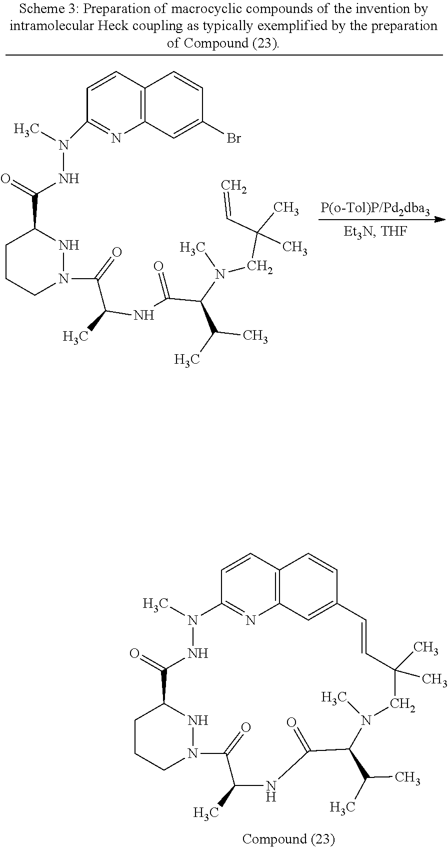 [SMILES: C=CC(C)(C)CN(C)[C@H](C(=O)N[C@@H](C)C(=O)N1CCC[C@@H](C(=O)NN(C)c2ccc3ccc(Br)cc3n2)N1)C(C)C ; CC(C)[C@H]1C(=O)N[C@@H](C)C(=O)N2CCC[C@H](N2)C(=O)NN(C)c2ccc3ccc(cc3n2)/C=C/C(C)(C)CN1C]

All three methods, macro-lactonisation, macro-lactamisation, and Heck coupling can independently be used to construct the macrocyclic compounds of the first aspect of the invention. The choice of the best method depends on the individual case. All compounds exemplified in Table 2 were prepared by modifications of Scheme 1, Scheme 2, or Scheme 3.

Cyclization to give the macrocyclic final products can be effected by treatment of a linear precursor with reagents that are commonly used to forming ester or amide bonds. Typically, such ring closing reactions are generally effected by conversion of the carboxylic acid group into an activated form which then reacts intramolecularly with the alcohol or amine group present at the other terminus of the linear precursor to form the macrocycle. Such reactions are often carried out under dilute conditions in an inert solvent such as, but not limited to, dichloromethane, toluene, acetonitrile, tert-butyl methylether and the like. Another method that may avoid the use of high dilution is to use flow reactors.

Preferred reagents for converting the carboxylic acid to an activated form include 2-methyl-6-nitrobenzoic anhydride, dicyclohexylcarbodiimide/N-hydroxysuccinimide; 1-ethyl-3-(3-dimethylaminopropyl)carbodiimide/N-hydroxybenzotriazole; 1-[bis(dimethylamino)methylene]-1H-1,2,3-triazolo[4,5-b]pyridinium-3-oxidhexafluorophosphate, and the like.

A core structure common to all macrocyclic compounds of the present invention consists of piperazic acid linked to a 2-hydrazino-N-heterocycle carrying carbon substituents at the 7-position, as shown below.

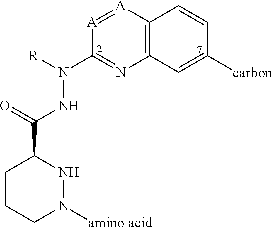

The addition of carbon substituents in position 7 of the N-heterocycle can conveniently be achieved by a palladium catalysed coupling reaction (Heck reaction) between a bromo-substituted heterocycle and a suitably substituted alkene. Typically the reaction is conducted by mixing the two reactants in an inert solvent along with a catalytic amount of a palladium reagent such as tris(dibenzylideneacetone)dipalladium(0) (Pd2dba3) and optionally heating the mixture to about 100° C. Preferably, the solvent is toluene, dichloroethane, tetrahydrofuran and the like and a wide variety of palladium reagents can be used including those derived from tri-ortho-tolyphosphine ligands.

Compounds similar to Compound (16) can be obtained by formation of an amide bond between the termini of a linear precursor having piperazic acid at its C-terminus and the 2-amino group of the N-terminal heterocyclic hydrazine.

N-heterocycles carrying hydrazines are known in the chemical literature and can be readily prepared by reacting 2-chloro-N-heterocycles with a substituted hydrazine such as, For example, methylhydrazine, benzylhydrazine (for Compound (9)) or (3-hydroxy)-propylhydrazine (for example in Compound (7)).

The invention will now be illustrated by the following examples.

### Example 1: Preparation of Compound (2)

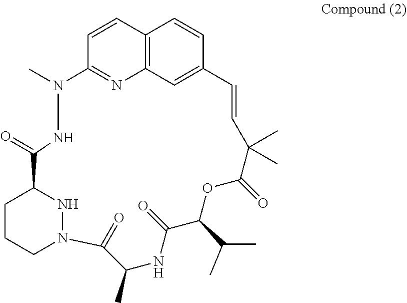 [SMILES: CC(C)[C@@H]1OC(=O)C(C)(C)/C=C/c2ccc3ccc(nc3c2)N(C)NC(=O)[C@@H]2CCCN(N2)C(=O)[C@H](C)NC1=O]

Step 1: Preparation of 1-(7-Bromo-2-quinolyl)-1-methylhydrazine (Intermediate (1)).

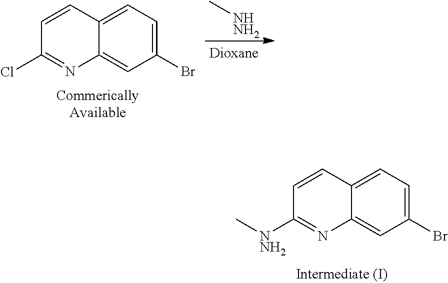

To a solution of 7-bromo-2-chloroquinoline (27.6 g, 114 mmol) in 1,4-dioxane (115 mL) was added methyl hydrazine (17.9 ml, 341 mmol). The stirred solution was heated at 90° C. for 2 hours. The reaction mixture was cooled and evaporated then dissolved in ethyl acetate. This was washed with water, saturated aqueous sodium bicarbonate and saturated brine, dried over magnesium sulphate, filtered and the solvent evaporated. This gave a yellow gum which was dried under vacuum to afford the title product, Intermediate (I), as a yellow solid (28.36 g) (LCMS M+H⁺=252, 254, C₁₀H₁₀BrN₃ requires 251, 253; RT=1.97 mins.; ¹H NMR (300 MHz, CDCl₃) δ 3.4 (2H, s) 4.25 (2H, s), 7.25-7.35 (2H, m), 7.45 (1H, d), 7.8 (1H, d), 7.9 (1H, d).

Step 2: Preparation of di-tert-butyl (3S)-3-[[(7-bromo-2-quinolyl)-methylamino]carbamoyl]hexahydropyridazine-1,2-dicarboxylate (Intermediate (II)).

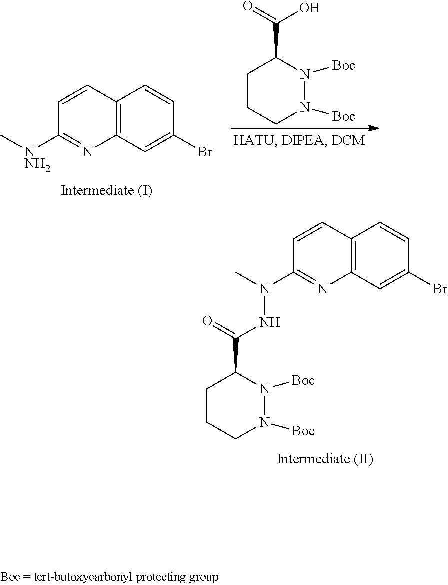

A solution of 1-(7-bromo-2-quinolyl)-1-methylhydrazine (20.0 g, 79.3 mmol) and (3S)-1,2-bis(tert-butoxycarbonyl)hexahydropyridazine-3-carboxylic acid (prepared according to the method described in Coats et al. (J. Org. Chem., 69, 1734-1737 (2004)) (26.2 g, 79.3 mmol) in dichloromethane (1 L) was cooled over an ice bath before adding N,N-diisopropylethylamine (DIPEA) (69.0 mL, 396.5 mmol) and [(dimethylamino)-1H-1,2,3-triazolo-[4,5-b]pyridin-1-ylmethylene]-N-methylmethanaminium hexafluorophosphate N-oxide (HATU) (36.2 g, 95.2 mmol). The ice bath was removed and the reaction was stirred for 16 hours at room temperature. The reaction mixture was washed with saturated sodium bicarbonate and saturated brine. This was dried over sodium sulphate, filtered and evaporated to give a yellow gum. The gum was purified by silica chromatography eluting with 30% ethyl acetate, 70% iso-hexane to afford an off-white foam. This was dried under vacuum to give the title product as a white solid (45.2 g) (LCMS M+H⁺=564, 566, C₂₅H₃₄BrN₅O₅ requires 563, 565; RT=3.65 mins.).

Step 3: Preparation of (25)-N-[(1S)-2-[(3S)-3-[[(7-Bromo-2-quinolyl)-methyl-amino]carbamoyl]hexahydropyridazin-1-yl]-1-methyl-2-oxo-ethyl]-2-hydroxy-3-methyl-butanamide (Intermediate (III)).

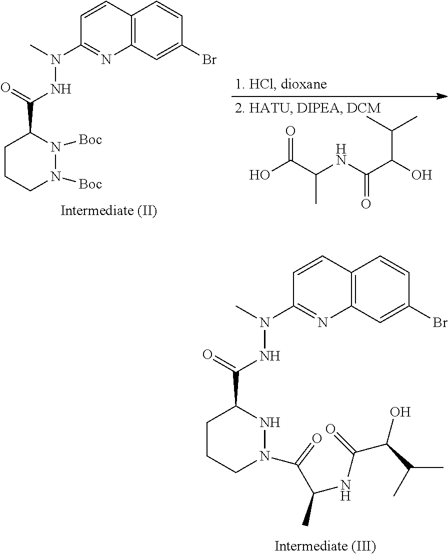 [SMILES: CN(NC(=O)[C@@H]1CCCN(C(=O)OC(C)(C)C)N1C(=O)OC(C)(C)C)c1ccc2ccc(Br)cc2n1 ; CC(NC(=O)C(O)C(C)C)C(=O)O ; CC(C)[C@H](O)C(=O)N[C@@H](C)C(=O)N1CCC[C@@H](C(=O)NN(C)c2ccc3ccc(Br)cc3n2)N1]

A solution of di-tert-butyl (3S)-3-[[(7-bromo-2-quinolyl)-methyl-amino]carbamoyl]hexahydro pyridazine-1,2-dicarboxylate (44.2 g, 78 mmol) in dichloromethane (600 mL) was cooled to 0° C. and 4 M HCl in dioxane (200 mL) was added. The reaction was allowed to warm to room temperature and was stirred for 16 hours. The mixture was evaporated to dryness then suspended in toluene and evaporated (×2). This afforded the intermediate amine hydrochloride as a white solid (39.93 g).

The material was suspended in dichloromethane (2.2 L) and (25)-2-[[(25)-2-hydroxy-3-methyl-butanoyl]amino]propanoic acid (14.75 g, 78 mmol) was added. The stirred mixture was cooled to 0° C. before adding N,N-diisopropylethylamine (69.4 mL, 390 mmol,) and [(dimethylamino)-1H-1,2,3-triazolo-[4,5-b]pyridin-1-ylmethylene]-N-methylmethanaminium hexafluorophosphate N-oxide (35.6 g, 93.6 mmol,). The ice bath was removed and the reaction was stirred at room temperature for 16 hours.

The reaction mixture was washed with saturated aqueous sodium bicarbonate and saturated aqueous ammonium chloride. This was dried over magnesium sulphate, filtered and evaporated to afford a yellow gum. The gum was purified by silica chromatography eluting with 2% methanol, 1% 880 ammonia, 97% ethyl acetate, then 4% methanol, 1% 880 ammonia, 95% ethyl acetate to give the title product as a yellow foam (26.18 g) (LCMS M+H⁺=535, 537, C₂₃H₃₁BrN₆O₄ requires 534, 536; RT=2.06 mins.).

Step 4: Preparation of (E)-4-[2-[[[(3S)-1-[(25)-2-[[(25)-2-Hydroxy-3-methyl-butanoyl]amino]propanoyl]hexahydropyridazine-3-carbonyl]amino]-methyl-amino]-7-quinolyl]-2,2-dimethyl-but-3-enoic acid (Intermediate (IV)).

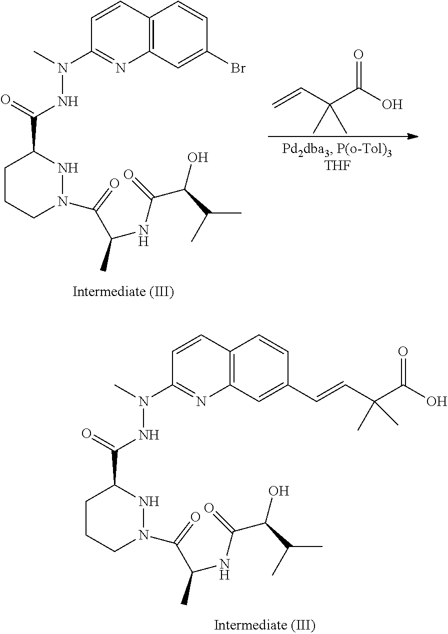

A solution of (25)-N-[(15)-2-[(3S)-3-[[(7-bromo-2-quinolyl)-methyl-amino]carbamoyl]hexahydropyridazin-1-yl]-1-methyl-2-oxo-ethyl]-2-hydroxy-3-methyl-butanamide (11.9 g, 20.9 mmol), 2,2-dimethylbutenoic acid (2.5 g, 22 mmol), tri(o-tolyl)phosphine (P(o-Tol)₃) (127 g, 4.18 mmol) and triethylamine (8.7 mL, 122 mmol) in tetrahydrofuran was purged with nitrogen for 20 minutes before adding tris(dibenzylideneacetone)dipalladium(0) (Pd₂dba₃) (1.91 g, 2.09 mmol). The stirred mixture was heated at reflux for 2 hours. The reaction was cooled, filtered through celite and evaporated to give an orange foam. The foam was dried under vacuum to yield the title product, as an orange solid (16.16 g) (LCMS M+H⁺=569, C₂₉H₄₀N₆O₆ requires 568; RT=1.60 mins.).

Step 5: Preparation of Compound (2) via ring closure through macro-lactonisation.

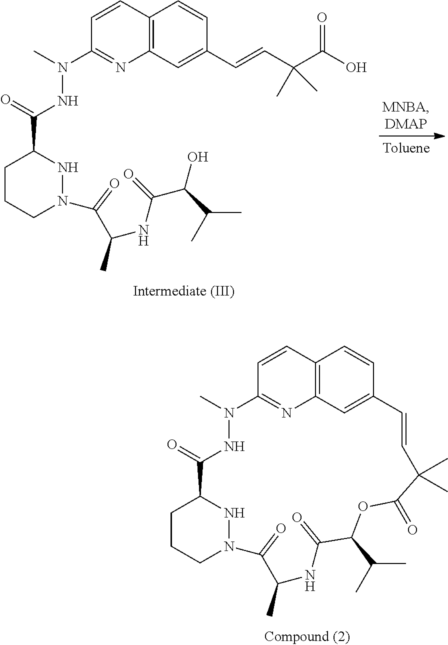 [SMILES: CC(C)[C@H](O)C(=O)N[C@@H](C)C(=O)N1CCC[C@@H](C(=O)NN(C)c2ccc3ccc(/C=C/C(C)(C)C(=O)O)cc3n2)N1 ; CC(C)[C@@H]1OC(=O)C(C)(C)/C=C/c2ccc3ccc(nc3c2)N(C)NC(=O)[C@@H]2CCCN(N2)C(=O)[C@H](C)NC1=O]

A 10 L flask fitted with mechanical stirring was charged with toluene (5 L) and powdered 4A molecular sieves (1 g) was added. The mixture was stirred for 20 minutes then left to stand for 16 hours under a nitrogen atmosphere. 2-Methyl-6-nitrobenzoic anhydride (MNBA) (10.32 g, 30 mmol) and 4-dimethylaminopyridine (DMAP) (7.32 g, 60 mmol) were added and the stirred mixture was heated to 100° C. A solution of (E)-4-[2-[[[(3S)-1-[(25)-2-[[(25)-2-hydroxy-3-methyl-butanoyl]amino]propanoyl]hexahydropyridazine-3-carbonyl]amino]-methyl-amino]-7-quinolyl]-2,2-dimethyl-but-3-enoic acid (11.6 g, 15 mmol) in anhydrous N,N-dimethylformamide (40 mL) was added dropwise over 6 hours using a syringe pump. The reaction mixture was stirred for a further 30 minutes at 100° C. before cooling to room temperature. The mixture was filtered and the filter cake was rinsed with further dichloromethane. The combined solution was evaporated to dryness. The material was then re-dissolved in dichloromethane before washing with saturated aqueous sodium bicarbonate and saturated aqueous ammonium chloride. The solution was filtered through a hydrophobic frit and evaporated to give an orange solid. The solid was purified by silica chromatography eluting with 0.5% 880 ammonia, 3% methanol and 96.5% ethyl acetate to yield a yellow solid (2.58 g).

This solid was combined with additional batches of product (9.52 g) prepared in a similar manner by dissolving in dichloromethane followed by evaporation to afford a yellow solid (12.10 g). The solid was broken up before adding diethyl ether (60 mL). The mixture was stirred for sixteen hours to give a fine pale-yellow suspension. The solid was collected by filtration and washed with diethyl ether, dried under vacuum to yield the title product (Compound (2)) as a pale yellow solid (10.16 g) (LCMS M+H⁺=551, C₂₉H₃₈N₆O₅ requires 550; RT=1.64 mins.; ¹H NMR (300 MHz, DMSO) δ 0.9 (6H, d), 1.1 (1H, t), 1.3 (3H, s), 1.4 (3H, s), 1.45 (3H, d), 1.6-1.9 (3H, m), 1.95-2.1 (1H, m), 2.7-2.85 (1H, m), 3.3-3.45 (1H, m), 3.35 (3H, s), 3.5 (1H, t), 4.25 (1H, d), 4.7 (1H, d), 5.1 (1H, d), 5.7-5.85 (1H, m), 6.2 (1H, d), 6.35 (1H, d), 7.15 (1H, d), 7.35 (1H, s), 7.7 (1H, d), 8.1 (1H, d), 8.6 (1H, d), 10.25 (1H, s)).

Compound (1) was prepared using the same procedure using but-3-enoic acid instead of 2,2-dimethyl-but-3-enoic acid as intermediate (¹H NMR (CDCl₃): 7.9 (m, 2H), 7.6 (m, 1H), 7.1 (m, 1H), 6.95 (d, 1H), 6.9 (m, 1H), 6.7 (m, 1H), 5.35 (m, 1H), 4.9 (m, 1H), 3.95 (m, 1H), 3.6-3.4 (m, 2H), 3.5 (s, 3H), 2.4 (m, 1H), 2.25 (m, 1H), 1.9 (m, 1H), 1.8-1.6 (m, 4H), 1.4 (m, 3H), 1.0 (m, 6H)).

Compound (3) is an isomer of Compound (2) (¹H NMR (CDCl₃): 8.05 (m, 1H), 7.7 (m, 2H), 7.3 (m, 1H), 7.1 (m, 1h), 6.6 (d, 1H), 6.35 (d, 1H), 5.75 (q, 1H), 4.95 (d, 1H), 4.5 (m, 1H), 3.6 (m, 1H), 3.4 (s, 3H), 2.85 (m, 1H), 2.3 (m, 1H), 2.0 (m, 2H), 1.7 (m, 2H), 1.6 (d, 3H), 1.55 (s, 3H), 1.4 (s, 3H), 0.95 (m, 6H)).

### Example 2: Preparation of Compound (5)

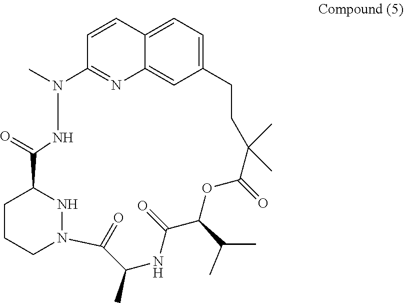 [SMILES: CC(C)[C@@H]1OC(=O)C(C)(C)CCc2ccc3ccc(nc3c2)N(C)NC(=O)[C@@H]2CCCN(N2)C(=O)[C@H](C)NC1=O]

Compound (2) from Example 1 (7 mg, 0.0127 mmol) was dissolved in ethanol (4 ml) and 10% Pd/C (3 mg) was added. H₂ was introduced and the reaction was stirred at room temperature for 1 h. The catalyst was filtered, washed with a small amount of ethanol and the solvent evaporated to afford the title Compound (5) as a colourless solid (6 mg) (LCMS M+H⁺=553, C₂₉H₄₀N₆O₅ requires 552; RT=1.71 mins.).

### Example 3: Preparation of Compound (6)

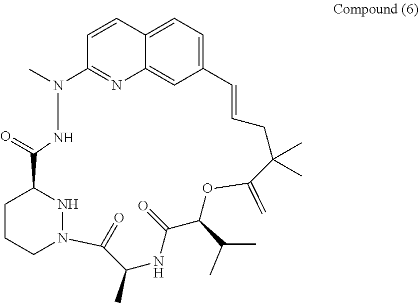 [SMILES: C=C1O[C@@H](C(C)C)C(=O)N[C@@H](C)C(=O)N2CCC[C@H](N2)C(=O)NN(C)c2ccc3ccc(cc3n2)/C=C/CC1(C)C]

Compound (6) was prepared in a manner similar to Example 1 by substituting 2,2-dimethylbut-3-enoic acid for 2,2-dimethylpent-4-enoic acid (Nicolai et al., Org. Lett., 12, 2, 384-7 (2010)) (LCMS M+H⁺=565, C₃₀H₄₀N₆O₅ requires 564; RT=1.59 mins.).

### Example 4: Preparation of Compound (4)

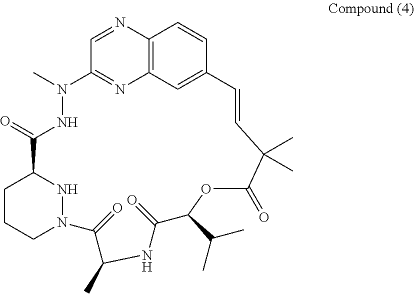 [SMILES: CC(C)[C@@H]1OC(=O)C(C)(C)/C=C/c2ccc3ncc(nc3c2)N(C)NC(=O)[C@@H]2CCCN(N2)C(=O)[C@H](C)NC1=O]

Compound (4) was synthesised in a similar manner to Example 1 above from 1-(7-Bromo-2-quinoxolyl)-1-methylhydrazine, itself synthesised from commercially available 7-bromo-2-chloro-quinoxaline analogous to step 1 of Example 1 (¹H NMR (CDCl₃): 8.6 (s, 1H), 7.8 (d, 1H), 7.6 (m, 1H), 7.5 (m, 1H), 6.3 (m, 2H), 5.9 (q, 1H), 5.1 (d, 1H), 4.4 (m, 1H), 3.6 (m, 1H), 3.5 (s, 3H), 2.8 (m, 1H), 2.2 (m, 1H), 1.9-1.7 (m, 4H), 1.55 (d, 3H), 1.5 (s, 3H), 1.4 (s, 3H), 1.0 (m, 6H); LCMS M+H⁺=552, C₂₈H₃₇NO₅ requires 551; RT=2.17 mins.).

### Example 5: Preparation of Compound (15)

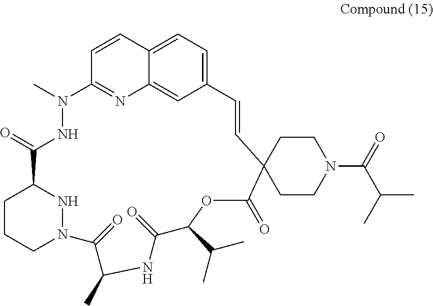 [SMILES: CC(C)C(=O)N1CCC2(/C=C/c3ccc4ccc(nc4c3)N(C)NC(=O)[C@@H]3CCCN(N3)C(=O)[C@H](C)NC(=O)[C@H](C(C)C)OC2=O)CC1]

Compound (15) was prepared by the macro-lactonisation route as described below.

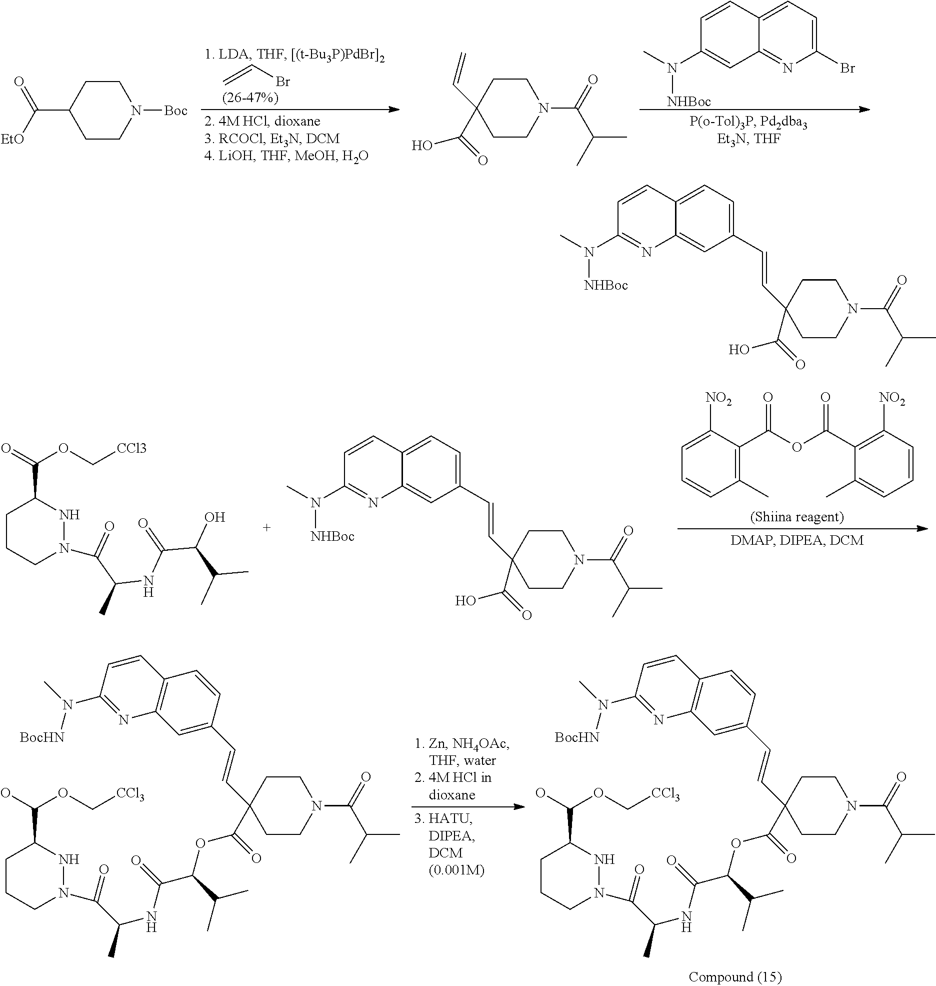

Step 1: Preparation of 1-(2-Methylpropanoyl)-4-vinyl-piperidine-4-carboxylic acid (Intermediate (V)).

Intermediate (V) was prepared in a four-step process as depicted below through Intermediates (Va), (Vb), and (Vc).

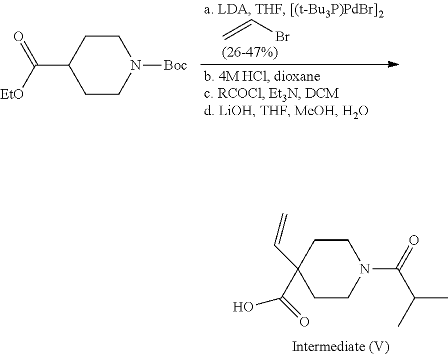

Step 1a: Preparation of 1-tert-Butyl 4-ethyl 4-vinylpiperidine-1,4-dicarboxylate (Intermedate (Va))

This step uses the method described by Bercot et al. (Organic Letters, 10, 5251-5254 (2008)). LDA is lithium diisopropyl amide.

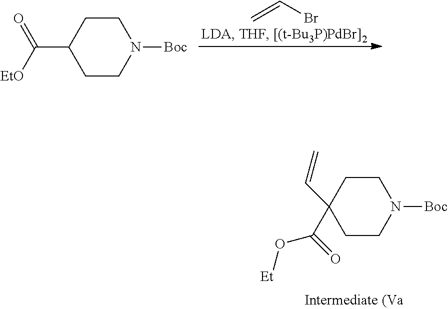

Step 1b: Preparation of Ethyl 4-vinylpiperidine-4-carboxylate (Intermediate (Vb)).

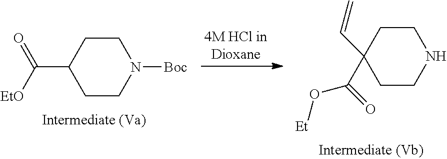 [SMILES: CCOC(=O)C1CCN(C(=O)OC(C)(C)C)CC1 ; C=CC1(C(=O)OCC)CCNCC1]

A solution of 1-tert-butyl 4-ethyl 4-vinylpiperidine-1,4-dicarboxylate (1.3 g, 4.5 mmol) in 1,4-dioxane (20 mL) was stirred at room temperature. 4 M Hydrogen chloride in 1,4-dioxane (10 mL) was added dropwise over 1 min and the reaction mixture stirred for 2 h. The solvent was evaporated and the residue was co-evaporated with toluene (×2) to afford the title compound (4.5 mmol) (LCMS (m/z) 184 [M+H], RT=0.25 min.).

Step 1c: Preparation of Ethyl 1-(2-methylpropanoyl)-4-vinyl-piperidine-4-carboxylate (Intermediate (Vc)).

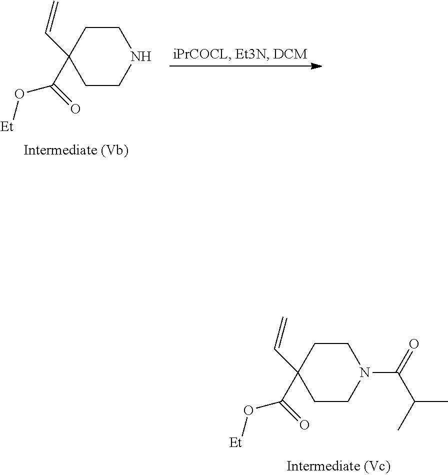 [SMILES: C=CC1(C(=O)OCC)CCNCC1 ; C=CC1(C(=O)OCC)CCN(C(=O)C(C)C)CC1]

A solution of ethyl 4-vinylpiperidine-4-carboxylate (4.5 mmol) in dichloromethane (20 mL) was stirred at 0° C. Triethylamine (1.42 g, 2 mL, 14 mmol) was added followed by a solution of 2-methylpropanoyl chloride (750 mg, 0.75 mL, 7 mmol) in dichloromethane (10 mL). The reaction mixture was stirred at room temperature for 18 hours. Water was added and the mixture was washed with 2 M hydrochloric acid. The organic layer was separated, washed with water, saturated sodium hydrogen carbonate solution, water and brine. The organic layer was separated, filtered through a hydrophobic frit and the filtrate was evaporated. The residue was purified by silica gel chromatography eluting with 0-50% ethyl acetate in iso-hexane to afford the title compound (944 mg, 79%) as an oil (LCMS (m/z) [M+H]254, RT=2.21 min.).

Step 1d: Preparation of 1-(2-Methylpropanoyl)-4-vinyl-piperidine-4-carboxylic acid (Intermediate (V)).

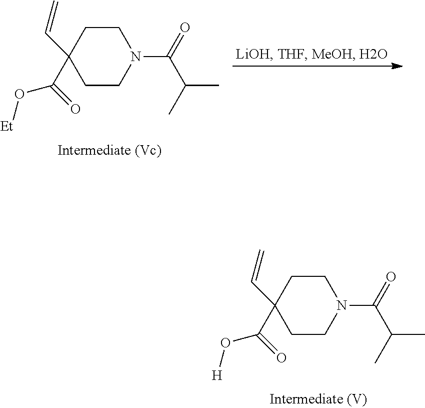 [SMILES: C=CC1(C(=O)OCC)CCN(C(=O)C(C)C)CC1 ; C=CC1(C(=O)O)CCN(C(=O)C(C)C)CC1]

A solution of ethyl 1-(2-methylpropanoyl)-4-vinyl-piperidine-4-carboxylate (944 mg, 3.7 mmol) in tetrahydrofuran (30 mL) was stirred at 5° C. under nitrogen. A solution of lithium hydroxide hydrate (466 mg, 11.1 mmol) in water (10 mL) and methanol (3 mL) was added and the reaction mixture was stirred at room temperature for 21 hours. The majority of the organic solvent was evaporated, the mixture was acidified to pH 1-2 with 2 M hydrochloric acid and the resulting suspension was extracted with ethyl acetate. The organic extracts were combined, washed with brine. The organic layer was filtered through a hydrophobic frit and the filtrate was evaporated to afford the title compound (815 mg, 97%) as an oil (LCMS (m/z) 226 [M+H], RT=1.47 min.).

Step 2: Preparation of 4-[(E)-2-[2-[(tert-Butoxycarbonylamino)-methyl-amino]-7-quinolyl]vinyl]-1-(2-methylpropanoyl)piperidine-4-carboxylic acid (Heck Reaction) (Intermediate (VI)).

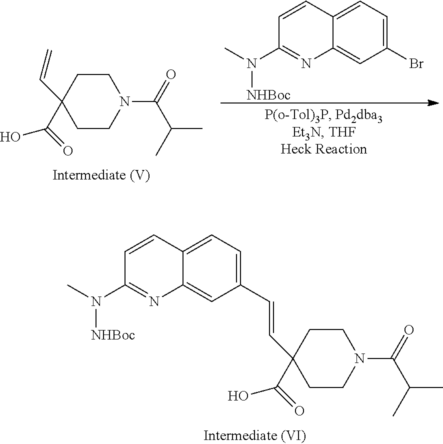 [SMILES: C=CC1(C(=O)O)CCN(C(=O)C(C)C)CC1 ; CN(NC(=O)OC(C)(C)C)c1ccc2ccc(Br)cc2n1 ; CC(C)C(=O)N1CCC(/C=C/c2ccc3ccc(N(C)NC(=O)OC(C)(C)C)nc3c2)(C(=O)O)CC1]

To a stirred solution of tert-butyl N-[(7-bromo-2-quinolyl)-methyl-amino]carbamate, 4-oxo-1-vinylcyclohexanecarboxylic acid (352 mg, 1 mmol) and 1-(2-methylpropanoyl)-4-vinyl-piperidine-4-carboxylic acid (225 mg, 1 mmol) in tetrahydrofuran (30 mL) was added tri-(o-tolyl)phosphine (61 mg, 0.0.2 mmol) and triethylamine (303 mg, 0.42 mL, 3 mmol). The reaction mixture was degassed with nitrogen for 10 minutes. Bis(dibenzylideneacetone)palladium(0) (92 mg, 0.1 mmol) was added, the reaction mixture was heated at reflux under nitrogen for 1 hour. The reaction was cooled to room temperature and the mixture was filtered through a frit and the filtrate was evaporated. The residue was co-evaporated with toluene (×3) to give the title compound (1 mmol), an orange oil, which was used crude in the next step (LCMS (m/z) 497 [M+H], RT=1.56 min.).

Step 3: Preparation of 2,2,2-Trichloroethyl(3S)-1-[(2S)-2-[[(2S)-2-[4-[(E)-2-[2-[(tert-butoxycarbonylamino)-methylamino]-7-quinolyl]vinyl]-1-(2-methylpropanoyl)piperidine-4-carbonyl]oxy-3-methylbutanoyl]amino]propanoyl]hexahydropyridazine-3-carboxylate (Intermediate (VII)).

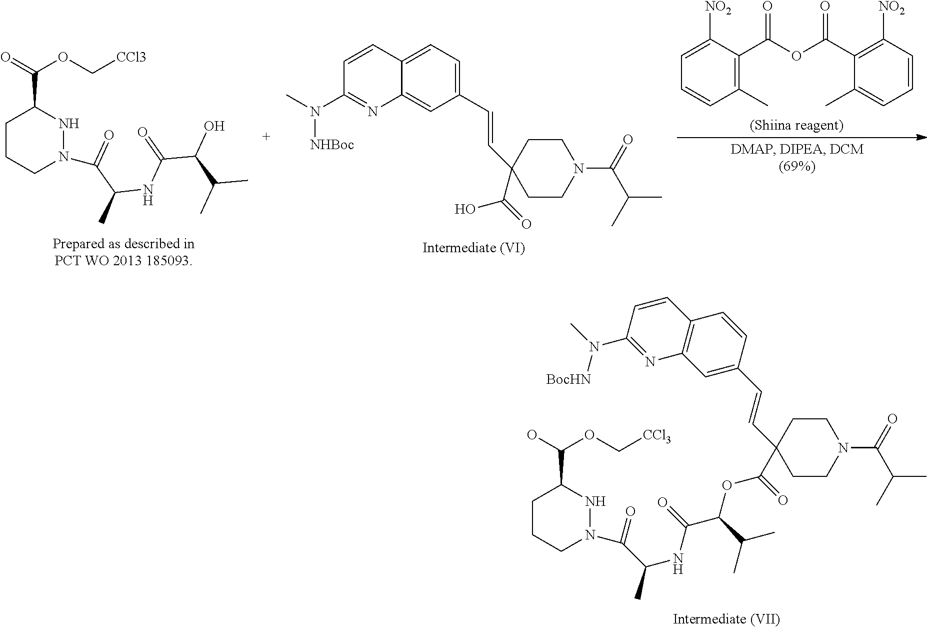

The title compound was prepared from 4-[(E)-2-[2-[(tert-butoxycarbonylamino)-methyl-amino]-7-quinolyl]vinyl]-1-(2-methylpropanoyl)piperidine-4-carboxylic acid and 2,2,2-trichloroethyl(3S)-1-[(2S)-2-[[(2S)-2-hydroxy-3-methyl-butanoyl]amino]propanoyl]hexahydropyridazine-3-carboxylate, prepared as described in WO 2013/185093 (Gilead Sciences Inc. and Selcia Limited) in 69% yield using the method described in Example 1 (LCMS (m/z) 910/912/914 [M+H], RT=2.51 min.).

Step 4: Preparation of Compound (15) via ring closure by macro-lactamisation.

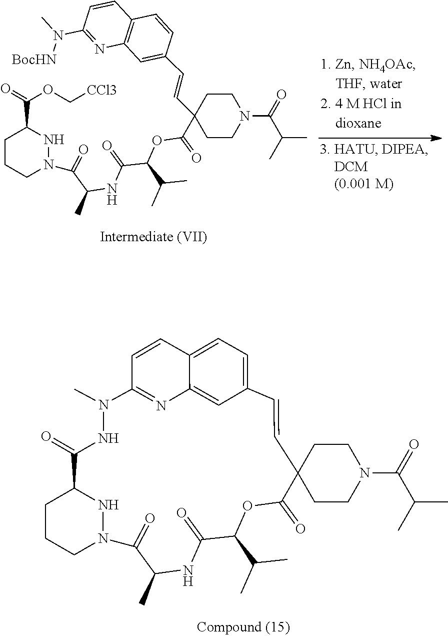

The title Compound (15) was prepared from 2,2,2-trichloroethyl(3S)-1-[(2S)-2-[[(2S)-2-[4-[(E)-2-[2-[(tertbutoxycarbonylamino)-methyl-amino]-7-quinolyl]vinyl]-1-(2-methylpropanoyl)piperidine-4-carbonyl]oxy3-methyl-butanoyl]amino]propanoyl]hexahydropyridazine-3-carboxylate in 15% yield (over 3 steps) using the method described in Example 1 (LCMS (m/z) 662 [M+H], RT=1.57 min.; ¹H NMR (CDCl₃): δ 8.05 (d, 1H), 7.6 (d, 1H), 7.45 (m, 2H), 7.1 (d, 1h), 6.4 (d, 1H), 6.35 (d, 1H), 6.0 (m, 1H), 5.2 (m, 1H), 4.4 (m, 2H), 4.0 (m, 1H), 3.6 (m, 1H), 3.4 (s, 3H), 3.0 (m, 1H), 2.8 (m, 2H), 2.5 (m, 1H), 2.2 (m, 2H), 1.9-1.6 (m, 7H), 1.55 (d, 3H), 1.1 (m, 6H), 1.0 (m, 6H)).

### Example 6: Preparation of Compound 17

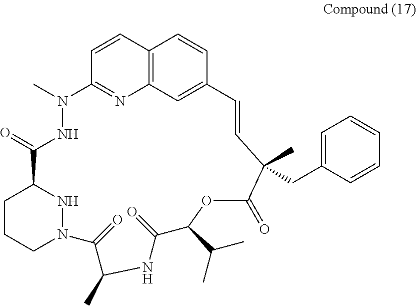 [SMILES: CC(C)[C@@H]1OC(=O)[C@@](C)(Cc2ccccc2)/C=C/c2ccc3ccc(nc3c2)N(C)NC(=O)[C@@H]2CCCN(N2)C(=O)[C@H](C)NC1=O]

Compound (17) was prepared in a manner similar to that to Compound (15) in Example 5 above but using (2R)-2-benzyl-2-methyl-but-3-enoic acid instead of ethyl 1-(2-methylpropanoyl)-4-vinyl-piperidine-4-carboxylate as described below.

Preparation of (2R)-2-benzyl-2-methyl-but-3-enoic acid Intermediate (VIII).

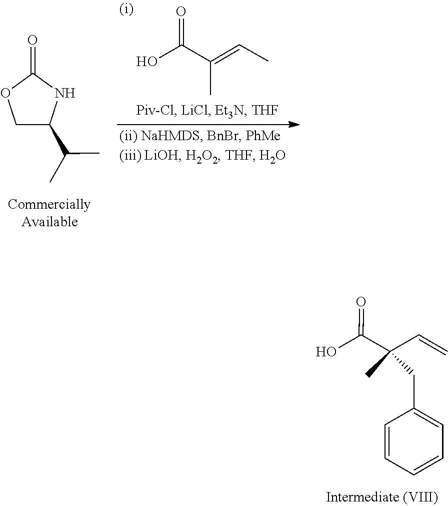

Intermediate (VIII) was prepared in a 3 step process as follows.

Step 1: Preparation of (4S)-4-isopropyl-3-[(E)-2-methylbut-2-enoyl]oxazolidin-2-one.

Pivaloyl chloride (Piv-Cl) (6.23 mL, 50.7 mmol) was added dropwise to a solution of the tiglic acid (4.6 g, 46.07 mmol) and trimethylamine (14.13 mL, 101.35 mmol) in THF (80 mL) at −20° C. under N₂ and the reaction was stirred for 30 minsutes. Lithium chloride (2.34 g, 55.28 mmol) was added in one portion followed by the oxazolidinone (5.95 g, 46.07 mmol) in THF (15 mL) and the reaction was stirred with slow warming to room temperature overnight. The white suspension was quenched with saturated aqueous NH₄Cl and stirred for 5 minsutes. The solvent was evaporated and the residue was portioned between water and ethyl acetate. The organic layer was further washed with saturated aqueous NaHCO₃, dried (MgSO₄) and the solvent evaporated to afford a crude oil. This was purified by column chromatography (120 g, 0 to 50% EtOAc in hexanes) to afford the title compound as a white solid (8.19 g) (¹H NMR (300 MHz, MeOD) δ 0.85 (dd, 6H), 1.80 (d, 3H), 1.90 (s, 3H), 2.35 (m, 1H), 4.15 (m, 1H), 4.30 (t, 1H), 4.50 (m, 1H), 6.20 (m, 1H)).

Step 2: Preparation of (4S)-3-[(2R)-2-benzyl-2-methyl-but-3-enoyl]-4-isopropyl-oxazolidin-2-one.

Sodium hexamethldisilazide (NaHMDS) (20.5 mL, 12.32 mmol, 0.6 M in toluene (PhMe)) was added dropwise to a solution of the (4S)-4-isopropyl-3-[(E)-2-methylbut-2-enoyl]oxazolidin-2-one (2 g, 9.48 mmol) in toluene (50 mL) at −78° C. and the reaction was stirred for 40 minutes. Benzyl bromide (BnBr) (1.69 mL, 14.22 mmol) was added dropwise and the reaction was allowed to stir for 3 hours with slow warming to 0° C. The reaction was quenched by the addition of saturated aqueous NH₄Cl and the reaction was stirred for 5 minutes. The phases were separated and the organic layer was dried (MgSO₄) and the solvent evaporated to afford a crude oil. This was purified by column chromatography (80 g, 0 to 20% EtOAc in hexanes) to afford the title compound as a colourless oil (493 mg). Minor isomer (192 mg) and recovered starting material (801 mg) were also isolated (¹H NMR (300 MHz, CDCl3) δ 0.85 (m, 6H), 1.45 (s, 3H), 2.30 (m, 1H), 3.35 (dt, 2H), 4.20 (m, 2H), 4.45 (m, 1H), 4.95 (m, 1H), 5.10 (m, 1H), 6.30 (m, 1H), 7.25 (m, 5H)).

Step 3: Preparation of (2R)-2-benzyl-2-methyl-but-3-enoic acid (Intermediate (VII)).

Hydrogen peroxide (966 μL, 8.53 mmol, 30% wt in H2O) was added to a mixture of the (4S)-3-[(2R)-2-benzyl-2-methyl-but-3-enoyl]-4-isopropyl-oxazolidin-2-one (493 mg, 1.71 mmol) and lithium hydroxide hydrate (143 mg, 3.41 mmol) in THF (8 mL) and water (4 mL) and the reaction was stirred for 5 hours. The reaction was quenched with sodium metabisulfite, stirred for 5 minutes, before adding 2 M NaOH (pH=14). The mixture was extracted with diethyl ether (2×). The aqueous layer was acidified with 1 M HCl (pH˜2) then extracted with diethyl ether (2×). The organic layer was dried (MgSO₄) and the solvent evaporated to afford the title compound as an oil (185 mg) (¹H NMR (300 MHz, CDCl3) δ 1.25 (s, 3H), 2.90 (d, 1H), 3.10 (d, 1H), 5.15 (m, 2H), 6.10 (dd, 1H), 7.25 (m, 51H)).

### Final Steps: Preparation of Compound (17).

(2R)-2-benzyl-2-methyl-but-3-enoic acid (Intermediate (VII) was then further elaborated following the method described for Example 5 to provide Compound (17) (LCMS (m/z) 627 [M+H], RT=2.01 min.; C₃₅H₄₂N₆O₅ requires 626).

### Example 7: Preparation of Compound (81)

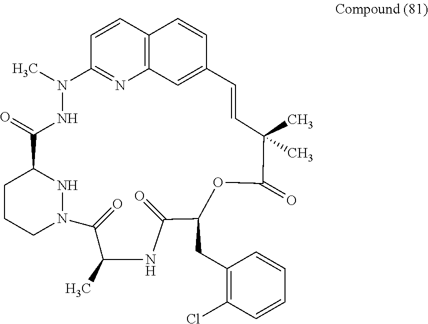

Compound (81) was prepared in a multistep process starting from from di-tert-butyl (3S)-3-[[(7-bromo-2-quinolyl)-methylamino]carbamoyl]hexahydropyridazine-1,2-dicarboxylate (synthesis described in Example 1 as Intermediate (II)).

Step 1: Preparation of (3S)-N′-(7-bromo-2-quinolyl)-N′-methyl-hexahydropyridazine-3-carbohydrazide dihydrochloride (Intermediate (IX)).

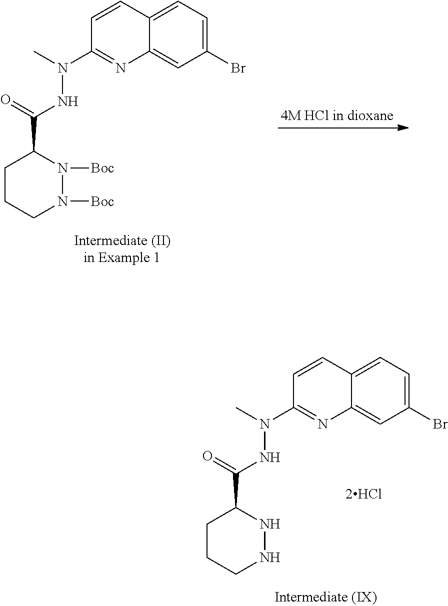 [SMILES: CN(NC(=O)[C@@H]1CCCN(C(=O)OC(C)(C)C)N1C(=O)OC(C)(C)C)c1ccc2ccc(Br)cc2n1 ; CN(NC(=O)[C@@H]1CCCNN1)c1ccc2ccc(Br)cc2n1]

To a solution of di-tert-butyl (3S)-3-[[(7-bromo-2-quinolyl)-methylamino]carbamoyl]hexahydropyridazine-1,2-dicarboxylate (22.3 g, 39.7 mmol) in dichloromethane (400 mL) at 0° C. was added 4 M hydrochloric acid in dioxane (100 mL). The reaction mixture was allowed to warm to room temperature and then stirred for 16 hours. The reaction was concentrated at reduced pressure and then suspended in toluene and concentrated (×2) to afford the title compound as a yellow solid (17.21 g, ˜100%) (¹H NMR (300 MHz, CD₃OD) δ 1.94-2.10 (m, 3H), 2.22 (m, 1H), 3.22 (m, 1H), 3.34 (m, 1H), 3.64 (s, 3H), 4.11 (m, 1H), 7.49 (d, J=9.6 Hz, 1H), 7.77 (dd, J=8.6, 1.7 Hz, 1H), 7.92 (d, J=8.6 Hz, 1H), 8.50 (br s, 1H), 8.65 (d, J=9.6 Hz, 1H); LCMS (m/z) 252, 254 [M+H]).

Step 2: Preparation of tert-butyl N-[(1S)-2-[(3S)-3-[[(7-bromo-2-quinolyl)-methylamino]carbamoyl]hexahydropyridazin-1-yl]-1-methyl-2-oxo-ethyl]carbamate (Intermediate (X)).

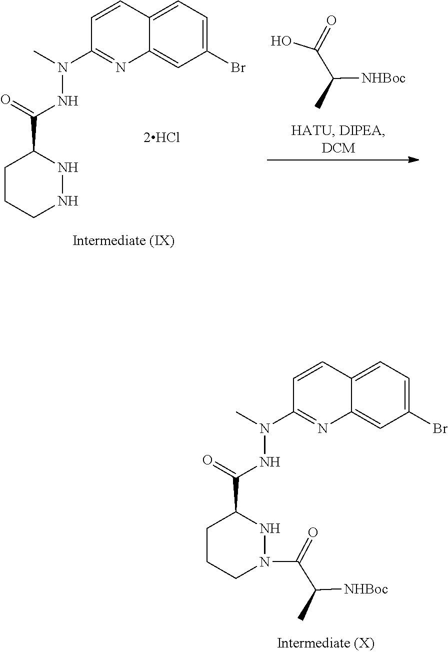 [SMILES: CN(NC(=O)[C@@H]1CCCNN1)c1ccc2ccc(Br)cc2n1 ; C[C@H](NC(=O)OC(C)(C)C)C(=O)O ; C[C@H](NC(=O)OC(C)(C)C)C(=O)N1CCC[C@@H](C(=O)NN(C)c2ccc3ccc(Br)cc3n2)N1]

To a solution of (3S)-N′-(7-bromo-2-quinolyl)-N′-methyl-hexahydropyridazine-3-carbohydrazide dihydrochloride (9.0 g, 20.7 mmol) in dichloromethane (400 mL) at 0° C. was added Boc-L-alanine (4.0 g, 21.1 mmol), N,N-diisopropylethylamine (18 mL, 103.5 mmol) followed by 0-(7-azabenzotriazol-1-yl)-N,N,N′,N′-tetramethyluronium hexafluorophosphate (11.1 g, 29.2 mmol). The reaction was allowed to warm to room temperature and then stirred for a further 18 hours. The reaction mixture was diluted with dichloromethane and washed with water, saturated aqueous sodium hydrogen carbonate solution and then brine, dried over magnesium sulphate, filtered and concentrated. The product was purified by silica gel chromatography eluting with 0-100% acetone in isohexane to afford the title compound as a pale yellow solid (11.29 g, ˜100%) (¹H NMR (300 MHz, CD₃OD) δ 1.27-1.35 (m, 12H), 1.67-1.95 (m, 3H), 2.07 (m, 1H), 3.14 (m, 1H), 3.45 (s, 3H), 3.64 (m, 1H), 4.10 (m, 1H), 5.16 (m, 1H), 7.06 (d, J=9.0 Hz, 1H), 7.37 (dd, J=8.6, 1.8 Hz, 1H), 7.59 (d, J=8.6 Hz, 1H), 7.89 (d, J=1.7 Hz), 8.00 (d, J=8.7 Hz, 1H); LCMS (m/z) 535, 537 [M+H]).

Step 3: Preparation of (3S)-1-[(2S)-2-aminopropanoyl]-N′-(7-bromo-2-quinolyl)-N′-methyl-hexahydropyridazine-3-carbohydrazide hydrochloride (Intermediate (XI)).

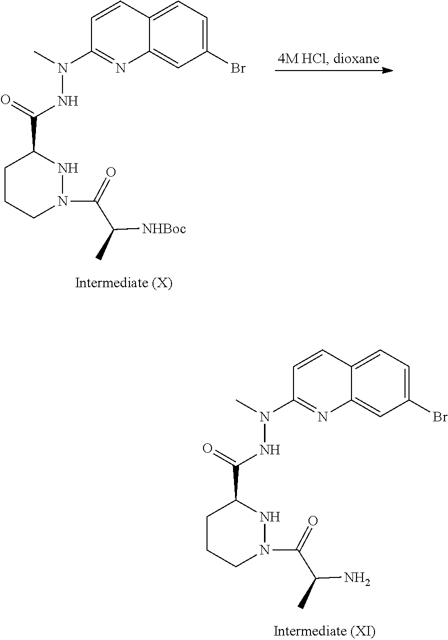 [SMILES: C[C@H](NC(=O)OC(C)(C)C)C(=O)N1CCC[C@@H](C(=O)NN(C)c2ccc3ccc(Br)cc3n2)N1 ; C[C@H](N)C(=O)N1CCC[C@@H](C(=O)NN(C)c2ccc3ccc(Br)cc3n2)N1]

To a solution of tert-butyl N-[(1S)-2-[(3S)-3-[[(7-bromo-2-quinolyl)-methylamino]carbamoyl]hexahydropyridazin-1-yl]-1-methyl-2-oxo-ethyl]carbamate (11.2 g, 21.1 mol) in dichloromethane (400 mL) at 0° C. was added 4 M hydrogen chloride in dioxane (30 mL, 120 mmol). The reaction mixture was allowed to warm to room temperature and stirred for 18 hours. The reaction mixture was concentrated in vacuo and then the resulting solid triturated with diethyl ether to afford the title compound (slightly contaminated with tetramethylurea) as a pale yellow solid (10.2 g, >100%) (¹H NMR (300 MHz, CD3OD) δ 1.53 (d, J=6.7 Hz, 3H) 1.77 (m, 1H), 1.92 (m, 2H), 2.21 (m, 1H), 3.64 (m, 1H), 3.66 (s, 3H), 3.86 (m, 1H), 4.01 (m, 1H), 4.93 (m 1H), 7.48 (d, J=9.6 Hz, 1H), 7.77 (dd, J=8.7, 1.8 Hz, 1H), 7.92 (d, J=8.8 Hz, 1H), 8.46 (br s, 1H), 8.57 (d, J=10.0 Hz, 1H); LCMS (m/z) 435, 437 [M+H]).

Step 4: Preparation of (2S)-N-[(1S)-2-[(3S)-3-[[(7-bromo-2-quinolyl)-methyl-amino]carbamoyl]hexahydropyridazin-1-yl]-1-methyl-2-oxo-ethyl]-3-(2-chlorophenyl)-2-hydroxy-propanamide. Intermediate (XII).

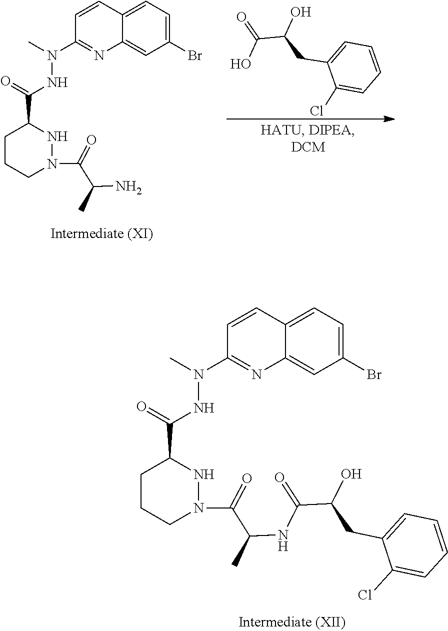 [SMILES: C[C@H](N)C(=O)N1CCC[C@@H](C(=O)NN(C)c2ccc3ccc(Br)cc3n2)N1 ; O=C(O)[C@@H](O)Cc1ccccc1Cl ; C[C@H](NC(=O)[C@@H](O)Cc1ccccc1Cl)C(=O)N1CCC[C@@H](C(=O)NN(C)c2ccc3ccc(Br)cc3n2)N1]

To a solution of (3S)-1-[(2S)-2-aminopropanoyl]-N′-(7-bromo-2-quinolyl)-N′-methylhexahydropyridazine-3-carbohydrazide hydrochloride (479 mg, 1.02 mmol) in dichloromethane (40 mL) at 0° C. was added (2S)-3-(2-chlorophenyl)-2-hydroxy-propanoic acid (210 mg, 1.05 mmol), N,N-diisopropylethylamine (1 mL, 5.75 mmol) followed by 0-(7-azabenzotriazol-1-yl)-N,N,N′,N′-tetramethyluronium hexafluorophosphate (580 mg, 1.52 mmol). The reaction was allowed to warm to room temperature and then stirred for a further 18 hours. The reaction mixture was diluted with dichloromethane and washed with water, saturated aqueous sodium hydrogen carbonate solution and then brine, dried over magnesium sulphate, filtered and concentrated. The product was purified by silica gel chromatography eluting with 0-100% acetone in isohexane to afford the title compound as a pale yellow solid (382 mg, 95%) (¹H NMR (300 MHz, CD₃OD) δ 1.36 (d, J=6.9 Hz, 3H), 1.63-2.00 (m, 3H), 2.09 (m, 1H), 2.84 (dd, J=14.0, 9.0 Hz, 1H), 3.1 (m, 1H), 3.44 (s, 3H), 3.65 (m, 1H), 4.14 (br. d, J=12.7 Hz, 1H), 4.25 (dd, J=8.9, 3.8 Hz, 1H), 5.17 (d, J=10.5 Hz, 1H), 5.41 (q, J=6.9 Hz, 1H), 7.06 (d, J=9.1 Hz, 1H), 7.15-7.22 (m, 2H), 7.27-7.34 (m, 2H), 7.38 (dd, J=8.7, 1.8 Hz, 1H), 7.60 (d, J=8.7 Hz, 1H), 7.89 (d, J=1.8 Hz. 1H), 8.01 (d, J=8.7 Hz, 1H); LCMS (m/z) 619, 621 [M+H]).

Step 5: Preparation of (E)-4-[2-[[[(3S)-1-[(2S)-2-[[(2S)-3-(2-chlorophenyl)-2-hydroxypropanoyl]amino]propanoyl]hexahydropyridazine-3-carbonyl]amino]-methyl-amino]-7-quinolyl]-2,2-dimethyl-but-3-enoic acid (Intermediate (XIII)).

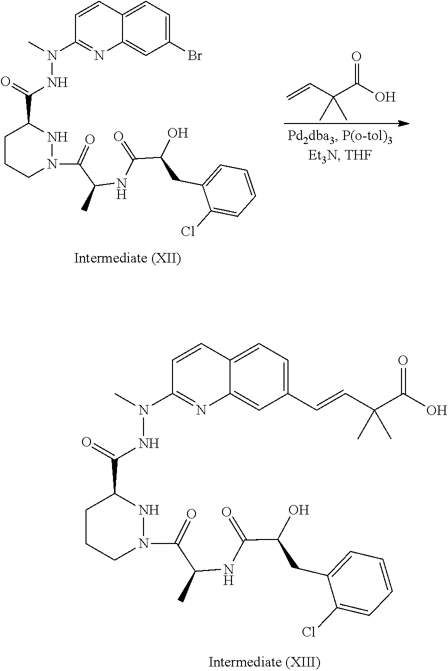

To a solution of (2S)-N-[(1S)-2-[(3S)-3-[[(7-bromo-2-quinolyl)methylamino]carbamoyl]hexahydropyridazin-1-yl]-1-methyl-2-oxo-ethyl]-3-(2-chlorophenyl)-2-hydroxypropanamide (380 mg, 0.62 mmol) in tetrahydrofuran (25 mL) was added 2,2-dimethyl-3-butenoic acid (73 mg, 0.64 mmol), tri(o-tolyl)phosphine (35 mg, 0.12 mmol) and triethylamine (0.26 mL, 1.87 mmol). The reaction mixture was purged with nitrogen for 5 minutes and then treated with tris(dibenzylideneacetone)dipalladium(0) (57 mg, 0.062 mmol). The reaction was heated to reflux for 1 hour and then allowed to cool to room temperature. The mixture was filtered through a pad of celite which was washed with ethyl acetate. The filtrate was concentrated at reduced pressure and the resulting residue triturated with diethyl ether. The solid was collected by filtration, washed with diethyl ether and dried in vacuo to yield the crude title compound as a yellow solid (328 mg, 81%) (LCMS (m/z) 651, 653 [M+H]).

### Step 6. Preparation of Compound (81).

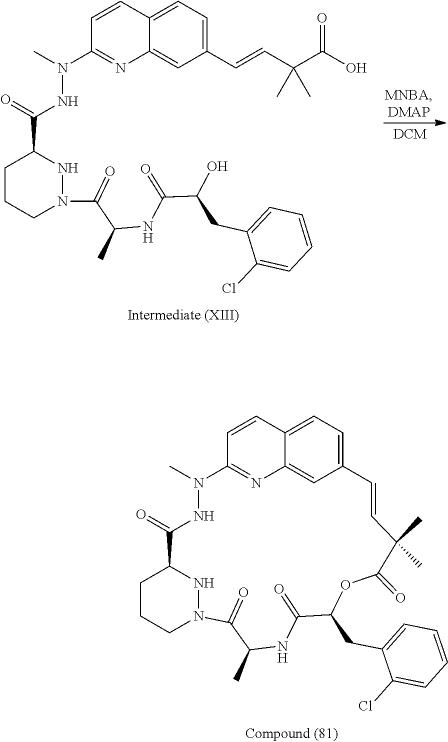

A solution of (E)-4-[2-[[[(3S)-1-[(2S)-2-[[(2S)-3-(2-chlorophenyl)-2-hydroxypropanoyl]amino]propanoyl]hexahydropyridazine-3-carbonyl]amino]-methyl-amino]-7-quinolyl]-2,2-dimethyl-but-3-enoic acid (280 mg, 0.43 mmol) in dichloromethane (20 mL) and N,Ndimethylformamide (1 mL) was added via syringe pump to a solution of 2-methyl-6-nitrobenzoic anhydride (340 mg, 0.99 mmol) and 4-dimethylaminopyridine (262 mg, 2.15 mmol) in dichloromethane (200 mL) containing powdered 4A molecular sieves at reflux over 4 hours. The reaction mixture was heated at reflux for a further 2 hours and then stirred at room temperature for 16 hours. The mixture was filtered through a pad of celite and then the solution was washed successively with saturated aqueous ammonium chloride, saturated aqueous sodium hydrogen carbonate and brine. The organic solution was passed through a hydrophobic frit and then the solution was concentrated at reduced pressure. The product was purified by reverse phase preparative HPLC eluting with acetonitrile/water containing 0.1% formic acid. Relevant fractions were combined and basified with solid sodium hydrogen carbonate, then partially concentrated to remove the acetonitrile. The product was extracted into ethyl acetate (×2), dried over magnesium sulphate, filtered and concentrated to afford compound (81) as a yellow solid (52 mg, 19%) (¹H NMR (300 MHz, CD₃OD) δ 1.24 (s, 3H), 1.33 (s, 3H), 1.59 (d, J=7.2 Hz, 3H), 1.64-2.00 (m, 4H), 2.82 (m, 1H), 3.25-3.29 (m, 2H), 3.40 (s, 3H), 3.65 (dd, J=11.1, 2.6 Hz, 1H), 4.45 (m, 1H), 5.77 (dd, J=9.1, 5.3 Hz, 1H), 5.92 (q, J=7.2 Hz, 1H), 6.27 (ABq, A 6AB=0.09, JAB=16.4 Hz, 2H), 7.09 (d, J=9.0 Hz, 1H), 7.19-7.26 (m, 2H), 7.29-7.42 (m, 3H), 7.46 (s, 1H), 7.64 (d, J=8.4 Hz, 1H), 8.05 (d, J=9.0 Hz, 1H); LCMS (m/z) 633, 635 [M+H]).

### Example 8: Preparation of Compound (61)

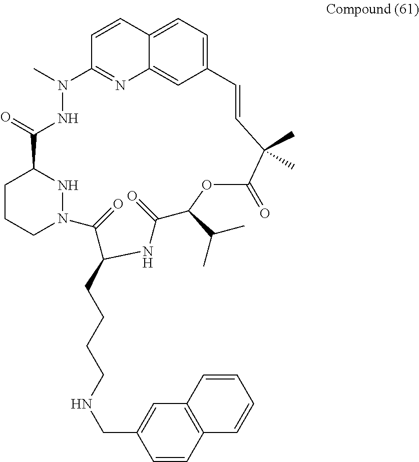

Compound (61) was prepared in a multistep process starting from commercially available methyl (2S)-6-amino-2-(benzyloxycarbonylamino)hexanoate hydrochloride as follows.

Step 1: Preparation of Methyl (2S)-2-(benzyloxycarbonylamino)-6-[tert-butoxycarbonyl(2-naphthylmethyl)amino]hexanoate (Intermediate (XIV)).

This was prepared in a multistep sequence as follows.

### Step 1a and 1b

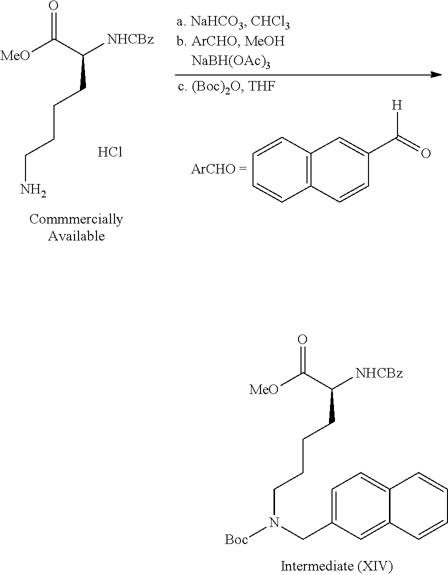

A solution of methyl (2S)-6-amino-2-(benzyloxycarbonylamino)hexanoate hydrochloride (661 mg, 2 mmol) in chloroform (20 mL) was washed with saturated sodium hydrogen carbonate solution (20 mL). The organic layer was separated and the aqueous layer was extracted with chloroform. The organic extracts were combined, washed with brine, filtered through a hydrophobic frit and the filtrate was evaporated to afford methyl (2S)-6-amino-2-(benzyloxycarbonylamino)hexanoate (612 mg, 2 mmol) (LCMS (m/z) 295 [M+H], RT=0.88 min.).

A solution of the above obtained methyl (2S)-6-amino-2-(benzyloxycarbonylamino)hexanoate (612 mg, 2 mmol) in methanol was stirred at room temperature under nitrogen. A solution of naphthalene-2-carbaldehyde (ArCHO) (312 mg, 2 mmol) was added and the reaction mixture was stirred for 2 hours. Sodium triacetoxyborohydride (NaBH(OAc)₃) (466 mg, 2.2 mmol) was added and the reaction mixture was stirred at room temperature for 1 hour. The majority of the solvent was evaporated. Saturated sodium hydrogen carbonate solution was added and the mixture was extracted with chloroform. The organic extracts were combined and washed with brine. The organic solution was filtered through a hydrophobic frit and the filtrate was evaporated to afford methyl (2S)-2-(benzyloxycarbonylamino)-6-(2-naphthylmethylamino)hexanoate (2 mmol) (LCMS (m/z) 435 [M+H], RT=1.55 min.).

Step 1c: Preparation of Methyl (2S)-2-benzyloxycarbonylamino-6-[tert-butoxycarbonyl(2-naphthylmethyl)amino]hexanoate.

A solution of methyl (2S)-2-(benzyloxycarbonylamino)-6-(2-naphthylmethylamino)hexanoate (2 mmol) in tetrahydrofuran (20 mL) was stirred at room temperature. A solution of di-tert-butyl dicarbonate (523 mg, 2.4 mmol) in tetrahydrofuran (5 mL) was added and the reaction mixture was stirred at room temperature for 3 hours. The solvent was evaporated and the residue was purified by silica gel chromatography eluting with 0-30% ethyl acetate in isohexane to afford the title compound (540 mg, 50%) as an oil (LCMS (m/z) [M+H]535, RT=3.69 min.).

Step 2: Preparation of Methyl (2S)-2-amino-6-[tert-butoxycarbonyl(2-naphthylmethyl)amino]hexanoate (Intermediate (XV)).

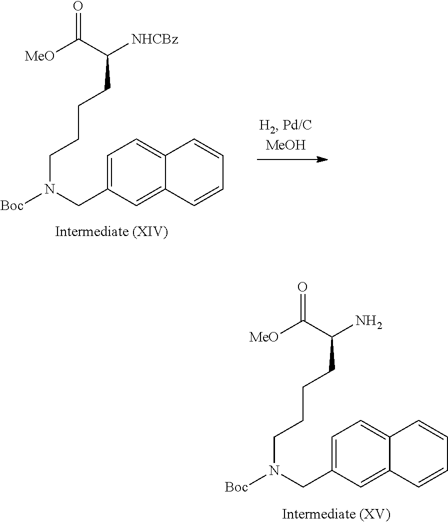

A solution of methyl (2S)-2-(benzyloxycarbonylamino)-6-[tert-butoxycarbonyl(2-naphthylmethyl)amino]hexanoate (508 mg, 0.95 mmol) in methanol (20 mL) containing 10 palladium on carbon paste (100 mg) was stirred under hydrogen at room temperature and pressure for 22 hours. The reaction mixture was filtered through a pad of Hyflo® Super-Cel filter aid and the filter pad was washed with methanol. The filtrate was evaporated to afford the title compound (350 mg, 92%) as an oil (LCMS (m/z) [M+H]401, RT=1.70 min.).

Step 3: Preparation of methyl (2S)-6-[tert-butoxycarbonyl(2-naphthylmethyl)amino]-2-[[(2S)-2-hydroxy-3-methylbutanoyl]amino]hexanoate (Intermediate (XVI)).

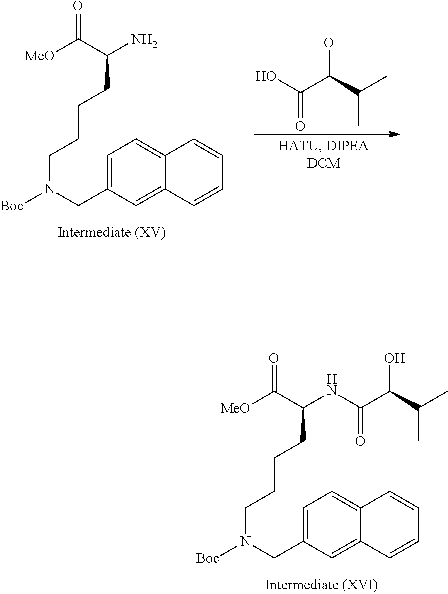

A solution of methyl (2S)-2-(benzyloxycarbonylamino)-6-(2-naphthylmethylamino)hexanoate (350 mg, 0.88 mmol) in dichloromethane (30 mL) was stirred at 0° C. under nitrogen. (2S)-2-Hydroxy-3-methylbutanoic acid (104 mg, 0.88 mmol), N,N-diisopropylethylamine (454 mg, 0.61 mL, 3.52 mmol) and was added followed by (dimethylamino)-N,N-dimethyl(3H-[1,2,3]triazolo[4,5-b]pyridin-3-yloxy)methaniminium hexafluorophosphate (500 mg, 1.32 mmol). The reaction mixture was stirred at 0° C. for 30 minutes and then at room temperature for 22 hours. The solution was diluted with dichloromethane and the solution was washed with water, saturated sodium hydrogen carbonate solution and brine. The organic layer was separated and was filtered through a hydrophobic frit and the filtrate was evaporated. The residue was purified by silica gel chromatography eluting with a gradient of 0-60% ethyl acetate in isohexane to give the title compound (332 mg, 75%) as a gum (LCMS (m/z) 501 [M+H], RT=3.22 min.).

Step 4: Preparation of (2S)-6-[tert-Butoxycarbonyl(2-naphthylmethyl)amino]-2-[[(2S)-2-hydroxy-3-methylbutanoyl]amino]hexanoic acid (Intermediate (XVII)).

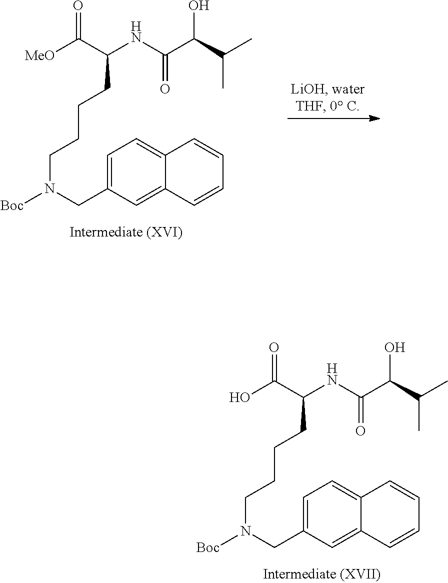 [SMILES: COC(=O)[C@H](CCCCN(Cc1ccc2ccccc2c1)C(=O)OC(C)(C)C)NC(=O)[C@@H](O)C(C)C ; CC(C)[C@H](O)C(=O)N[C@@H](CCCCN(Cc1ccc2ccccc2c1)C(=O)OC(C)(C)C)C(=O)O]

A solution of methyl (2S)-6-[tert-butoxycarbonyl(2-naphthylmethyl)amino]-2-[[(2S)-2-hydroxy-3-methylbutanoyl]amino]hexanoate (332 mg, 0.66 mmol) in tetrahydrofuran (5 mL) was stirred at −5 to −8° C. under nitrogen. A solution of lithium hydroxide hydrate (69 mg, 1.65 mmol) in water (2 mL) was added dropwise (maintain internal T<−1° C.). The reaction mixture was stirred at −5 to −1° C. for 1 hour. Cold 2 M hydrochloric acid was added dropwise to acidify the solution to pH 2-3. Brine (10 mL) was added and the mixture was extracted with ethyl acetate. The organic extracts were combined, the organic layer was filtered through a hydrophobic frit and the filtrate was evaporated. The residue was dried to afford the title compound (291 mg, 91%) as a foam (LCMS (m/z) 509 [M+Na], RT=2.95 min.).

Step 5: Preparation of tert-butyl N-[(5S)-6-[(3S)-3-[[(7-bromo-2-quinolyl)-methylamino]carbamoyl]hexahydropyridazin-1-yl]-5-[[(2S)-2-hydroxy-3-methyl-butanoyl]amino]-6-oxo-hexyl]-N-(2-naphthylmethyl)carbamate (Intermediate (XVI1l)).

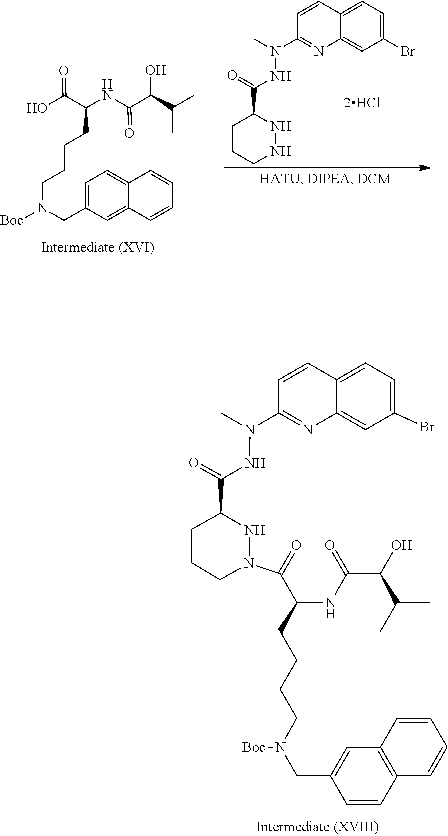 [SMILES: CC(C)[C@H](O)C(=O)N[C@@H](CCCCN(Cc1ccc2ccccc2c1)C(=O)OC(C)(C)C)C(=O)O ; CN(NC(=O)[C@@H]1CCCNN1)c1ccc2ccc(Br)cc2n1 ; CC(C)[C@H](O)C(=O)N[C@@H](CCCCN(Cc1ccc2ccccc2c1)C(=O)OC(C)(C)C)C(=O)N1CCC[C@@H](C(=O)NN(C)c2ccc3ccc(Br)cc3n2)N1]

A suspension of (3S)-N′-(7-bromo-2-quinolyl)-N′-methyl-hexahydropyridazine-3-carbohydrazide dihydrochloride (as previously described in Example 7 (Step 1; Intermediate (IX)) (291 mg, 0.6 mmol) in dichloromethane (20 mL) was stirred at 0° C. under nitrogen. (2S)-6-[tert-butoxycarbonyl(2-naphthylmethyl)amino]-2-[[(2S)-2-hydroxy-3-methylbutanoyl]amino]hexanoic acid (260 mg, 0.6 mmol) and N,N-diisopropylethylamine (0.52 mL, 3.0 mmol) giving a solution. (Dimethylamino)-N,N-dimethyl(3H-[1,2,3]triazolo[4,5-b]pyridin-3-yloxy)methaniminium hexafluorophosphate (319 mg, 0.84 mmol) was added and the reaction mixture was stirred at 0° C. for 30 minutes and then at room temperature for 2 hours. The reaction mixture was diluted with dichloromethane and the solution was washed with water, saturated sodium hydrogen carbonate solution and brine. The organic layer was separated and was filtered through a hydrophobic frit and the filtrate was evaporated. The residue was purified by silica gel chromatography eluting with a gradient of 0-50% acetone in isohexane. The residue was triturated with diethyl ether/isohexane (1:2, 15 mL), the solvent was removed and the residue was dried to give the title compound (266 mg, 53%) as a foam (LCMS (m/z) 832, 834 [M+H], RT=3.57 min.).

### Step 6: Preparation of Compound (61).

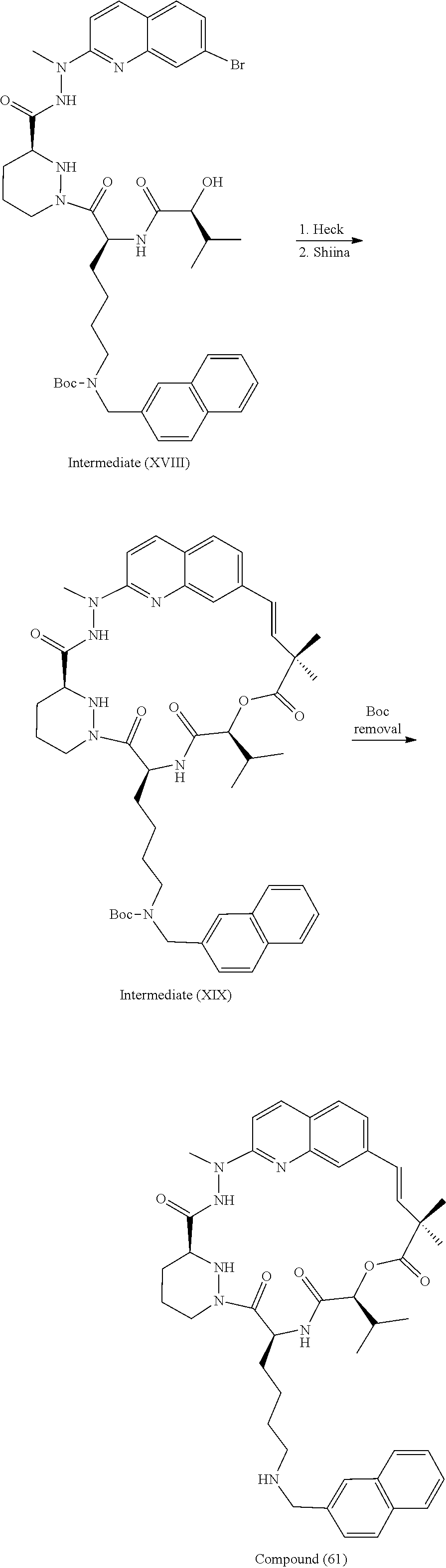

tert-Butyl-N-[(5S)-6-[(3S)-3-[[(7-bromo-2-quinolyl)-methylamino]carbamoyl]hexahydropyridazin-1-yl]-5-[[(2S)-2-hydroxy-3-methyl-butanoyl]amino]-6-oxo-hexyl]-N-(2-naphthylmethyl)carbamate (from Step 5) was further elaborated by (i) a coupling reaction with 2,2-dimethyl-3-butenoic acid using methods already described in Example 7 (Step 5) and then (ii) a ring closing esterification/macrolactonisation reaction using methods already described in Example 7 (Step 6) followed by removal of the Boc protecting group using methods already described in Example 7 (Step 2) to provide Compound (61).

### Example 9: Preparation of Compound 10

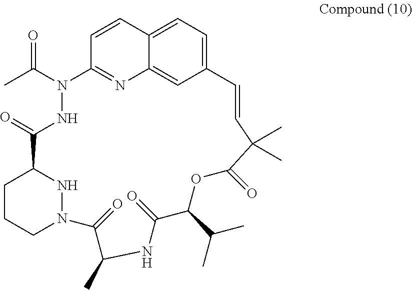 [SMILES: CC(=O)N1NC(=O)[C@@H]2CCCN(N2)C(=O)[C@H](C)NC(=O)[C@H](C(C)C)OC(=O)C(C)(C)/C=C/c2ccc3ccc1nc3c2]

Compound (10) was prepared from commercially available (7-Bromo-2-quinolyl)hydrazine as described below.

Step 1: Preparation of (7-bromo-2-quinolyl)hydrazine (Intermediate (XX)).

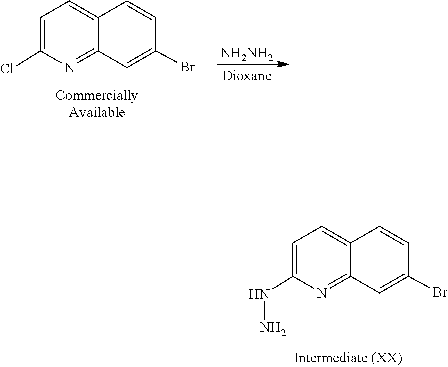

Hydrazine hydrate (1.5 ml, 30 mmol) was added to a suspension of the commercially available 7-bromo-2-chloroquinoline (1.22 g, 5 mmol) in dioxane (10 ml) and the reaction was stirred at 90° C. for 6 hours. After cooling, water and ethyl acetate was added and the phases separated. The organic layer was passed through a hydrophobic frit and the solvent evaporated to afford the crude title compound as a pale yellow solid (1.07 g) (¹H NMR (300 MHz, CDCl₃) δ 4.1 (2H, s), 6.05 (1H, s), 6.8 (1H, d), 7.35 (1H, d), 7.5 (1H, d), 7.8 (1H, d), 7.95 (1H, s)).

Step2: Preparation of di-tert-butyl(3S)-3-[[(7-bromo-2-quinolyl)amino]carbamoyl]hexahydropyridazine-1,2-dicarboxylate (Intermediate (XXI)).

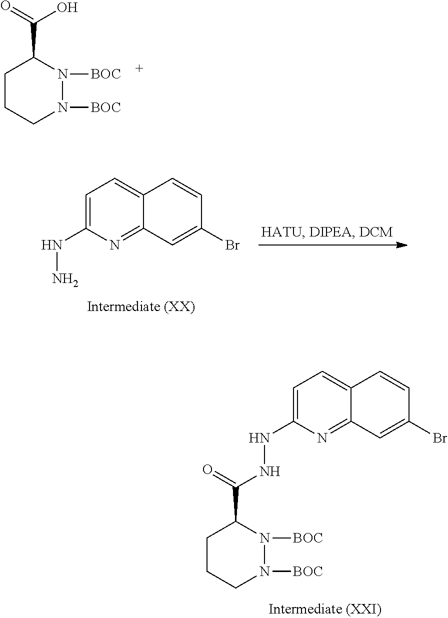 [SMILES: CC(C)(C)OC(=O)N1CCC[C@@H](C(=O)O)N1C(=O)OC(C)(C)C ; NNc1ccc2ccc(Br)cc2n1 ; CC(C)(C)OC(=O)N1CCC[C@@H](C(=O)NNc2ccc3ccc(Br)cc3n2)N1C(=O)OC(C)(C)C]

[(Dimethylamino)-1H-1,2,3-triazolo-[4,5-b]pyridin-1-ylmethylene]-N-methylmethanaminium hexafluorophosphate N-oxide (461 mg, 1.21 mmol) was added to a suspension of the (3S)-1,2-bis(tert-butoxycarbonyl)hexahydropyridazine-3-carboxylic acid (308 mg, 0.93 mmol), (7-bromo-2-quinolyl)hydrazine (222 mg, 0.93 mmol) and N,N-diisopropylethylamine (0.49 ml, 2.8 mmol) in dichloromethane (15 ml) and the reaction was stirred at room temperature for 1 hour. The reaction was washed with saturated aqueous sodium bicarbonate, passed through a hydrophobic frit and the solvent evaporated. The crude material was purified by silica chromatography (0 to 50% ethyl acetate/isohexane) to afford the title compound as a yellow foam (502 mg) (LCMS M+H⁺=550, 552, C₂₄H₃₂BrN₅O₅ requires 549, 551; RT=3.15 mins.).

Step 3: Preparation of di-tert-butyl(3S)-3-[[acetyl-(7-bromo-2-quinolyl)amino]carbamoyl]hexahydropyridazine-1,2-dicarboxylate (Intermediate (XXII)).

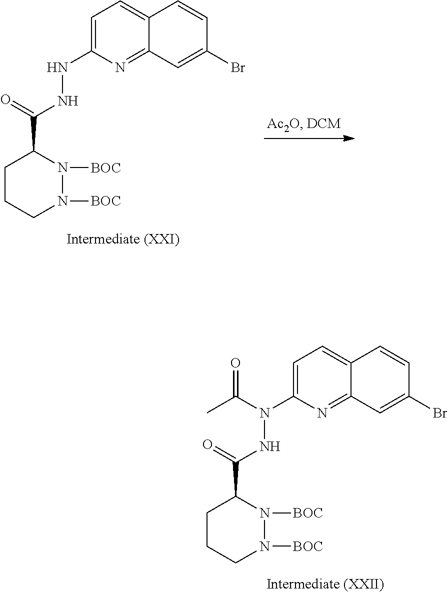 [SMILES: CC(C)(C)OC(=O)N1CCC[C@@H](C(=O)NNc2ccc3ccc(Br)cc3n2)N1C(=O)OC(C)(C)C ; CC(=O)N(NC(=O)[C@@H]1CCCN(C(=O)OC(C)(C)C)N1C(=O)OC(C)(C)C)c1ccc2ccc(Br)cc2n1]

Di-tert-butyl(3S)-3-[[(7-bromo-2-quinolyl)amino]carbamoyl]hexahydropyridazine-1,2-dicarboxylate (502 mg, 0.91 mmol) was dissolved in dichloromethane (20 ml) and acetic anhydride (0.17 ml, 1.82 mmol) was added. The reaction was stirred for 7 hours. To the solution was added saturated aqueous sodium bicarbonate and the phases separated, the organic layer was passed through a hydrophobic frit and the solvent evaporated to afford the title compound as a yellow foam (551 mg) (LCMS M+H⁺=592, 594, C₂₆H₃₄BrN₅O₆ requires 591, 593; RT=3.58 mins.).

### Step 4: Preparation of Compound (10).

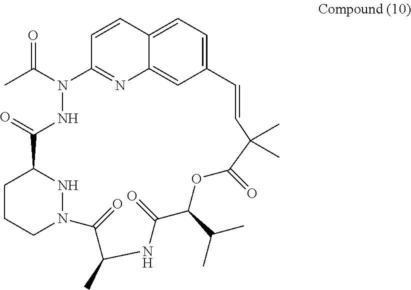 [SMILES: CC(=O)N1NC(=O)[C@@H]2CCCN(N2)C(=O)[C@H](C)NC(=O)[C@H](C(C)C)OC(=O)C(C)(C)/C=C/c2ccc3ccc1nc3c2]

The final steps synthesis of Compound (10) were completed in a manner similar to Example 1, steps 3 to 5. Compound (10) (LCMS M+H⁺=579, C₃₀H₃₈N₆O₆ requires 578; RT=1.96 mins.).

### Example 10: Preparation of Compound 24

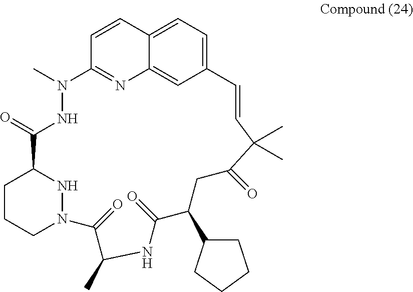 [SMILES: C[C@@H]1NC(=O)[C@H](C2CCCC2)CC(=O)C(C)(C)/C=C/c2ccc3ccc(nc3c2)N(C)NC(=O)[C@@H]2CCCN(N2)C1=O]

Step 1: Preparation of ethyl 2-iodo-2-cyclopentyl-acetate (Intermediate (XXIII)).

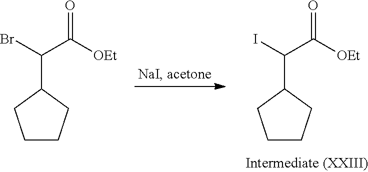 [SMILES: CCOC(=O)C(Br)C1CCCC1 ; CCOC(=O)C(I)C1CCCC1]

To a solution of ethyl 2-bromo-2-cyclopentyl-acetate (13.5 g, 57.4 mmol) in acetone (180 ml) was added sodium iodide (26 g, 172.3 mmol) and the mixture was heated at reflux for 18 hours. After cooling the solvent was evaporated and the residue was partitioned between diethyl ether and 10% sodium thiosulfite. The phases were separated, the organic layer was dried (magnesium sulphate) and the solvent evaporated to afford the title compound as a yellow oil (9 g) (¹H NMR (300 MHz, CDCl₃) δ 1.15 (1H, m), 1.25 (3H, t), 1.3 (1H, m), 1.5-2.05 (7H, m), 2.45 (1H, m), 4.15 (2H, m)).

Step 2: Preparation of ethyl 2-cyclopentyl-5,5-dimethyl-4-oxo-hept-6-enoate (Intermediate (XXIV)).

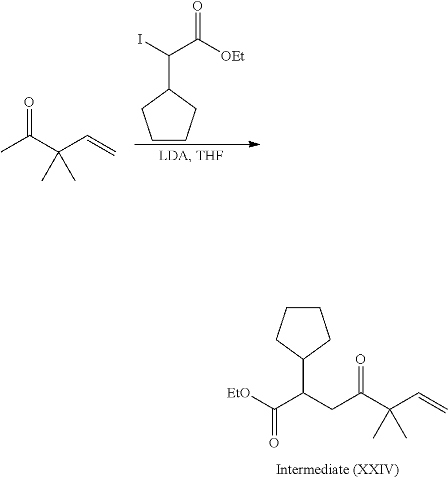

To a solution of N,N-diisopropylamine (LDA) (2.8 ml, 19.6 mmol) in tetrahydrofuran (20 ml) at −15° C. under N₂ was added n-butyllithium (7.5 ml, 18.8 mmol, 2.5M in hexanes) dropwise over 45 minutes. The reaction was stirred for 15 minutes before cooling to −78° C. To this was added 3,3-dimethylpent-4-en-2-one (Nakagawa-Goto et al., Synlett, 11, 1555-1558 (2011)) (2 g, 17.9 mmol) in tetrahydrofuran (10 ml) over 15 minutes. The reaction was stirred for 30 minutes before the dropwise addition of the ethyl 2-iodo-2-cyclopentyl-acetate (5.3 g, 19.6 mmol) in tetrahydrofuran (10 ml) over 15 minutes. The reaction was stirred for 30 minutes at −78° C. before warming to room temperature. The reaction was quenched with saturated aqueous ammonium chloride and diluted with diethyl ether. The phases were separated and the organic layer dried with a hydrophobic frit. The solvent was evaporated and the residue purified by silica chromatography (isohexane/ethyl acetate, 20:1) to afford the title compound as a yellow oil (1.2 g) (¹H NMR (300 MHz, CDCl₃) δ 1.25 (9H, m), 1.4-2.1 (9H, m), 2.6 (2H, m), 2.95 (1H, dd), 4.15 (2H, m), 5.15 (2H, m), 5.95 (1H, dd)).

Step 3: Preparation of Ethyl (E)-7-[2-[(tert-butoxycarbonylamino)-methyl-amino]-7-quinolyl]-2-cyclopentyl-5,5-dimethyl-4-oxo-hept-6-enoate (Intermediate (XXV)).

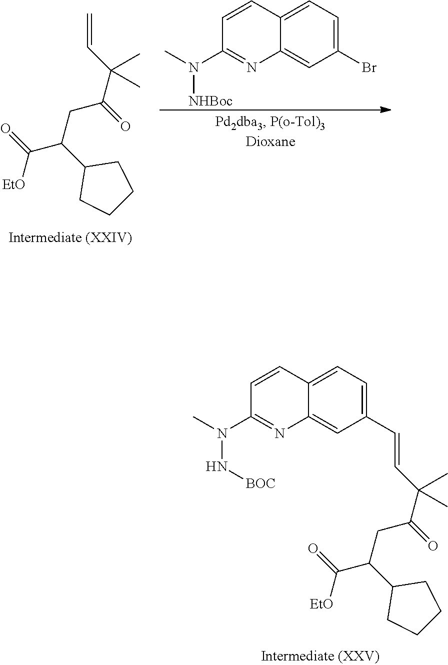

To a stirred solution of tert-butyl-N-[(7-bromo-2-quinolyl)-methyl-amino]carbamate (Example 1, step 1, Intermediate (I)) (1.45 g, 4.12 mmol) and ethyl 2-cyclopentyl-5,5-dimethyl-4-oxo-hept-6-enoate (1 g, 3.75 mmol) in dioxane (20 mL) was added tri-(o-tolyl)phosphine (456 mg, 1.5 mmol) and dicyclohexylmethylamine (1.3 ml, 5.99 mmol). The reaction mixture was degassed with nitrogen for 10 minutes. Palladium acetate (336 mg, 1.5 mmol) was added and the reaction mixture was heated in a microwave vial at 140° C. for 4 hours. The reaction was cooled to room temperature and the mixture was filtered through celite and the filtrate was evaporated. The residue was purified by silica chromatography (isohexane/ethyl acetate, 2:1) to afford the title compound as a yellow oil (LCMS M+H⁺=538, C₃₁H₄₃N₃O₅ requires 537; RT=2.98 mins.).

Step 4: Preparation of (E)-7-[2-[(tert-butoxycarbonylamino)-methyl-amino]-7-quinolyl]-2-cyclopentyl-5,5-dimethyl-4-oxo-hept-6-enoic acid (Intermediate (XXVI)).

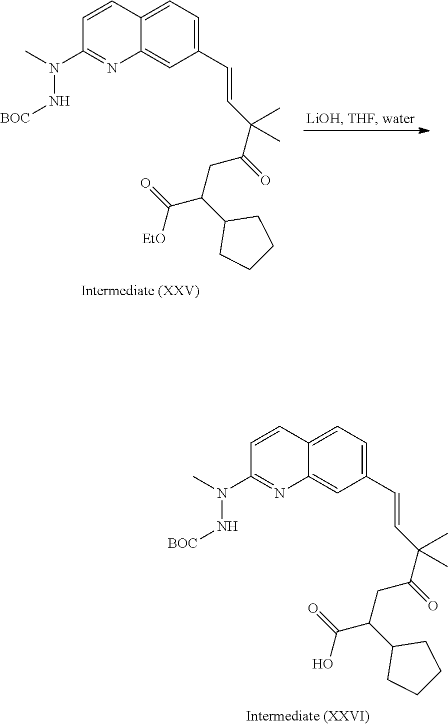

Lithium hydroxide hydrate (859 mg, 20.4 mmol) was added to a solution of the ethyl (E)-7-[2-[(tert-butoxycarbonylamino)-methyl-amino]-7-quinolyl]-2-cyclopentyl-5,5-dimethyl-4-oxo-hept-6-enoate (1.1 g, 2.04 mmol) in tetrahydrofuran (20 ml) and water (4 ml) and the reaction was stirred at 50° C. for 20 hours. The solvent was evaporated and the residue dissolved in water. This was acidified (2 M HCl, pH˜2) and the aqueous layer extracted with ethyl acetate (3×). The combined organics were passed through a hydrophobic frit and the solvent evaporated to afford the title compound as a yellow foam (780 mg) (LCMS M+H⁺=510, C₂₉H₃₉N₃O₅ requires 509; RT=2.3 mins.).

Step 5: Prepation of 2,2,2-Trichloroethyl (3S)-1-[(25)-2-[[(E)-7-[2-[(tert-butoxycarbonylamino)-methyl-amino]-7-quinolyl]-2-cyclopentyl-5,5-dimethyl-4-oxo-hept-6-enoyl]amino]propanoyl]hexahydropyridazine-3-carboxylate (Intermediate (XXVII)).

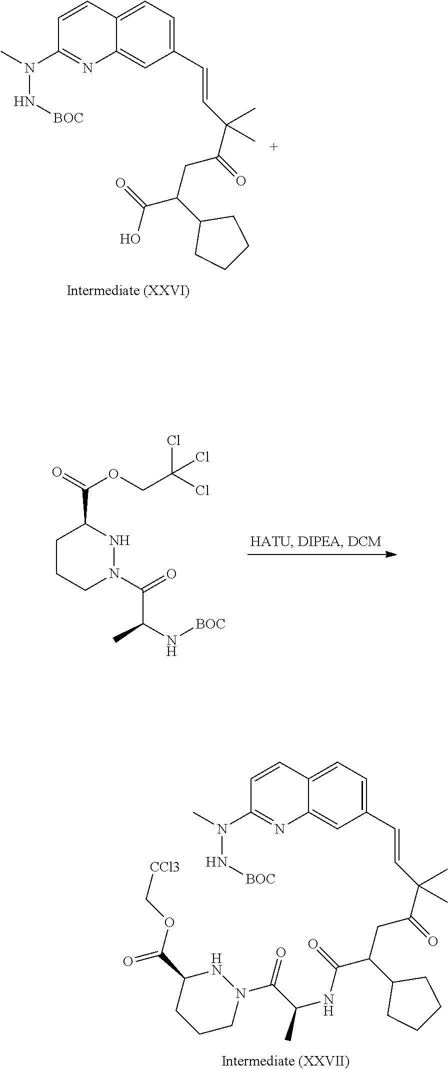

4 M HCl in dioxane (3.7 ml) was added to a solution of the 2,2,2-trichloroethyl (3S)-1-[(25)-2-(tert-butoxycarbonylamino)propanoyl]hexahydropyridazine-3-carboxylate (Steadman et al., J. Med. Chem., 60, 1000-1017 (2017)) (700 mg, 1.62 mmol) in dichloromethane (10 ml) and the reaction was stirred for 1 hour. The solvent was evaporated to afford a white solid. To this was added the (E)-7-[2-[(tert-butoxycarbonylamino)-methyl-amino]-7-quinolyl]-2-cyclopentyl-5,5-dimethyl-4-oxo-hept-6-enoic acid (750 mg, 1.47 mmol) in dichloromethane (7 ml), followed by N,N-diisopropylethylamine (1.06 ml, 5.89 mmol) and [(dimethylamino)-1H-1,2,3-triazolo-[4,5-b]pyridin-1-ylmethylene]-N-methylmethanaminium hexafluorophosphate N-oxide (784 mg, 2.06 mmol). The reaction was stirred at room temperature for 18 hours. The reaction was washed with 0.5 M hydrochloric acid, saturated aqueous sodium bicarbonate, saturated aqueous brine, passed through an hydrophobic frit and the solvent evaporated. The crude material was purified by silica chromatography (25 to 33% ethyl acetate/isohexane) to afford the title compound as a white solid (700 mg) (LCMS M+H⁺=825, C₃₉H₅₃Cl₃N₆O₇ requires 824; RT=2.79 min., 2.84 min. (mixture of diastereoisomers)).

Step 6: Preparation of (3S)-1-[(25)-2-[[(E)-7-[2-[Amino(methyl)amino]-7-quinolyl]-2-cyclopentyl-5,5-dimethyl-4-oxo-hept-6-enoyl]amino]propanoyl]hexahydropyridazine-3-carboxylic acid (Intermediate (XXVIII)).

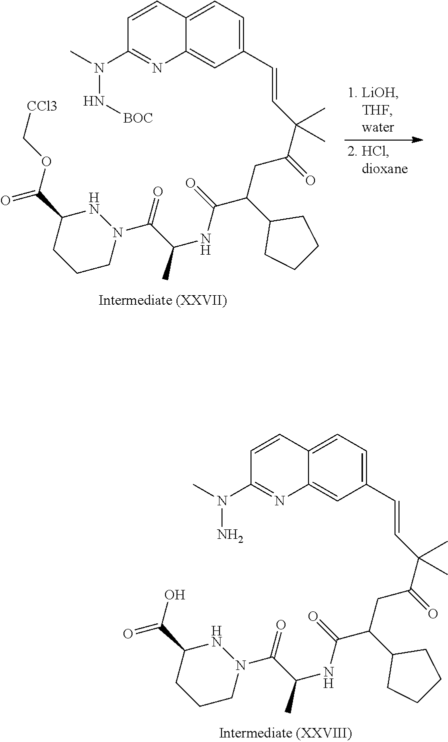

Lithium hydroxide hydrate (54 mg, 1.28 mmol) was added to a solution of the 2,2,2-trichloroethyl (3S)-1-[(25)-2-[[(E)-7-[2-[(tert-butoxycarbonylamino)-methyl-amino]-7-quinolyl]-2-cyclopentyl-5,5-dimethyl-4-oxo-hept-6-enoyl]amino]propanoyl]hexahydropyridazine-3-carboxylate (700 mg, 0.85 mmol) in tetrahydrofuran (12 ml) and water (3 ml) and the reaction was stirred at room temperature for 15 minutes. The mixture was acidified (2 M HCl, 3-4 pH) and solid sodium chloride was added. The mixture was extracted with ethyl acetate (2×) and the combined organics passed through a hydrophobic frit and the solvent evaporated. The crude was dissolved in dichloromethane (2 ml) and 4 M HCl in dioxane (2 ml) was added. The reaction was stirred for 30 minutes and the solvent evaporated to afford the title compound which was used as is in the next step (LCMS M+H⁺=593, C₃₂H₄₄N₆O₅ requires 592; RT=1.64 mins.).

### Step 7: Preparation of Compound (24).

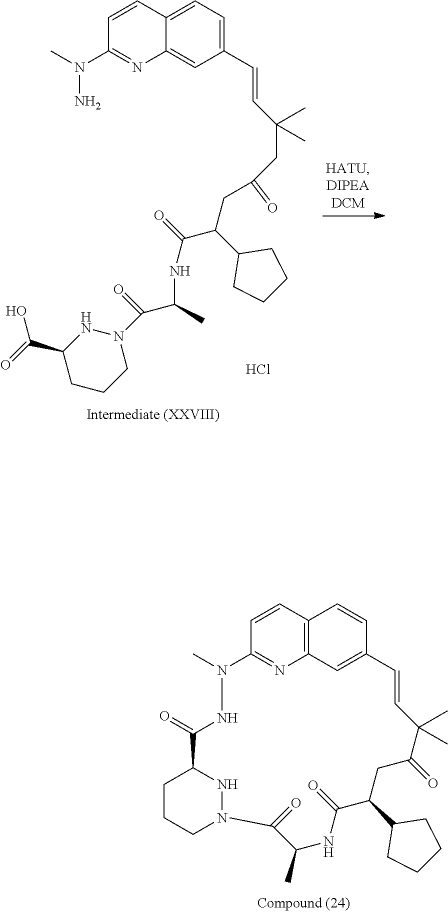 [SMILES: C[C@H](NC(=O)C(CC(=O)CC(C)(C)/C=C/c1ccc2ccc(N(C)N)nc2c1)C1CCCC1)C(=O)N1CCC[C@@H](C(=O)O)N1 ; Cl ; C[C@@H]1NC(=O)[C@H](C2CCCC2)CC(=O)C(C)(C)/C=C/c2ccc3ccc(nc3c2)N(C)NC(=O)[C@@H]2CCCN(N2)C1=O]

(3S)-1-[(25)-2-[[(E)-7-[2-[Amino(methyl)amino]-7-quinolyl]-2-cyclopentyl-5,5-dimethyl-4-oxo-hept-6-enoyl]amino]propanoyl]hexahydropyridazine-3-carboxylic acid (Intermediate (XXVIII)) as its dihydrochloride salt (0.85 mmol) was dissolved in anhydrous DCM (283 ml) and DIPEA (0.76 ml, 4.75 mmol) was added. HATU (453 mg, 1.2 mmol) was added with stirring under nitrogen. The reaction mixture was stirred at room temperature overnight and then concentrated to ˜50 ml under reduced pressure. Washed with 0.5 M HCl, saturated sodium bicarbonate and then dried by passing through a hydrophobic frit. The solvent was evaporated under reduced pressure and the residue was purified by silica gel chromatography (eluted with isohexane: acetone 3:2) to give the crude product (80 mg) nd further purified by silica gel chromatography (eluted with ethyl acetate) to give the crude product (60 mg) (¹H NMR (300 MHz, CD₃OD) 8.1 (d, 1H, J=9 Hz), 7.7 (d, 1H, J=9 Hz), 7.65 (m, 1H), 7.4 (m, 1H), 7.15 (d, 1H, 9 Hz), 6.4 (d, 1H, J=16 Hz), 6.15 (d, 1H, J=16 Hz), 6.0 (m, 1H), 4.5 (m, 1H), 3.7 (m, 1H), 3.45 (s, 3H), 2.8 (m, 2H), 2.4 (m, 1H), 2.0-1.2 (m, 12H), 1.6 (d, 3H, J=7 Hz), 1.55 (s, 3H), 1.3 (s, 3H); LC m/z[M+H]575).

### Example 11: Preparation of Compound (25) and Compound (82)

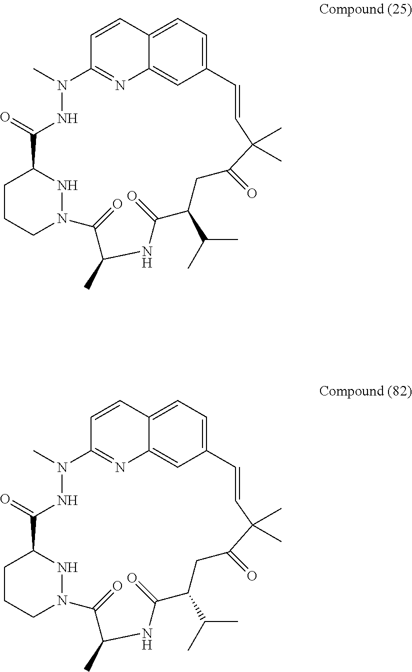 [SMILES: CC(C)[C@@H]1CC(=O)C(C)(C)/C=C/c2ccc3ccc(nc3c2)N(C)NC(=O)[C@@H]2CCCN(N2)C(=O)[C@H](C)NC1=O ; CC(C)[C@H]1CC(=O)C(C)(C)/C=C/c2ccc3ccc(nc3c2)N(C)NC(=O)[C@@H]2CCCN(N2)C(=O)[C@H](C)NC1=O]

Preparation of Starting Material ethyl 2-isopropyl-5,5-dimethyl-4-oxo-hept-6-enoate (Intermediate (XXIX)).

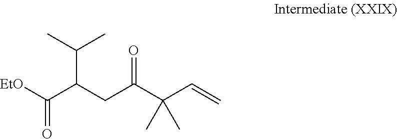 [SMILES: C=CC(C)(C)C(=O)CC(C(=O)OCC)C(C)C]

Ethyl 2-isopropyl-5,5-dimethyl-4-oxo-hept-6-enoate was prepared in a manner similar to that of Example 10 (step 2) above, by substituting ethyl 2-iodo-2-cyclopentyl-acetate for ethyl 2-iodo-3-methyl-butanoate (WO 2007/096395 (Nycomed GmbH)) (¹H NMR (300 MHz, CDCl₃) δ 0.9 (9H, m), 1.25 (6H, m), 1.95 (1H, m), 2.5 (1H, dd), 2.7 (1H, m), 2.95 (1H, dd), 4.15 (2H, m), 5.15 (2H, m), 5.9 (1H, dd)).

The remaining preparation of Compounds (25) and Compound (82) were completed in a manner similar to that of Example 10 (steps 3 to 7 inclusive). The two diastereomeric products were separated to afford:
Compound (25): LCMS M+H⁺=549, C₃₀H₄₀N₆O₄ requires 548; RT=1.78 mins.
Compound (82): LCMS M+H⁺=549, C₃₀H₄₀N₆O₄ requires 548; RT=1.65 mins.
NMR (300 MHz; CD₃OD) δ 8.1 (d, 1H), 8.0 (m, 1H), 7.7 (m, 1H), 7.25 (d, 1H), 7.1 (d, 1H), 6.6 (d, 1H), 6.2 (d, 1H), 5.85 (m, 1H), 4.5 (m, 1H), 3.9 (m, 1H), 3.4 (s, 3H), 2.8 (m, 2H), 2.4 (m, 1H), 2.1 (m, 1H), 1.9-1.7 (m, 5H), 1.5 (s, 3H), 1.35 (m, 3H), 1.35 (m, 3H), 1.3 (m, 3H), 1.0 (d, 3H) (multiple conformers).

### Example 12: Preparation of Compound (7) and Compound (8)

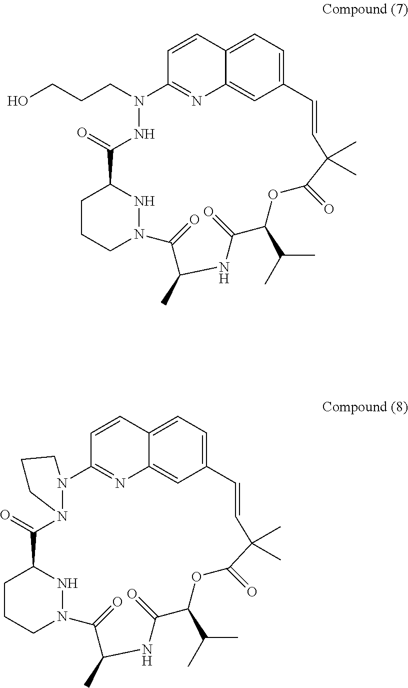 [SMILES: CC(C)[C@@H]1OC(=O)C(C)(C)/C=C/c2ccc3ccc(nc3c2)N(CCCO)NC(=O)[C@@H]2CCCN(N2)C(=O)[C@H](C)NC1=O ; CC(C)[C@@H]1OC(=O)C(C)(C)/C=C/c2ccc3ccc(nc3c2)N2CCCN2C(=O)[C@@H]2CCCN(N2)C(=O)[C@H](C)NC1=O]

Step 1: Preparation of tert-butyl N-[3-[(4-methoxyphenyl)methoxy]propylamino]carbamate (Intermediate XXX).

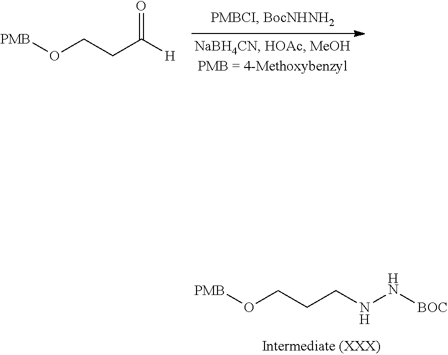

Commercially available 3-[(4-methoxyphenyl)methoxy]propanal (1.05 g, 5.44 mmol) and tert-butyloxycarbonylhydrazine (BocNHNH₂) (720 mg, 5.44 mmol) were dissolved in methanol (30 ml). To this was added acetic acid (0.62 ml, 10.88 mmol) and sodium cyanoborohydride (342 mg, 5.44 mmol) and the reaction was stirred at room temperature for 18 hours. Saturated aqueous sodium bicarbonate was added and the resultant mixture extracted with dichloromethane (2×). The organic layer was passed through a hydrophobic frit and the solvent evaporated. This was purified by silica chromatography (0 to 40% ethyl acetate/isohexane) to afford the title compound as a solid (548 mg) (¹H NMR (300 MHz, CDCl₃) δ 1.5 (9H, s), 1.8 (2H, m), 2.95 (2H, m), 3.5 (2H, t), 3.8 (3H, s), 4.0 (1H, br s), 4.45 (2H, s), 6.05 (1H, br s), 6.9 (2H, d), 7.3 (2H, d)).

Contrary to the reaction scheme hereinabove, PMBCI is not, in fact, required.

Step 2: Preparation of tert-Butyl N-[(7-bromo-2-quinolyl)-[3-[(4-methoxyphenyl)methoxy]propyl]amino]carbamate (Intermediate (XXXI)).

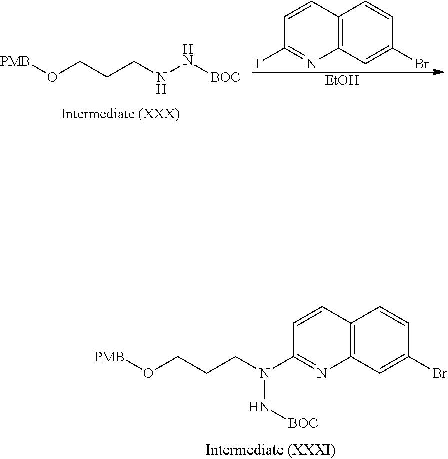

A mixture of 2-iodo-7-bromoquinoline (589 mg, 1.76 mmol) and tert-butyl N-[3-[(4-methoxyphenyl)methoxy]propylamino]carbamate (548 mg, 1.76 mmol) in ethanol (20 ml) was heated at reflux for 18 hours. The solvent was evaporated and the crude material purified by silica chromatography (0 to 20% ethyl acetate/isohexane) to afford the title compound as a yellow oil (597 mg) (¹H NMR (300 MHz, CDCl₃) δ 1.55 (10H, m), 2.05 (2H, m), 3.05 (2H, t), 3.8 (31H, s), 4.05 (2H, br m), 4.4 (2H, s), 6.85 (2H, d), 7.05 (1H, d), 7.2 (2H, d), 7.35 (1H, d), 7.45 (1H, d), 7.85 (1H, d), 7.9 (1H, s)).

### Step 3: Preparation of Compound (7) and Compound (8).

The synthesis of Compound (7) and Compound (8) was completed in a manner similar to that described in Example 5 (steps 3 to 7). The 4-methoxybenzyl (PMB) protecting group was removed in the 4 M HCl deprotection step (c.f. Example 5 (step 6)). The final macrocyclisation step (c.f. Example 5 (step 7)) gave Compound 7 and Compound 8:
Compound (7): LCMS M+H⁺=595, C₃₁H₄₂N₆O₆ requires 594; RT=1.89 mins.
Compound (8): LCMS M+H⁺=577, C₃₁H₄₀N₆O₅ requires 576; RT=1.90 mins.

### Example 13: Preparation of Compound (23)

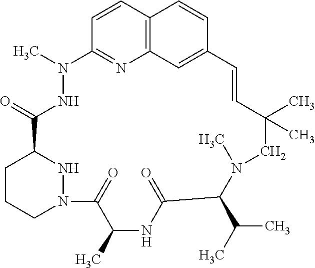 [SMILES: CC(C)[C@H]1C(=O)N[C@@H](C)C(=O)N2CCC[C@H](N2)C(=O)NN(C)c2ccc3ccc(cc3n2)/C=C/C(C)(C)CN1C]

This compound was prepared following the procedure described for Compound (20) in Example 14 by using N-Boc-N-methyl valine in step 2 instead of N-Boc-valine (¹H NMR (CDCl3): 8.1 (m, 1H), 7.8 (m, 1H), 7.65 (m, 1H), 7.3 (m, 1H), 7.15 (m, 1H), 6.7 (m, 1H), 6.45 (m, 1H), 5.8, 5.1 (m, 1H), 4.3 (m, 1H), 3.65 (m, 1H), 3.45, 3.4 (2s, 3H), 3.0-2.8 (m, 2H), 2.75, 2.6 (2 s, 3H), 2.5 (m, 1H), 2.2 (m, 2H), 1.8 (m, H), 1.5, 1.0 (2d, 3H), 1.3, 1.2 (2s, 3H), 1,15 (s, 3H), 0.95, 0.8 (2m, 6H)).

### Example 14: Preparation of Compound (20)

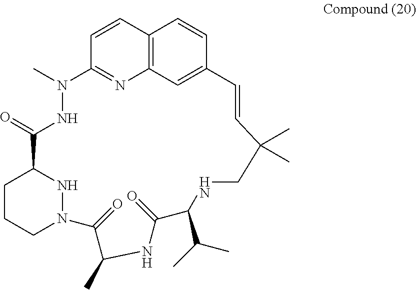 [SMILES: CC(C)[C@@H]1NCC(C)(C)/C=C/c2ccc3ccc(nc3c2)N(C)NC(=O)[C@@H]2CCCN(N2)C(=O)[C@H](C)NC1=O]

Step 1: Preparation of (3S)-1-[(2S)-2-aminopropanoyl]-N′-(7-bromo-2-quinolyl)-N′-methyl-hexahydropyridazine-3-carbohydrazide (Intermediate (XXXII)).

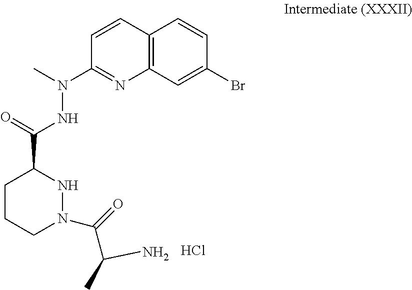 [SMILES: C[C@H](N)C(=O)N1CCC[C@@H](C(=O)NN(C)c2ccc3ccc(Br)cc3n2)N1 ; Cl]

Intermediate (XXXII) was prepared in a manner analogous to that described in step 3 of Example 1.

Step 2: Preparation of tert-butyl N-[(1S)-1-[[(1S)-2-[(3S)-3-[[(7-bromo-2-quinolyl)-methyl-amino]carbamoyl]hexahydropyridazin-1-yl]-1-methyl-2-oxo-ethyl]carbamoyl]-2-methyl-propyl]carbamate (Intermediate (XXXIII)).

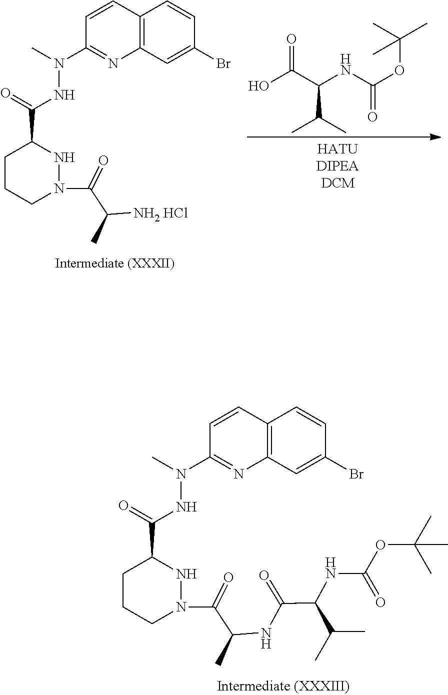

To a solution of the (3S)-1-[(2S)-2-aminopropanoyl]-N′-(7-bromo-2-quinolyl)-N′-methyl-hexahydropyridazine-3-carbohydrazide (Intermediate (XXXII)) hydrochloride (1.0 g, 2.3 mmol) in DCM (60 ml) at 0° C. was added Boc-N-methyl-L-valine (0.58 g, 2.53 mmol), DIPEA (2.0 ml, 11.5 mmol) and HATU (1.2 g, 3.16 mmol). The reaction mixture was allowed to warm to room temperature and stirred at room temperature overnight. The reaction mixture was washed with saturated ammonium chloride and saturated sodium bicarbonate. The organic layer was dried by passing through a hydrophobic frit and then evaporated to dryness. The product was purified by silica gel chromatography by elution with 0 to 30% (18:80:2 methanol: ethyl acetate: 880 ammonia) and ethyl acetate. The product was isolated as a colourless oil (1.3 g).

Step 3. Preparation of (2S)-2-amino-N-[(1S)-2-[(3S)-3-[[(7-bromo-2-quinolyl)-methyl-amino]carbamoyl]hexahydropyridazin-1-yl]-1-methyl-2-oxo-ethyl]-3-methyl-butanamide (Intermediate (XXXIV)).

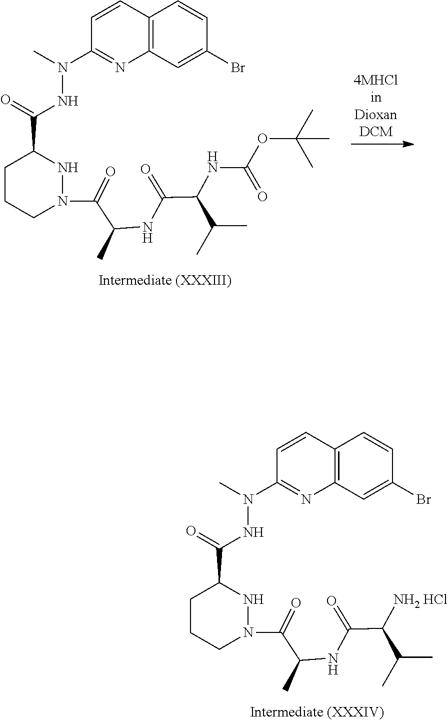

Tert-butyl-N-[(1S)-1-[[(1S)-2-[(3S)-3-[[(7-bromo-2-quinolyl)-methyl-amino]carbamoyl]hexahydropyridazin-1-yl]-1-methyl-2-oxo-ethyl]carbamoyl]-2-methyl-propyl]carbamate (Intermediate (XXXIII)) (1.03 g, 1.62 mmol) was dissolved in DCM (80 ml) and 4 M HCl in dioxan (10 ml, 40 mmol) was added with stirring. The reaction mixture was stirred at room temperature overnight. The solvent was evaporated under reduced pressure to give the hydrochloride salt of the product as a white solid (1.01 g).

Step 4: Preparation of (2S)-N-[(1S)-2-[(3S)-3-[[(7-bromo-2-quinolyl)-methyl-amino]carbamoyl]hexahydropyridazin-1-yl]-1-methyl-2-oxo-ethyl]-2-(2,2-dimethylbut-3-enylamino)-3-methyl-butanamide (Intermediate (XXXV)).

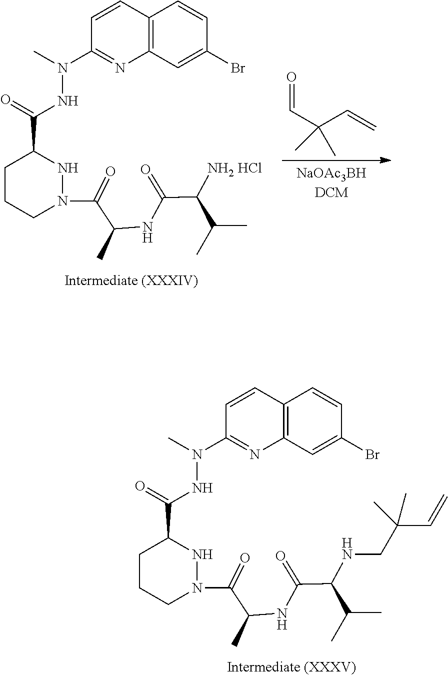

To a solution of Intermediate (XXXIV) (600 mg, 1.05 mmol) in THF (20 ml) was added 2,2-dimethylbut-3-enal (100 mg, 1.02 mmol) with stirring. Sodium triacetoxyborohydride (445 mg, 2.1 mmol) was added and the reaction mixture stirred at room temperature overnight. The solvent was evaporated under reduced pressure and the residue taken up in DCM/aqueous sodium bicarbonate. After separation, the aqueous phase was extracted with DCM, the DCM extracts combined and dried by passing through a hydrophobic frit. The solvent was evaporated under reduced pressure and the residue purified by silica gel chromatography eluting with 0 to 30% (18:80:2 methanol: ethyl acetate: 880 aqueous ammonia) in ethyl acetate to give the required product as a colourless gum (75 mg).

### Preparation of Compound (20).

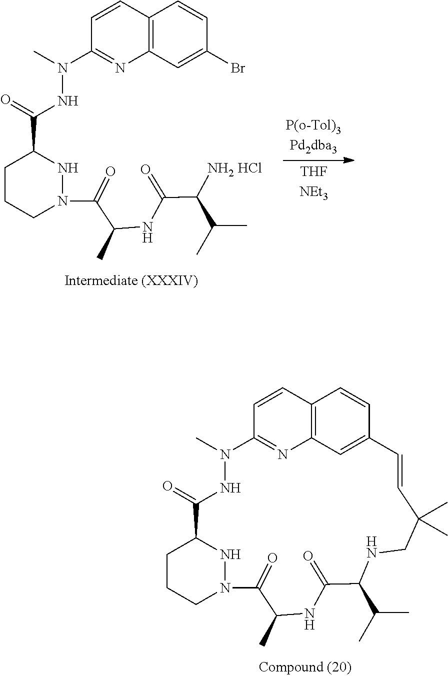

(2S)-N-[(1S)-2-[(3S)-3-[[(7-bromo-2-quinolyl)-methyl-amino]carbamoyl]hexahydropyridazin-1-yl]-1-methyl-2-oxo-ethyl]-2-(2,2-dimethylbut-3-enylamino)-3-methyl-butanamide (Intermediate (XXXV)) (118 mg, 0.19 mmol) was dissolved in THF (120 ml) and P(o-tol)₃ (12 mg, 0.039 mmol) and triethylamine (80 uL, 0.57 mmol) were added. The reaction mixture was purged with nitrogen and Pd₂dba₃ (17 mg, 0.019 mmol) was added. The reaction mixture was heated at reflux for 3 hours. Additional aliquots of P(o-tol)₃ (13 mg, 0.19 mmol) Pd₂dba3 (18 mg, 0.019 mmol) were added and the reaction mixture heated for 3 hours. Additional aliquots of P(o-tol)₃ (13 mg, 0.19 mmol) Pd₂dba₃ (18 mg, 0.019 mmol) and triethylamine (80 uL) were added and the reaction mixture was heated at reflux for 24 hours. Additional aliquots of P(o-tol)₃ (13 mg, 0.19 mmol) Pd₂dba₃ (18 mg, 0.019 mmol) and triethylamine (80 uL, 0.57 mmol) were added and the reaction mixture was heated at reflux overnight. The reaction mixture was filtered through celite and the solvent evaporated. The residue was purified by preparative HPLC eluting with 10-20% acetonitrile in water with 0.1% formic acid. The fractions were combined and partially evaporated. The residue was taken up in DCM/aqueous sodium bicarbonate. After separation, the aqueous phase was extracted with DCM (×3). The extracts were combined, dried by passing through a hydrophobic frit and the solvent evaporated to give the product as a pale yellow solid (18 mg) (¹H NMR (300 MHz, CD₃OD) 8.05 (d, 1H, J=9 Hz), 7.9 (s, 1H), 7.3 (dd, 1H, J=1.6, 9 Hz), 7.1 (d, 1H, J=9 Hz), 6.45 (d, 1H, J=16 Hz), 6.15 (d, 1H, J=16 Hz), 6.1 (m, 1H), 4.4 (m, 1H), 3.65 (m, 1H), 3.4 (s, 3H), 2.8 (m, 2H), 2.65 (m, 2H), 2.0 (m, 2H), 1.7 (m, 2H), 1.7 (m, 1H), 1.6 (d, 3H, J=7 Hz), 1.2 (s, 3H), 1.1 (s, 3H), 1.0 (d, 3H, J=7 Hz), 0.8 (d, 3H, J=7 Hz); LC m/z[M+H]536).

Other Compounds in Table 2 were prepared by similar methods as described in the above Examples.

The LCMS data of the compounds are presented in Table 3

TABLE 3
LCMS retention time and [M + H].

| Compound | Formula | MS (m/z) | LC retention |
|---|---|---|---|
| number | MW | [M + H] | time (min) |
| 1 | C₂₇H₃₄N₆O₅ | 523 | 1.5 |
| | 522.59 | | |
| 2 | C₂₉H₃₈N₆O₅ | 551 | 1.64 |
| | 550.649 | | |
| 3 | C₂₉H₃₈N₆O₅ | 551 | 1.7 |
| | 50.649 | | |
| 4 | C₂₉H₃₈N₆O₅ | 552 | 2.17 |
| | 550.649 | | |
| 5 | C₂₉H₄₀N₆O₅ | 553 | 1.71 |
| | 551.637 | | |
| 6 | C₃₀H₄₀N₆O₅ | 565 | 1.59 |
| | 564.67 | | |
| 9 | C₃₅H₄₂N₆O₅ | 627 | 2.28 |
| | 626.74 | | |
| 11 | C₃₅H₄₀N₆O₇ | 657 | 1.49 |
| | 656.73 | | |
| 12 | C₃₆H₄₃N₇O₆ | 670 | 1.54 |
| | 669.77 | | |
| 13 | C₃₀H₄₀N₆O₆ | 581 | 1.65 |
| | 580.67 | | |
| 14 | C₃₀H₄₀N₆O₆ | 581 | 1.72 |
| | 580.67 | | |
| 16 | C₃₅H₄₂N₆O₅ | 627 | 1.87 |
| | 626.74 | | |
| 17 | C₃₅H₄₂N₆O₅ | 627 | 2.01 |
| | 626.74 | | |
| 18 | C₃₀H₃₈N₆O₇ | 595 | 1.48 |
| | 594.66 | | |
| 19 | C₃₁H₄₀N₆O₇ | 609 | 1.83 |
| | 608.68 | | |
| 20 | C₂₉H₄₁N₇O₃ | 536 | 0.89 |
| | 535.68 | | |
| 21 | C₃₁H₄₀N₆O₅ | 577 | 1.82 |
| | 576.68 | | |
| 22 | C₂₉H₃₉N₇O₄ | 550 | 1.41 |
| | 549.66 | | |
| 23 | C₃₀H₄₃N₇O₃ | 550 | 1.03 |
| | 549.71 | | |
| 24 | C₃₂H₄₂N₆O₄ | 575 | 2.09 |
| | 574.71 | | |
| 25 | C₃₀H₄₀N₆O₄ | 549 | 1.78 |
| | 548.67 | | |
| 26 | C₃₄H₄₀N₆O₅ | 613 | 1.99 |
| | 612.72 | | |
| 27 | C₃₄H₄₀N₆O₅ | 613 | 2.03 |
| | 612.72 | | |
| 28 | C₃₃H₄₅N₇O₅ | 620 | 1.04 |
| | 619.75 | | |
| 29 | C₃₄H₄₅N₇O₆ | 648 | 4.4 |
| | 647.76 | | |
| 30 | C₃₂H₄₃N₇O₅ | 606 | 1.09 |
| | 605.73 | | |
| 31 | C₃₂H₄₃N₇O₅ | 606 | 3.08 |
| | 605.73 | | |
| 33 | C₃₃H₄₅N₇O₅ | 619 | 4.0 |
| | 619.75 | | |
| 34 | C₃₂H₄₃N₇O₅ | 605 | 0.91 |
| | 605.73 | | |
| 35 | C₃₂H₄₄N₈O₄ | 605 | 0.97 |
| | 604.74 | | |
| 36 | C₃₃H₄₅N₇O₅ | 620 | 0.91 |
| | 619.75 | | |
| 37 | C₃₃H₄₅N₇O₅ | 620 | 3.08 |
| | 619.75 | | |
| 38 | C₃₅H₄₁N₇O₆ | 656 | 1.42 |
| | 655.74 | | |
| 39 | C₃₅H₄₁N₇O₆ | 656 | 1.32 |
| | 655.74 | | |
| 40 | C₃₄H₃₈N₆O₇ | 643 | 1.52 |
| | 642.70 | | |
| 42 | C₃₃H₃₈N₆O₆ | 615 | 1.36 |
| | 614.69 | | |
| 43 | C₃₂H₃₈N₈O₅ | 615 | 2.74 |
| | 614.69 | | |
| 44 | C₃₂H₄₃N₇O₅ | 606 | 1.05 |
| | 605.73 | | |
| 45 | C₃₆H₅₀N₈O₆ | 691 | 1.45 |
| | 690.83 | | |
| 46 | C₃₄H₄₁N₇O₇S | 692 | 1.5 |
| | 691.80 | | |
| 47 | C₃₇H₄₅N₇O₆ | 685 | 1.48 |
| | 683.79 | | |
| 48 | C₃₁H₄₂N₈O₅ | 607 | 0.93 |
| | 606.71 | | |
| 49 | C₃₇H₄₅N₇O₆ | 684 | 1.64 |
| | 683.79 | | |
| 50 | C₃₃H₃₉N₇O₅ | 614 | 1.19 |
| | 613.71 | | |
| 51 | C₃₂H₃₈N₈O₅ | 615 | 0.89 |
| | 614.69 | | |
| 52 | C₃₂H₃₈N₈O₅ | 615 | 0.93 |
| | 614.69 | | |
| 53 | C₂₇H₃₃ClN₆O₅ | 557, 559 | 1.49 |
| | 557.04 | | |
| 54 | C₂₉H₃₇N₇O₆ | 580 | 1.12 |
| | 579.65 | | |
| 55 | C₂₉H₃₉N₇O₅ | 566. | 0.89 |
| | 565.66 | | |
| 56 | C₃₂H₄₃N₇O₅ | 606 | 0.93 |
| | 605.73 | | |
| 57 | C₃₂H₄₃N₇O₅ | 606 | 0.91 |
| | 605.73 | | |
| 58 | C₃₂H₄₃N₇O₅ | 606 | 0.87 |
| | 605.73 | | |
| 59 | C₃₁H₄₀N₆O₆ | 593 | 1.41 |
| | 592.68 | | |
| 60 | C₃₄H₄₀N₆O₆ | 629 | 1.60 |
| | 628.72 | | |
| 61 | C₄₃H₅₃N₇O₅ | 626 | 1.41 |
| | 747.92 | | |
| 62 | C₃₁H₄₀N₆O₇ | 609 | 3.60 |
| | 608.68 | | |
| 63 | C₃₇H₄₅N₇O₆ | 684 | 3.93 |
| | 683.79 | | |
| 64 | C₃₈H₄₇N₇O₆ | 698 | 1.75 |
| | 697.82 | | |
| 65 | C₃₇H₄₆N₈O₆ | 699 | 1.18 |
| | 698.81 | | |
| 66 | C₃₉H₄₉N₇O₆ | 712 | 1.70 |
| | 711.85 | | |
| 67 | C₃₇H₅₁N₇O₇ | 706 | 1.51 |
| | 705.84 | | |
| 68 | C₃₆H₄₇N₉O₆ | 702 | 1.43 |
| | 701.81 | | |
| 69 | C₃₅H₄₈N₈O₆ | 677 | 1.04 |
| | 676.80 | | |
| 70 | C₃₆H₅₀N₈O₆ | 691 | 1.08 |
| | 690.83 | | |
| 71 | C₃₇H₅₂N₈O₆ | 705 | 2.69 |
| | 704.86 | | |
| 72 | C₃₆H₄₉N₇O₇ | 692 | 1.45 |
| | 691.82 | | |
| 73 | C₃₅H₄₉N₇O₅ | 648 | 3.72 |
| | 647.81 | | |
| 74 | C₃₃H₄₅N₇O₅ | 620 | 1.69 |
| | 619.75 | | |
| 75 | C₃₃H₄₅N₇O₅ | 620 | 1.59 |
| | 619.75 | | |
| 76 | C₃₅H₄₂N₆O₅ | 627.3 | 1.91 |
| | 626.74 | | |
| 77 | C₃₅H₄₂N₆O₅ | 627 | 2.14 |
| | 626.74 | | |
| 78 | C₃₄H₄₁N₇O₅ | 627 | 1.37 |
| | 627.73 | | |
| 79 | C₃₈H₄₉N₇O₅ | 684 | 1.38 |
| | 683.84 | | |
| 80 | C₃₁H₄₀N₆O₅ | 577 | 1.82 |
| | 576.68 | | |
| 81 | C₃₃H₃₇ClN₆O₅ | 633 | 1.79 |
| | 633.14 | | |
| 82 | C₃₀H₄₀N₆O₄ | 549 | 1.65 |
| | 548.67 | | |

¹H NMR data for selected Compounds are provided in Table 4.

TABLE 4
¹H NMR data.

| Compound | |
|---|---|
| number | ¹H NMR data (300 MHz) CD₃OD unless stated |
| 1 | (CDCl₃) 7.9 (m, 2H), 7.6 (m, 1H), 7.1 (m, 1H), 6.95 (d, 1H), 6.9 (m, 1H), 6.7 (m, 1H), 5.35 (m, |
| | 1H), 4.9 (m, 1H), 3.95 (m, 1H), 3.6-3.4 (m, 2H), 3.5 (s, 3H), 2.4 (m, 1H), 2.25 (m, 1H), 1.9 (m, |
| | 1H), 1.8-1.6 (m, 4H), 1.4 (m, 3H), 1.0 (m, 6H). |
| 3 | 8.05 (m, 1H), 7.7 (m, 2H), 7.3 (m, 1H), 7.1 (m, 1h), 6.6 (d, 1H), 6.35 (d, 1H), 5.75 (q, 1H), 4.95 |
| | (d, 1H), 4.5 (m, 1H), 3.6 (m, 1H), 3.4 (s, 3H), 2.85 (m, 1H), 2.3 (m, 1H), 2.0 (m, 2H), 1.7 (m, 2H), |
| | 1.6 (d, 3H), 1.55 (s, 3H), 1.4 (s, 3H), 0.95 (m, 6H). |
| 4 | 8.6 (s, 1H), 7.8 (d, 1H), 7.6 (m, 1H), 7.5 (m, 1H), 6.3 (m, 2H), 5.9 (q, 1H), 5.1 (d, 1H), 4.4 (m, |
| | 1H), 3.6 (m, 1H), 3.5 (s, 3H), 2.8 (m, 1H), 2.2 (m, 1H), 1.9-1.7 (m, 4H), 1.55 (d, 3H), 1.5 (s, 3H), |
| | 1.4 (s, 3H), 1.0 (m, 6H). |
| 15 | 8.05 (d, 1H), 7.6 (d, 1H), 7.45 (m, 2H), 7.1 (d, 1h), 6.4 (d, 1H), 6.35 (d, 1H), 6.0 (m, 1H), 5.2 (m, |
| | 1H), 4.4 (m, 2H), 4.0 (m, 1H), 3.6 (m, 1H), 3.4 (s, 3H), 3.0 (m, 1H), 2.8 (m, 2H), 2.5 (m, 1H), 2.2 |
| | (m, 2H), 1.9-1.6 (m, 7H), 1.55 (d, 3H), 1.1 (m, 6H), 1.0 (m, 6H). |
| 23 | 8.1 (m, 1H), 7.8 (m, 1H), 7.65 (m, 1H), 7.3 (m, 1H), 7.15 (m, 1H), 6.7 (m, 1H), 6.45 (m, 1H), 5.8, |
| | 5.1 (m, 1H), 4.3 (m, 1H), 3.65 (m, 1H), 3.45, 3.4 (2s, 3H), 3.0-2.8 (m, 2H), 2.75, 2.6 (2 s, 3H), |
| | 2.5 (m, 1H), 2.2 (m, 2H), 1.8 (m, H), 1.5, 1.0 (2d, 3H), 1.3, 1.2 (2s, 3H), 1.15 (s, 3H), 0.95, 0.8 |
| | (2m, 6H) (multiple conformers). |
| 44 | 8.05 (d, 1H), 7.65 (d, 1H), 7.5 (m, 1H), 7.4 (d, 1H), 7.1 (d, 1H), 6.35 (d, 1h), 6.3 (d, 1H), 5.95 (q, |
| | 1H), 5.65 (t, 1H), 4.45 (m, 1H), 3.7 (m, 2H), 3.4 (s, 3H), 2.8 (m, 4H), 2.6 (m, 4H), 2.2 (m, 2H), |
| | 1.9-1.2 (m, 9H) (multiple conformers). |
| 60 | 8.05 (d, 1H), 7.60 (d, 1H), 7.5 (m, 1H), 7.4 (d, 1H), 7.2 (d, 2H), 7.1 (d, 1H), 6.85 (d, 2H), 6.3 (d, |
| | 1h), 6.2 (d, 1H), 5.95 (q, 1H), 5.6 (m, 1H), 4.45 (m, 1H), 3.8 (s, 3H), 3.4 (s, 3H), 3.05 (m, 2H), |
| | 2.8 (m, 1H), 2.0 (m, 2H), 1.9-1.7 (m, 2H), 1.6, 1.3 (2d, 3H), 1.4 (s, 3H), 1.3 (s, 3H) (multiple |
| | conformers). |
| 61 | 8.5 (d, 1H), 7.9 (m, 6H), 7.6 (m, 4H), 7.45 (d, 1H), 7.3 (d, 1H), 7.0 (m, 1H), 6.8 (m, 1H), 4.5 (m, |
| | 1H), 3.8 (m, 1H), 3.6 (s, 3H), 3.5 (s, 2H), 3.2 (m, 2H), 2.5 (m, 2H), 2.4 (m, 2H), 2.1-1.5 (m, 9H), |
| | 1.7, 1.6 (2s, 3H), 1.4, 1.3 (2s, 3H), 0.9 (m, 3H), 0.75 (m, 3H) (multiple conformers). |
| 82 | 8.1 (d, 1H), 8.0 (m, 1H), 7.7 (m, 1H), 7.25 (d, 1H), 7.1 (d, 1H), 6.6 (d, 1H), 6.2 (d, 1H), 5.85 (m, |
| | 1H), 4.5 (m, 1H), 3.9 (m, 1H), 3.4 (s, 3H), 2.8 (m, 2H), 2.4 (m, 1H), 2.1 (m, 1H), 1.9-1.7 (m, 5H), |
| | 1.5 (s, 3H), 1.35 (m, 3H), 1.35 (m, 3H), 1.3 (m, 3H), 1.0 (d, 3H) (multiple conformers). |

### Results

### Functional Peptidyl Prolyl Isomerase (PPlase) Spectrophotometric Assay

Cyclophilins are enzymes that catalyse the cis-trans isomerisation of peptide bonds to the amino acid proline. This event is important in many biological processes, including protein folding and signal transduction. Cyclosporins and the compounds of the present invention inhibit this catalytic activity. The method to measure this activity and its inhibition is similar to that described by Jankowski (Analytical Biochemistry, 252, 2, 299-307 (1997)). The assay measures the cis to trans isomerisation of a peptide substrate catalysed by a PPlase enzyme using a UV/Vis spectrophotometer. This assay is a manual single cuvette-based assay. A first order rate equation is fitted to the absorbance data to obtain a rate constant. The catalytic rate is calculated from the enzymatic rate minus the background rate. The Ki (the concentration required to produce half maximum inhibition) for an inhibitor is obtained from the rate constant plotted against the inhibitor concentration. Typically, a 6-point dose response curve is obtained from duplicates and a Ki determined. Data are given in Table 5 as Ki [nM] for cyclophilins A and D, expressed in column 2 of Table 5 as Ki (cypA)/Ki (cypD).

### Thapsigargin Treated Cell Death Assay in Jurkat Cells: Protection Against MPTP Opening

This assay demonstrates that the test compound is able to protect from thapsigargin induced cell death in Jurkat cells (immortalised human T-lymphocyte cell line; Bortner et al (J. Biol. Chem., July, 30, 274, 31, 21953-21962 (1999)).

Thapsigargin raises intracellular free calcium (Ca²⁺) by potently inhibiting the endoplasmic reticulum Ca-ATPase, which sequesters Ca²⁺ from the cytosol. The increased cytosolic Ca²⁺ concentration is buffered by mitochondria by exchanging Ca²⁺ for K, leading to depolarisation in the inner mitochondrial membrane and permeability transition.

The thapsigargin treated Jurkat cell death assay is a homogeneous 96 well plate based assay for cell death, instigated by the opening of the mitochondrial permeability transition pore (MPTP). The addition of thapsigargin to Jurkat cells results in an influx of calcium, the increase in intracellular calcium will initially be buffered by the mitochondria, but eventually the increasing calcium concentration will result in the opening of the MPTP, resulting in cell death.

CellTox™ Green is a dye which is unable to enter the cell until cell death leads to the loss of membrane integrity. Once this happens the CellTox™ Green enters the cell, binds to the cell's DNA and emits a fluorescent signal. If compounds inhibit the opening of the pore in response to thapsigargin or inhibit the resulting cell death pathways, then the gain in fluorescence normally seen when thapsigargin is added is inhibited. Unless otherwise stated, the assay is typically run using a range of three compound concentrations of 1, 3 and 10 μM in triplicate and is read kinetically over a period of 24 hours. Compounds are added 30 minutes before the addition of thapsigargin. Compound only controls and positive control, cyclosporin A (CsA) (3 and 10 μM) are also included in the assay. Results are typically calculated at the 8 hour time point and the kinetic data is also reported.

This study demonstrates that the test compound is able to protect from thapsigargin induced cell death in SH-SY5Y cells (human neuroblastoma cell line). The assay is carried out as described for the Jurkat cells but using the SH-SY5Y (human neuroblastoma) cell line instead of Jurkat cells. The results are shown in the fourth column of Table 5 (SY5Y [10 μM]% recovery). Positive numbers indicate protection from cell death, negative numbers indicate compound toxicity.

### Calcium Retention Capacity (CRC) Assay in Permeabilised HepG2 Cells

This assay demonstrates that the test compound is able to directly inhibit opening of the MPTP in permeabilised cells. The use of permeabilised cells means that inhibitory compounds that may not normally enter non-permeabilised cells are more likely to be active.

The calcium retention capacity (CRC) is a measure of the capability of mitochondria to retain calcium, primarily in the form of calcium phosphates, in the mitochondrial matrix. By storing calcium in the form of osmotically inactive precipitates the mitochondria contribute to the buffering of cytosolic free calcium levels and thereby to the regulation of calcium-dependent cellular processes, in particular the opening of the mitochondrial permeability transition pore (MPTP).

In a CRC assay, the calcium indicator Calcium Green-5N (InVitrogen) is utilized. Calcium Green-5N is an extra-mitochondrial dye which shows an increase in fluorescence when in the presence of calcium. The assay is carried out in HepG2 (human hepatocellular carcinoma) cells which are digitonin permeabilized. Trains of calcium pulses (typically 5 μM additions) are added which results in a sudden increase in fluorescence which is then reduced by mitochondrial buffering. The cells will reach a point at which the calcium addition is no longer buffered, an indication that the MPTP has opened (mitochondrial permeability transition).

The experiment is performed with and without inhibitor. A compound that acts to inhibit the mitochondrial pore opening will result in a greater requirement for calcium to reach the transition point. The results are normalised to a positive control (cyclosporin A) in each experiment. The results are shown in column 3 of Table 5 as CRC [10 μM] (ratio vs CsA). The method of digitonin permeabilised whole cell CRC assay is similar to that used by Chiara et al. (PLoS One, March, 3, 3, e1852 (2008)).

TABLE 5
Results of biochemical and cellular assays.

| | | | CRC | |
|---|---|---|---|---|
| | | | [10 μM] | |
| | Compound | Ki [nM] | (ratio vs | SYSY [10 μM] % |
| Structure | number | CypA/D | CsA) | recovery |
| 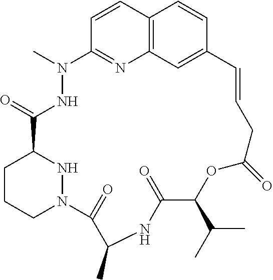 [SMILES: CC(C)[C@@H]1OC(=O)C/C=C/c2ccc3ccc(nc3c2)N(C)NC(=O)[C@@H]2CCCN(N2)C(=O)[C@H](C)NC1=O] | 1 | 25/54 | 35 (0.95) | 8.4 |
| 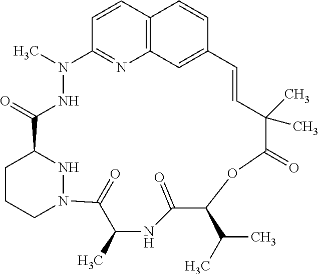 [SMILES: CC(C)[C@@H]1OC(=O)C(C)(C)/C=C/c2ccc3ccc(nc3c2)N(C)NC(=O)[C@@H]2CCCN(N2)C(=O)[C@H](C)NC1=O] | 2 | 5/17 | 26 (0.9875) | 40.4 |
| 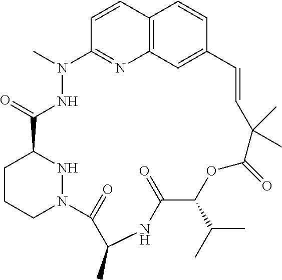 [SMILES: CC(C)[C@H]1OC(=O)C(C)(C)/C=C/c2ccc3ccc(nc3c2)N(C)NC(=O)[C@@H]2CCCN(N2)C(=O)[C@H](C)NC1=O] | 3 | 550/1300 | 10000 (0.5) | 0.6 |
| 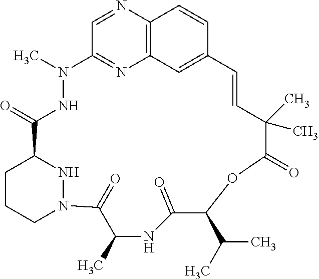 [SMILES: CC(C)[C@@H]1OC(=O)C(C)(C)/C=C/c2ccc3ncc(nc3c2)N(C)NC(=O)[C@@H]2CCCN(N2)C(=O)[C@H](C)NC1=O] | 4 | 3.3/13 | 16.4 | — |
| 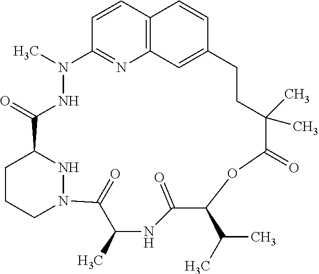 [SMILES: CC(C)[C@@H]1OC(=O)C(C)(C)CCc2ccc3ccc(nc3c2)N(C)NC(=O)[C@@H]2CCCN(N2)C(=O)[C@H](C)NC1=O] | 5 | >10 000 | — | — |
| 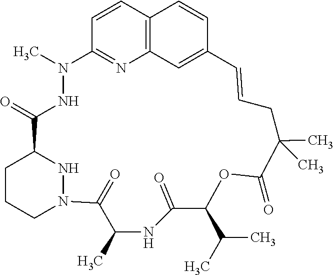 [SMILES: CC(C)[C@@H]1OC(=O)C(C)(C)C/C=C/c2ccc3ccc(nc3c2)N(C)NC(=O)[C@@H]2CCCN(N2)C(=O)[C@H](C)NC1=O] | 6 | >10 000 | — | — |
| 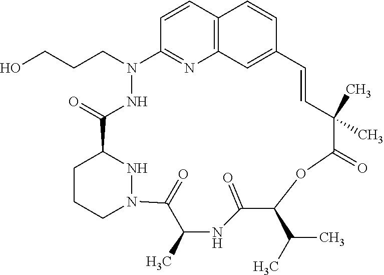 | 7 | 36.7/8.1 | — | — |
| 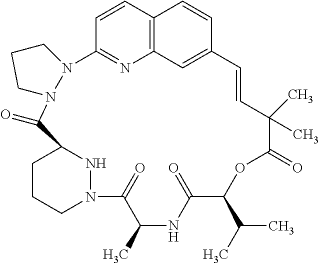 [SMILES: CC(C)[C@@H]1OC(=O)C(C)(C)/C=C/c2ccc3ccc(nc3c2)N2CCCN2C(=O)[C@@H]2CCCN(N2)C(=O)[C@H](C)NC1=O] | 8 | 13.9/15 | — | — |
| 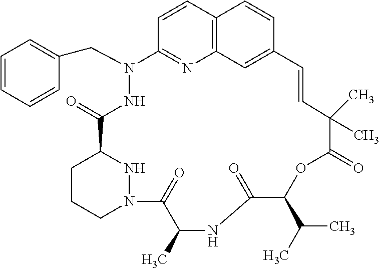 [SMILES: CC(C)[C@@H]1OC(=O)C(C)(C)/C=C/c2ccc3ccc(nc3c2)N(Cc2ccccc2)NC(=O)[C@@H]2CCCN(N2)C(=O)[C@H](C)NC1=O] | 9 | 1.7/4.3 | 279.4 (1) | 87.067 |
| 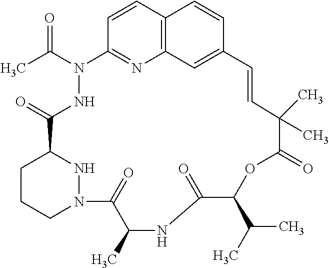 [SMILES: CC(=O)N1NC(=O)[C@@H]2CCCN(N2)C(=O)[C@H](C)NC(=O)[C@H](C(C)C)OC(=O)C(C)(C)/C=C/c2ccc3ccc1nc3c2] | 10 | 5.3/8.7 | — | — |
| 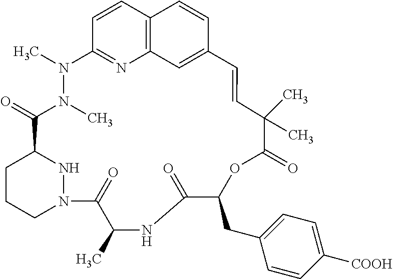 [SMILES: C[C@@H]1NC(=O)[C@H](Cc2ccc(C(=O)O)cc2)OC(=O)C(C)(C)/C=C/c2ccc3ccc(nc3c2)N(C)N(C)C(=O)[C@@H]2CCCN(N2)C1=O] | 11 | 280/83 | 20 (0.80) | −44 |
| 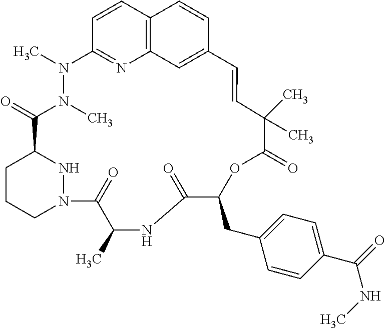 [SMILES: CNC(=O)c1ccc(C[C@@H]2OC(=O)C(C)(C)/C=C/c3ccc4ccc(nc4c3)N(C)N(C)C(=O)[C@@H]3CCCN(N3)C(=O)[C@H](C)NC2=O)cc1] | 12 | 240/49 | 15 (0.75) | 25 |
| 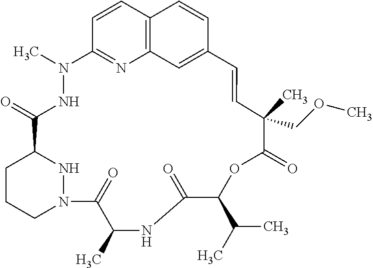 [SMILES: COC[C@@]1(C)/C=C/c2ccc3ccc(nc3c2)N(C)NC(=O)[C@@H]2CCCN(N2)C(=O)[C@H](C)NC(=O)[C@H](C(C)C)OC1=O] | 13 | 4.9/11 | 35 (0.95) | 8.4 |
| 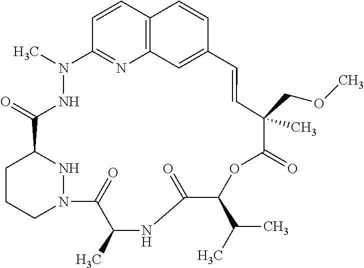 [SMILES: COC[C@]1(C)/C=C/c2ccc3ccc(nc3c2)N(C)NC(=O)[C@@H]2CCCN(N2)C(=O)[C@H](C)NC(=O)[C@H](C(C)C)OC1=O] | 14 | 17/25 | 40 (1.09) | 9.3 |
| 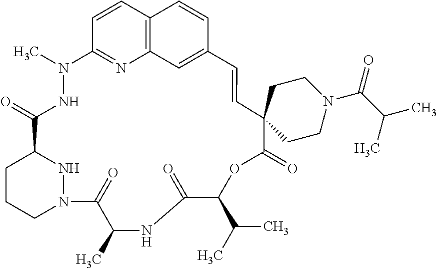 | 15 | 1.7/9.9 | 38 (1) | −1.05 |
| 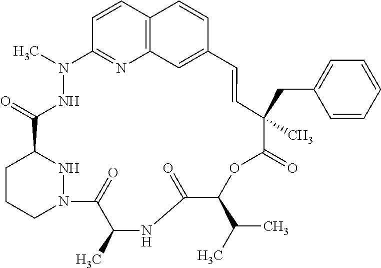 [SMILES: CC(C)[C@@H]1OC(=O)[C@](C)(Cc2ccccc2)/C=C/c2ccc3ccc(nc3c2)N(C)NC(=O)[C@@H]2CCCN(N2)C(=O)[C@H](C)NC1=O] | 16 | 1.7/14 | 25 (0.83) | 62 |
| 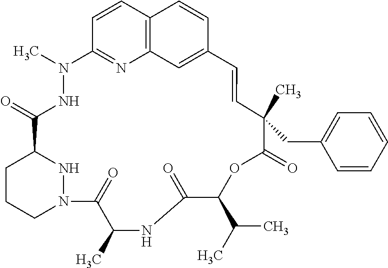 [SMILES: CC(C)[C@@H]1OC(=O)[C@@](C)(Cc2ccccc2)/C=C/c2ccc3ccc(nc3c2)N(C)NC(=O)[C@@H]2CCCN(N2)C(=O)[C@H](C)NC1=O] | 17 | 1.7/9 | 33.3 (0.95) | 87.8 |
| 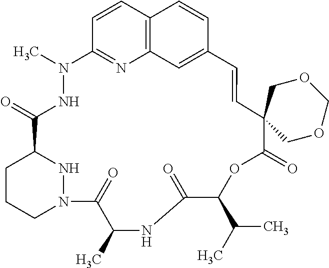 | 18 | 260/370 | 10 (0.33) | — |
| 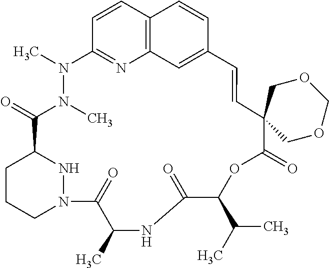 | 19 | 23/150 | 30 (0.77) | 5.7 |
| 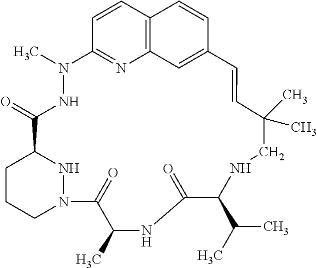 [SMILES: CC(C)[C@@H]1NCC(C)(C)/C=C/c2ccc3ccc(nc3c2)N(C)NC(=O)[C@@H]2CCCN(N2)C(=O)[C@H](C)NC1=O] | 20 | 100/140 | 22.5 (0.57) | — |
| 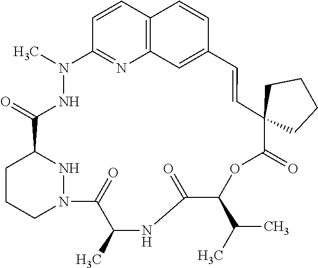 | 21 | 2.5/6.1 | 32.5 (0.93) | 33.8 |
| 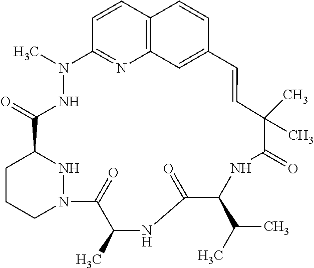 [SMILES: CC(C)[C@@H]1NC(=O)C(C)(C)/C=C/c2ccc3ccc(nc3c2)N(C)NC(=O)[C@@H]2CCCN(N2)C(=O)[C@H](C)NC1=O] | 22 | 13/27 | 25 (0.83) | 4 |
| 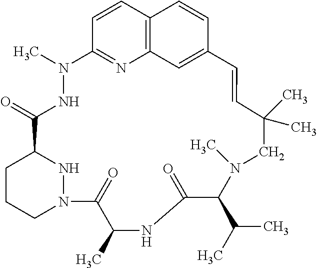 [SMILES: CC(C)[C@H]1C(=O)N[C@@H](C)C(=O)N2CCC[C@H](N2)C(=O)NN(C)c2ccc3ccc(cc3n2)/C=C/C(C)(C)CN1C] | 23 | 92/260 | 12.5 (0.38) | — |
| 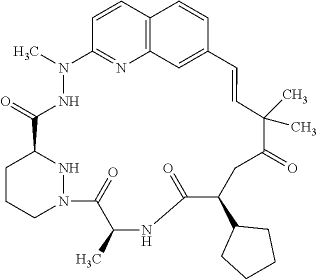 [SMILES: C[C@@H]1NC(=O)[C@H](C2CCCC2)CC(=O)C(C)(C)/C=C/c2ccc3ccc(nc3c2)N(C)NC(=O)[C@@H]2CCCN(N2)C1=O] | 24 | 19/12 | — | — |
| 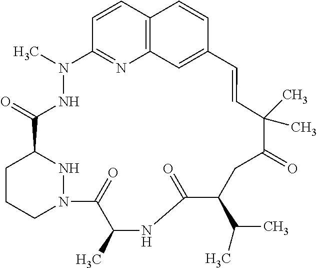 [SMILES: CC(C)[C@@H]1CC(=O)C(C)(C)/C=C/c2ccc3ccc(nc3c2)N(C)NC(=O)[C@@H]2CCCN(N2)C(=O)[C@H](C)NC1=O] | 25 | 17.5/6.2 | 20 (0.8) | 26.2 |
| 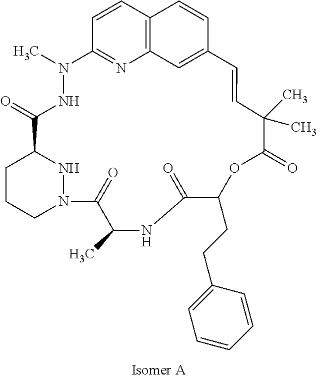 [SMILES: C[C@@H]1NC(=O)C(CCc2ccccc2)OC(=O)C(C)(C)/C=C/c2ccc3ccc(nc3c2)N(C)NC(=O)[C@@H]2CCCN(N2)C1=O] | 26 | 6700/1300 | — | — |
| 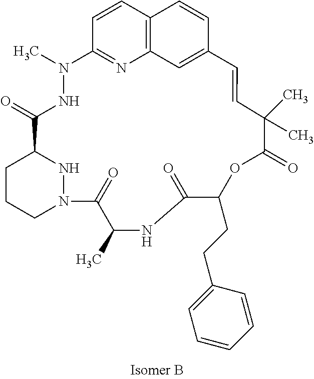 [SMILES: C[C@@H]1NC(=O)C(CCc2ccccc2)OC(=O)C(C)(C)/C=C/c2ccc3ccc(nc3c2)N(C)NC(=O)[C@@H]2CCCN(N2)C1=O] | 27 | 21/23 | 36.67 (0.81) | — |
| 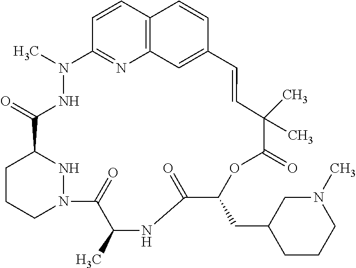 [SMILES: C[C@@H]1NC(=O)[C@@H](CC2CCCN(C)C2)OC(=O)C(C)(C)/C=C/c2ccc3ccc(nc3c2)N(C)NC(=O)[C@@H]2CCCN(N2)C1=O] | 28 | 5900/— | 5 (0.17) | — |
| 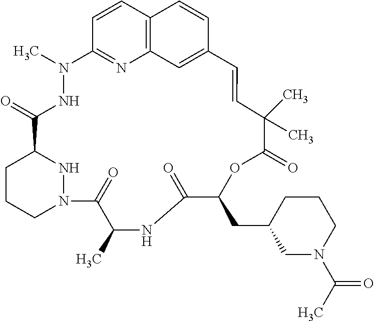 [SMILES: CC(=O)N1CCC[C@@H](C[C@@H]2OC(=O)C(C)(C)/C=C/c3ccc4ccc(nc4c3)N(C)NC(=O)[C@@H]3CCCN(N3)C(=O)[C@H](C)NC2=O)C1] | 29 | 51/170 | 20 (0.67) | −12 |
| 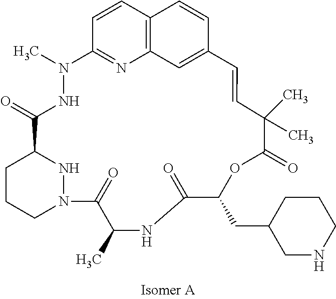 [SMILES: C[C@@H]1NC(=O)[C@@H](CC2CCCNC2)OC(=O)C(C)(C)/C=C/c2ccc3ccc(nc3c2)N(C)NC(=O)[C@@H]2CCCN(N2)C1=O] | 30 | —/450 | 10 (0.33) | 10 |
| 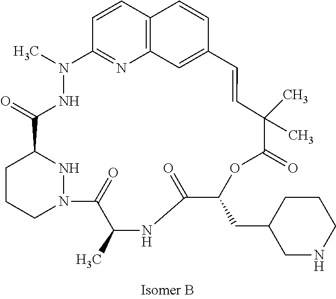 [SMILES: C[C@@H]1NC(=O)[C@@H](CC2CCCNC2)OC(=O)C(C)(C)/C=C/c2ccc3ccc(nc3c2)N(C)NC(=O)[C@@H]2CCCN(N2)C1=O] | 31 | 790/180 | 10 (0.33) | 12 |
| 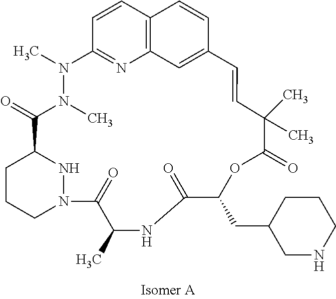 [SMILES: C[C@@H]1NC(=O)[C@@H](CC2CCCNC2)OC(=O)C(C)(C)/C=C/c2ccc3ccc(nc3c2)N(C)N(C)C(=O)[C@@H]2CCCN(N2)C1=O] | 32 | 2200/4900 | 5 (0.20) | −3 |
| 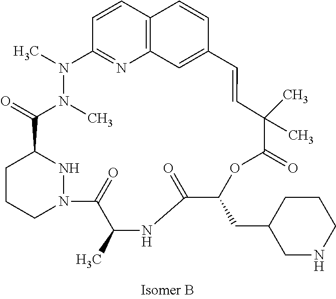 [SMILES: C[C@@H]1NC(=O)[C@@H](CC2CCCNC2)OC(=O)C(C)(C)/C=C/c2ccc3ccc(nc3c2)N(C)N(C)C(=O)[C@@H]2CCCN(N2)C1=O] | 33 | 280/3800 | 5 (20) | 9 |
| 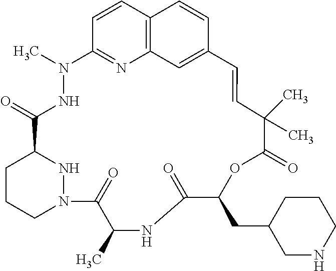 [SMILES: C[C@@H]1NC(=O)[C@H](CC2CCCNC2)OC(=O)C(C)(C)/C=C/c2ccc3ccc(nc3c2)N(C)NC(=O)[C@@H]2CCCN(N2)C1=O] | 34 | 790/1700 | 8.3 (0.33) | 9 |
| 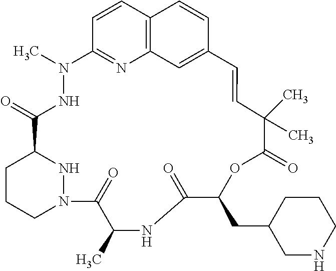 [SMILES: C[C@@H]1NC(=O)[C@H](CC2CCCNC2)OC(=O)C(C)(C)/C=C/c2ccc3ccc(nc3c2)N(C)NC(=O)[C@@H]2CCCN(N2)C1=O] | 35 | 1100/1100 | 10 (0.41 | 37 |
| 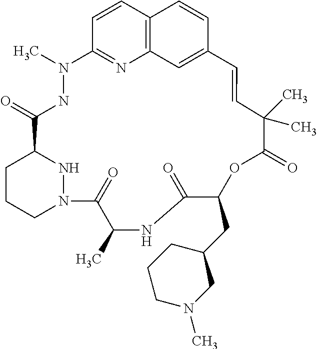 [SMILES: C[C@@H]1NC(=O)[C@H](C[C@@H]2CCCN(C)C2)OC(=O)C(C)(C)/C=C/c2ccc3ccc(nc3c2)N(C)NC(=O)[C@@H]2CCCN(N2)C1=O] | 36 | 390/1400 | 7.5 (0.42) | 19 |
| 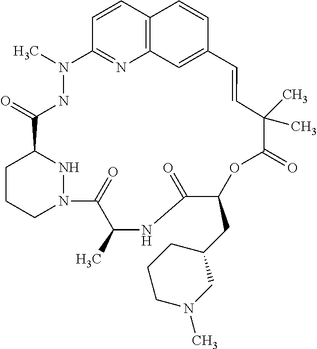 [SMILES: C[C@@H]1NC(=O)[C@H](C[C@H]2CCCN(C)C2)OC(=O)C(C)(C)/C=C/c2ccc3ccc(nc3c2)N(C)NC(=O)[C@@H]2CCCN(N2)C1=O] | 37 | 150/160 | 5 (0.33) | 41 |
| 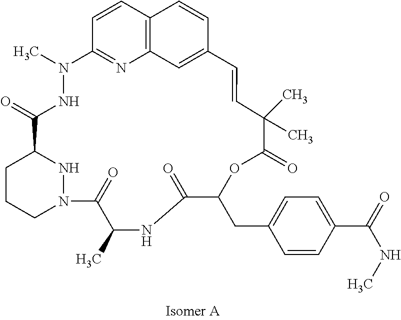 [SMILES: CNC(=O)c1ccc(CC2OC(=O)C(C)(C)/C=C/c3ccc4ccc(nc4c3)N(C)NC(=O)[C@@H]3CCCN(N3)C(=O)[C@H](C)NC2=O)cc1] | 38 | 160/190 | 7.5 (0.33) | 8 |
| 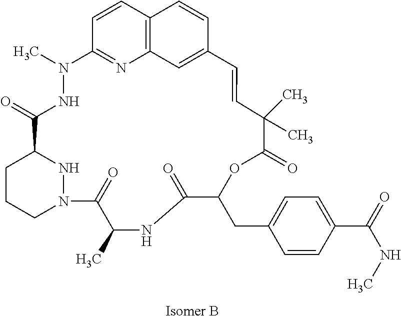 [SMILES: CNC(=O)c1ccc(CC2OC(=O)C(C)(C)/C=C/c3ccc4ccc(nc4c3)N(C)NC(=O)[C@@H]3CCCN(N3)C(=O)[C@H](C)NC2=O)cc1] | 39 | 30/36 | 15 (0.67) | 0 |
| 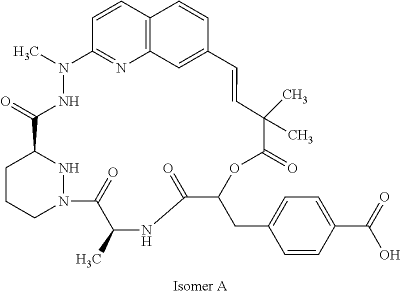 [SMILES: C[C@@H]1NC(=O)C(Cc2ccc(C(=O)O)cc2)OC(=O)C(C)(C)/C=C/c2ccc3ccc(nc3c2)N(C)NC(=O)[C@@H]2CCCN(N2)C1=O] | 40 | — | 5 (0.33) | 51 |
| 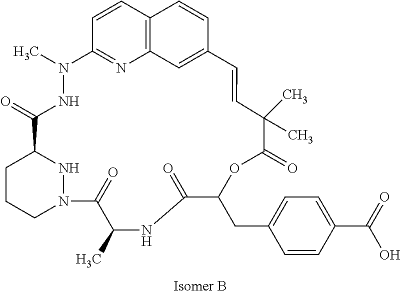 [SMILES: C[C@@H]1NC(=O)C(Cc2ccc(C(=O)O)cc2)OC(=O)C(C)(C)/C=C/c2ccc3ccc(nc3c2)N(C)NC(=O)[C@@H]2CCCN(N2)C1=O] | 41 | 30/220 | 10 (0.67) | 0 |
| 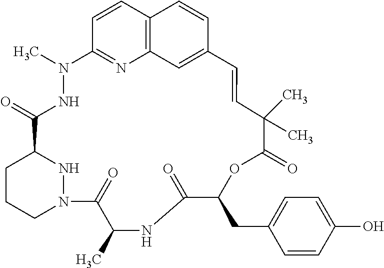 [SMILES: C[C@@H]1NC(=O)[C@H](Cc2ccc(O)cc2)OC(=O)C(C)(C)/C=C/c2ccc3ccc(nc3c2)N(C)NC(=O)[C@@H]2CCCN(N2)C1=O] | 42 | 1.2/5.8 | 25 (0.8) | −8 |
| 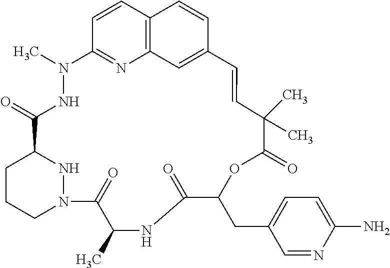 [SMILES: C[C@@H]1NC(=O)C(Cc2ccc(N)nc2)OC(=O)C(C)(C)/C=C/c2ccc3ccc(nc3c2)N(C)NC(=O)[C@@H]2CCCN(N2)C1=O] | 43 | 44/40 | 10 (0.67) | 10 |
| 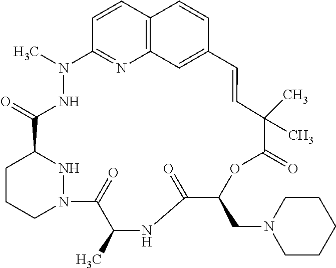 [SMILES: C[C@@H]1NC(=O)[C@H](CN2CCCCC2)OC(=O)C(C)(C)/C=C/c2ccc3ccc(nc3c2)N(C)NC(=O)[C@@H]2CCCN(N2)C1=O] | 44 | 590/140 | 5 (0.33) | −17 |
| 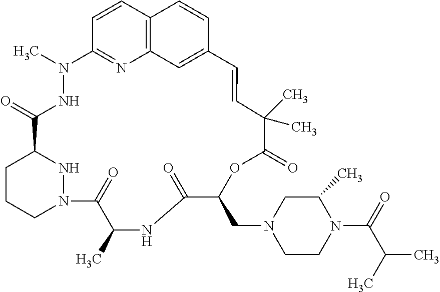 [SMILES: CC(C)C(=O)N1CCN(C[C@@H]2OC(=O)C(C)(C)/C=C/c3ccc4ccc(nc4c3)N(C)NC(=O)[C@@H]3CCCN(N3)C(=O)[C@H](C)NC2=O)C[C@@H]1C] | 45 | 61/140 | 15 (0.60) | −44 |
| 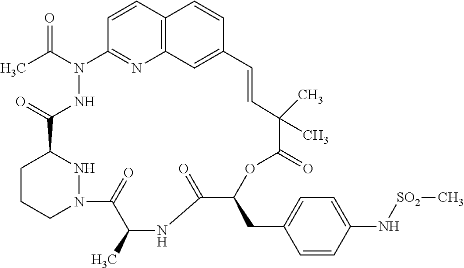 | 46 | 3/2.5 | 25 (1.00) | 29 |
| 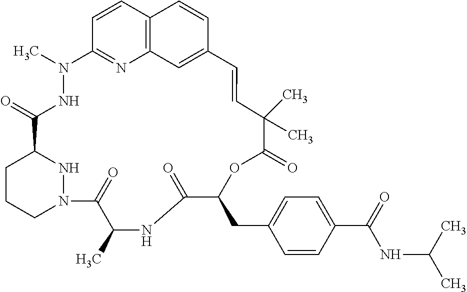 [SMILES: CC(C)NC(=O)c1ccc(C[C@@H]2OC(=O)C(C)(C)/C=C/c3ccc4ccc(nc4c3)N(C)NC(=O)[C@@H]3CCCN(N3)C(=O)[C@H](C)NC2=O)cc1] | 47 | 27/21 | 17.5 (1.00) | −6 |
| 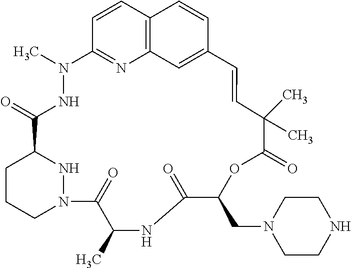 [SMILES: C[C@@H]1NC(=O)[C@H](CN2CCNCC2)OC(=O)C(C)(C)/C=C/c2ccc3ccc(nc3c2)N(C)NC(=O)[C@@H]2CCCN(N2)C1=O] | 48 | 26/11 | 10 (0.5) | 46 |
| 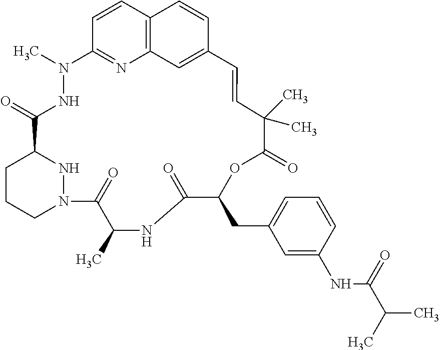 [SMILES: CC(C)C(=O)Nc1cccc(C[C@@H]2OC(=O)C(C)(C)/C=C/c3ccc4ccc(nc4c3)N(C)NC(=O)[C@@H]3CCCN(N3)C(=O)[C@H](C)NC2=O)c1] | 49 | 22/14 | 20 (1.00) | 40 |
| 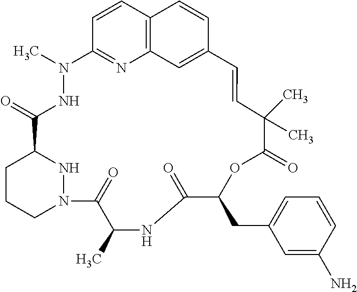 [SMILES: C[C@@H]1NC(=O)[C@H](Cc2cccc(N)c2)OC(=O)C(C)(C)/C=C/c2ccc3ccc(nc3c2)N(C)NC(=O)[C@@H]2CCCN(N2)C1=O] | 50 | 2.4/1.6 | 20 (1.00) | 25 |
| 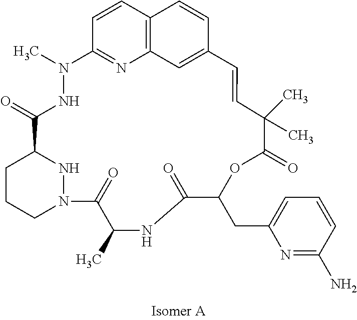 [SMILES: C[C@@H]1NC(=O)C(Cc2cccc(N)n2)OC(=O)C(C)(C)/C=C/c2ccc3ccc(nc3c2)N(C)NC(=O)[C@@H]2CCCN(N2)C1=O] | 51 | 5.4/9.6 | 22.5 (0.87) | 37 |
| 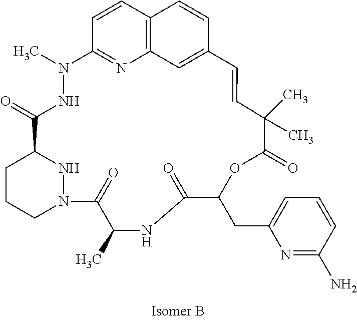 [SMILES: C[C@@H]1NC(=O)C(Cc2cccc(N)n2)OC(=O)C(C)(C)/C=C/c2ccc3ccc(nc3c2)N(C)NC(=O)[C@@H]2CCCN(N2)C1=O] | 52 | 290/420 | 10 (0.5) | 35 |
| 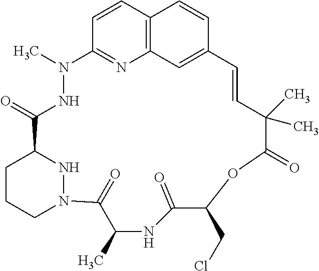 [SMILES: C[C@@H]1NC(=O)[C@H](CCl)OC(=O)C(C)(C)/C=C/c2ccc3ccc(nc3c2)N(C)NC(=O)[C@@H]2CCCN(N2)C1=O] | 53 | 15/28 | 25 (0.83) | 21 |
| 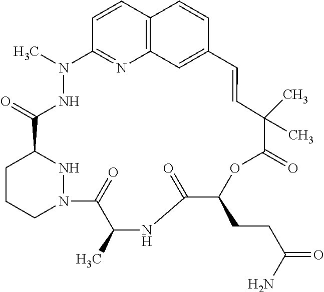 [SMILES: C[C@@H]1NC(=O)[C@H](CCC(N)=O)OC(=O)C(C)(C)/C=C/c2ccc3ccc(nc3c2)N(C)NC(=O)[C@@H]2CCCN(N2)C1=O] | 54 | 36/700 | 15 (0.50) | 10 |
| 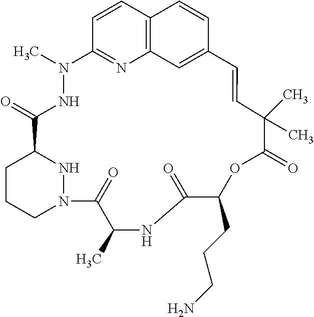 [SMILES: C[C@@H]1NC(=O)[C@H](CCCN)OC(=O)C(C)(C)/C=C/c2ccc3ccc(nc3c2)N(C)NC(=O)[C@@H]2CCCN(N2)C1=O] | 55 | 110/1900 | 5 (0.25) | 37 |
| 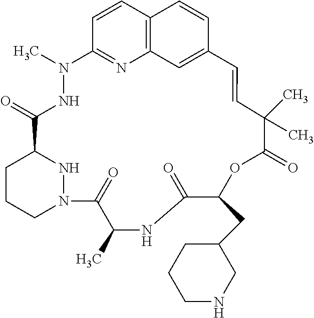 [SMILES: C[C@@H]1NC(=O)[C@H](CC2CCCNC2)OC(=O)C(C)(C)/C=C/c2ccc3ccc(nc3c2)N(C)NC(=O)[C@@H]2CCCN(N2)C1=O] | 56 | 295/56.6 | 8 (0.36) | 15 |
| 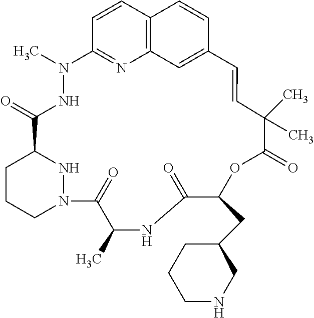 [SMILES: C[C@@H]1NC(=O)[C@H](C[C@@H]2CCCNC2)OC(=O)C(C)(C)/C=C/c2ccc3ccc(nc3c2)N(C)NC(=O)[C@@H]2CCCN(N2)C1=O] | 57 | 7.8/7.6 | 15 (0.75) | 48.48 |
| 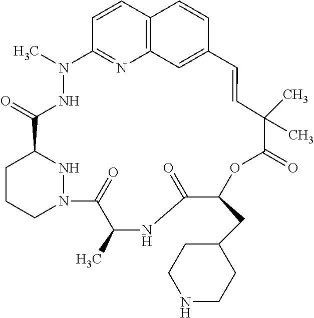 [SMILES: C[C@@H]1NC(=O)[C@H](CC2CCNCC2)OC(=O)C(C)(C)/C=C/c2ccc3ccc(nc3c2)N(C)NC(=O)[C@@H]2CCCN(N2)C1=O] | 58 | 80/54.5 | 8.3 (0.38) | −16 |
| 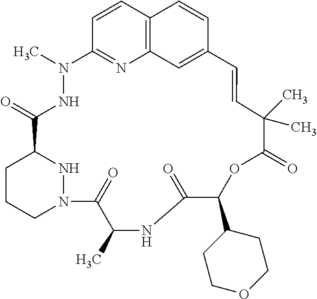 [SMILES: C[C@@H]1NC(=O)[C@H](C2CCOCC2)OC(=O)C(C)(C)/C=C/c2ccc3ccc(nc3c2)N(C)NC(=O)[C@@H]2CCCN(N2)C1=O] | 59 | 37/38 | 17.5 (0.85) | −4 |
| 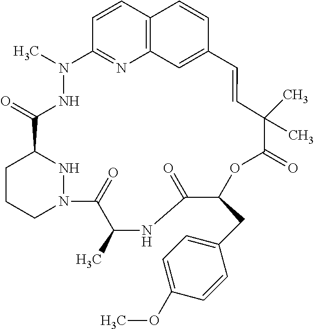 [SMILES: COc1ccc(C[C@@H]2OC(=O)C(C)(C)/C=C/c3ccc4ccc(nc4c3)N(C)NC(=O)[C@@H]3CCCN(N3)C(=O)[C@H](C)NC2=O)cc1] | 60 | 1.3/9.5 | 25 (0.80) | 37 |
| 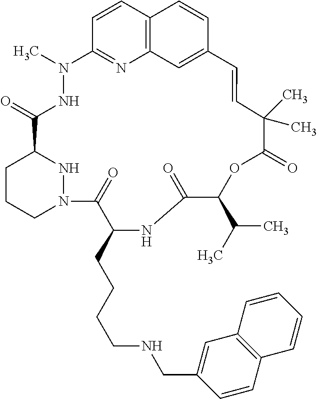 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCCCNCc2ccc3ccccc3c2)NC1=O] | 61 | 1.85/4.25 | 20 (0.78) | 35 |
| 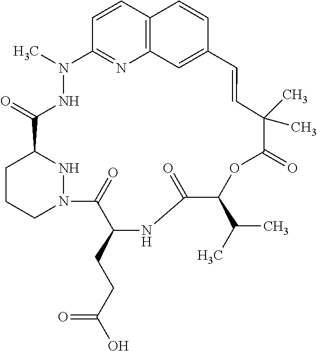 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)O)NC1=O] | 62 | 5.6/9.6 | — | — |
| 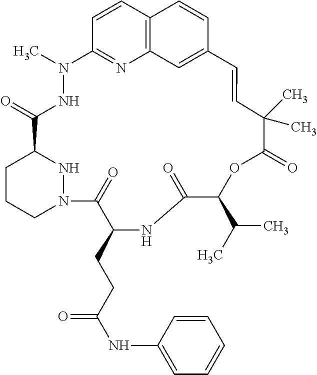 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)Nc2ccccc2)NC1=O] | 63 | 11/15 | 30 (1.00) | 48 |
| 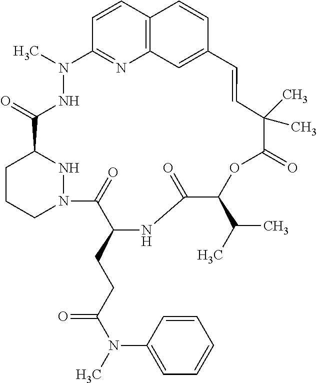 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)N(C)c2ccccc2)NC1=O] | 64 | 5.1/59 | 25 (1.00) | 44 |
| 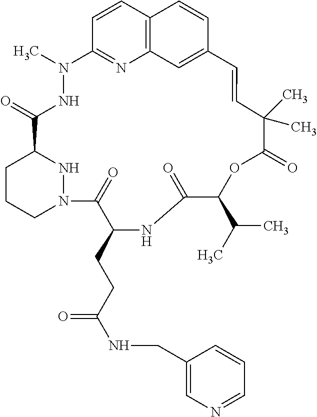 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)NCc2cccnc2)NC1=O] | 65 | 14/22 | 20 (0.90) | 51 |
| 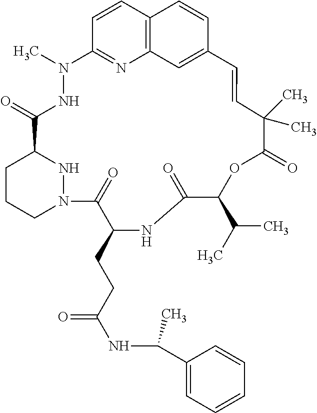 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)N[C@H](C)c2ccccc2)NC1=O] | 66 | 3.6/6.6 | 25 (0.80) | 63 |
| 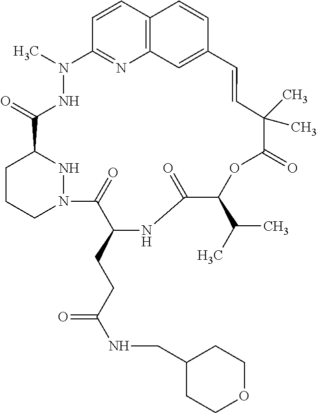 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)NCC2CCOCC2)NC1=O] | 67 | 40/26 | 25 (1.00) | 52 |
| 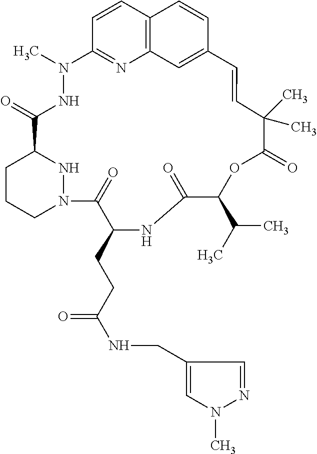 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)NCc2cnn(C)c2)NC1=O] | 68 | 16/9.9 | 25 (0.83) | 37 |
| 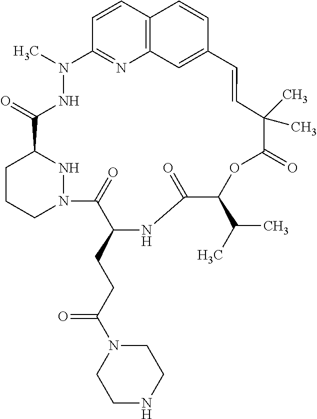 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)N2CCNCC2)NC1=O] | 69 | 34/41 | 20 (0.80) | 17 |
| 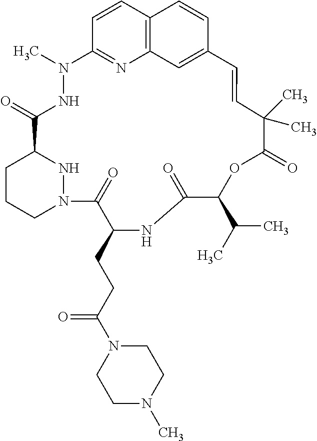 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)N2CCN(C)CC2)NC1=O] | 70 | 6.7/53 | 25 (1.00) | 44 |
| 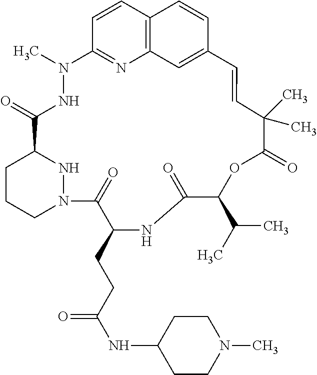 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)NC2CCN(C)CC2)NC1=O] | 71 | 51/16 | 18.3 (0.76) | 4 |
| 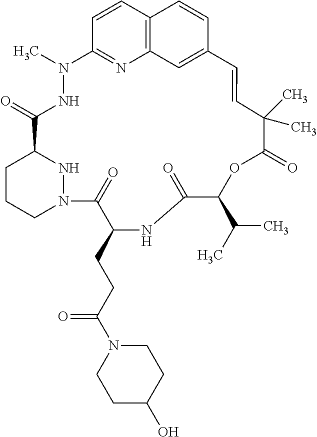 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)N2CCC(O)CC2)NC1=O] | 72 | 14/21 | 20 (0.80) | 43 |
| 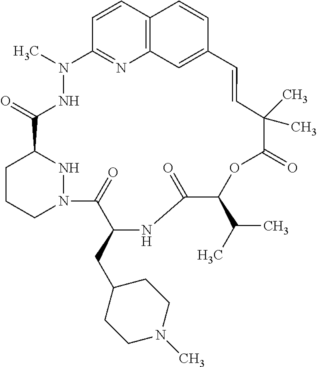 [SMILES: CC(C)[C@@H]1OC(=O)C(C)(C)/C=C/c2ccc3ccc(nc3c2)N(C)NC(=O)[C@@H]2CCCN(N2)C(=O)[C@H](CC2CCN(C)CC2)NC1=O] | 73 | 11/42 | 15 (0.75) | 43 |
| 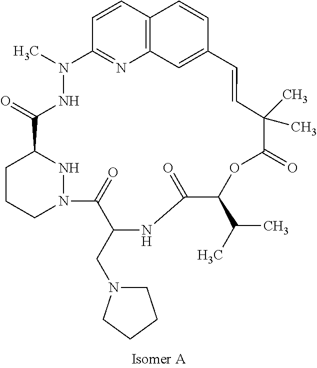 [SMILES: CC(C)[C@@H]1OC(=O)C(C)(C)/C=C/c2ccc3ccc(nc3c2)N(C)NC(=O)[C@@H]2CCCN(N2)C(=O)C(CN2CCCC2)NC1=O] | 74 | 5.3/8.2 | 20 (1.00) | — |
| 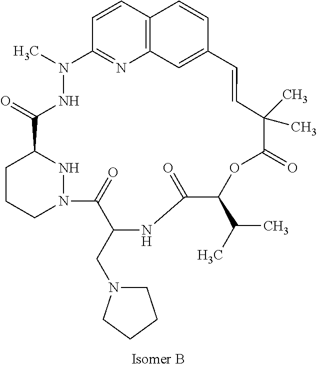 [SMILES: CC(C)[C@@H]1OC(=O)C(C)(C)/C=C/c2ccc3ccc(nc3c2)N(C)NC(=O)[C@@H]2CCCN(N2)C(=O)C(CN2CCCC2)NC1=O] | 75 | 390/2600 | — | — |
| 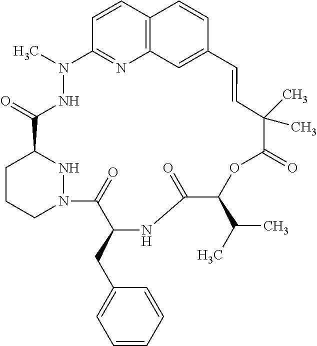 [SMILES: CC(C)[C@@H]1OC(=O)C(C)(C)/C=C/c2ccc3ccc(nc3c2)N(C)NC(=O)[C@@H]2CCCN(N2)C(=O)[C@H](Cc2ccccc2)NC1=O] | 76 | 5.6/6.3 | 33.9 (0.97) | 27.6 |
| 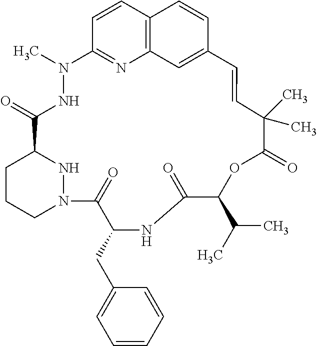 [SMILES: CC(C)[C@@H]1OC(=O)C(C)(C)/C=C/c2ccc3ccc(nc3c2)N(C)NC(=O)[C@@H]2CCCN(N2)C(=O)[C@@H](Cc2ccccc2)NC1=O] | 77 | —/700 | 15 (0.39) | — |
| 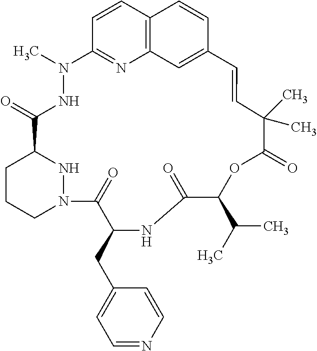 [SMILES: CC(C)[C@@H]1OC(=O)C(C)(C)/C=C/c2ccc3ccc(nc3c2)N(C)NC(=O)[C@@H]2CCCN(N2)C(=O)[C@H](Cc2ccncc2)NC1=O] | 78 | —/360 | >10,000 | — |
| 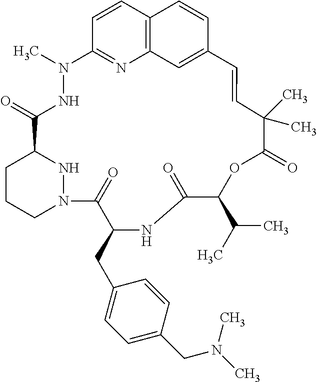 [SMILES: CC(C)[C@@H]1OC(=O)C(C)(C)/C=C/c2ccc3ccc(nc3c2)N(C)NC(=O)[C@@H]2CCCN(N2)C(=O)[C@H](Cc2ccc(CN(C)C)cc2)NC1=O] | 79 | 8.9/28 | 28.33 (0.85) | 1.8 |
| 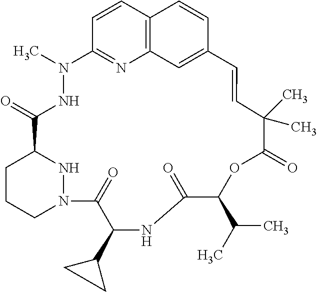 [SMILES: CC(C)[C@@H]1OC(=O)C(C)(C)/C=C/c2ccc3ccc(nc3c2)N(C)NC(=O)[C@@H]2CCCN(N2)C(=O)[C@H](C2CC2)NC1=O] | 80 | 4/22 | 25 (1.00) | 29.2 |
| 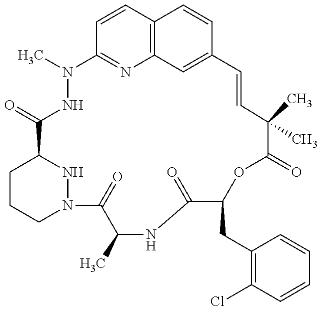 | 81 | 9.7/9.4 | 20 (0.67) | 44 |
| 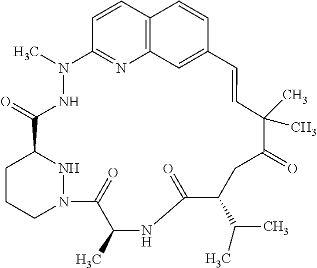 [SMILES: CC(C)[C@H]1CC(=O)C(C)(C)/C=C/c2ccc3ccc(nc3c2)N(C)NC(=O)[C@@H]2CCCN(N2)C(=O)[C@H](C)NC1=O] | 82 | 320/150 | — | — |

### Activity Against Acute Pancreatitis

Acute pancreatitis is mostly caused either by gallstones or by excessive alcohol consumption. Both causes converge on MPTP opening as a common mechanism to cause the disease (Mukherjee et al., Gut Online, 12 Jun. 2015). Blocking of the bile duct by gallstones prevents the outflow of bile acids, which are acutely toxic for the pancreatic acinar cells, causing excessive Ca²⁺ accumulation, MPTP opening and necrosis. Alcohol is partly transformed in the body into fatty acid ethyl esters which are also acutely toxic to the pancreatic acinar cells resulting in a Ca²⁺ spike, MPTP opening and necrosis. Inhibition of MPTP opening by cyclophilin inhibitors, including Compound (2), has been shown to be protective in animal models of acute pancreatitis (Mukherjee et al., Gut Online, 12 Jun. 2015).

### Protection of Cells Against Other Toxins

The toxicity mechanism described above for bile acid on pancreatic acinar cells is very similar to that of many other toxins, such as of aminoglycoside antibiotics on renal tubular cells, or on hair cells of the inner ear (Dehne et al., Hear Res., July, 169, 1-2, 47-55 (2002)), or of muscle protein on renal tubular cells (rhabdomyolysis; Chang et al. (The Journal of Nutritional Biochemistry, December, 110, 109134 (2022))).

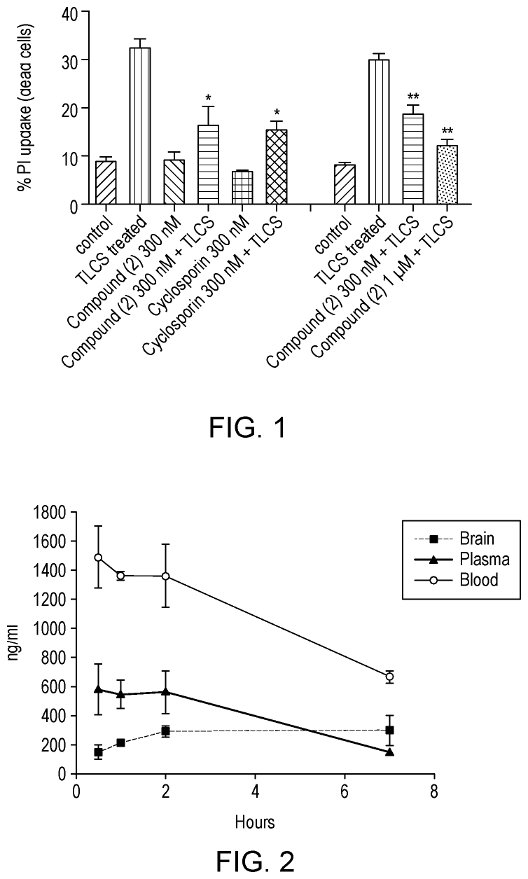

WO 2021/115299 (Zuo) discloses compounds that protect against acute kidney injury caused by many different causes. The same compounds are disclosed in U.S. Pat. No. 6,583,265 (C-Chem AG) as cyclophilin inhibitors. There is no enzyme inhibiting, receptor binding or ion channel activity known for these compounds other than inhibition of cyclophilins. It can therefore be concluded that the compounds of the present invention, which are potent cyclophilin inhibitors but have no activity on other enzymes or receptors, have the same spectrum of properties. This conclusion is confirmed by the activity of Compound (2) to protect pancreatic acinar cells against the toxicity of the bile acid taurolithocholic acid-3-sulphate (TLCS) illustrated in FIG. 1 which measures the level of propidium iodide (PI) as a measure of the number of dead pancreatic acinar cells (through necrosis) against a number of treatments with Compound (2) and cyclosporin A and controls.

FIG. 1 illustrates two sets of results. The single asterisk which appears in the left-hand set of results in FIG. 1 indicates that the result is significant over the control ‘TLCS treated’. The double asterisk which appears in the right-hand set of results in FIG. 1 indicates that the result is very significant over the control ‘TLCS treated’.

### Pharmacokinetics: Blood-Brain Barrier Penetration

Test compounds were administered to rodents (mice or rats) either orally, subcutaneously or intravenously. To determine blood or plasma concentrations blood samples were taken at different time points and analysed for the concentration of test compound by LC/MS. Brain concentrations were measured by sacrificing animals and LC/MS analysis of brain homogenate.

The results for Compounds (2), (19), (6) and (27) are summarised in Table 6 showing concentrations of the Compounds in blood plasma and the brain. All four Compounds showed that the Compounds passed the blood-brain barrier.

TABLE 6
Brain penetration results for selected Compounds of the invention.

| | Dose | Plasma | Brain |
|---|---|---|---|
| Compound | [mg/kg | concentration | concentration |
| number | intravenous] | [ng/mL at 30 min.] | [ng/mL at 30 min.] |
| 2 | 3 | 222 | 170 |
| 19 | 3 | 74 | 116 |
| 6 | 3 | 1216 | 197 |
| 27 | 3 | 1382 | 285 |

FIG. 2 illustrates graphs for the concentration (ng/ml) Compound (2) in blood, blood plasma and in the brain at 30 minutes to 7 hours after oral treatment. The calculated pharmacokinetic data is presented in Table 7.

TABLE 7
Pharmacokinetics of compound (2) in mice (strain
FVB/N) with oral dose of 10 mg/kg.

| PK parameter | Units | Plasma | Whole blood | Brain |
|---|---|---|---|---|
| T_{1/2} | hr | 3.0 | 5.45 | — |
| Cₘₐₓ | ng/mL | 582 | 1490 | 297 |
| Tₘₐₓ | hr | 0.5 | 0.5 | 7 |
| AUC₀₋ₜ | ng/mL/hr | 3180 | 12500 | — |
| Blood/plasma | Ratio | 2.87 | 2.87 | — |
| Blood/brain | Ratio | 0.72 | — | 0.72 |

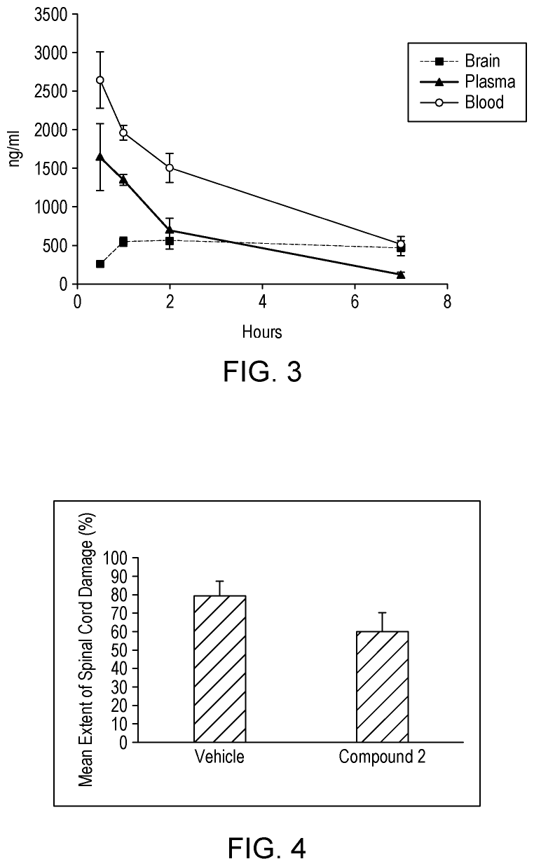

FIG. 3 illustrates graphs for the concentration (ng/ml) Compound (2) in blood, blood plasma and in the brain at 30 minutes to 7 hours after the same dosage administered sub-cutaneously. The calculated pharmacokinetic data is presented in Table 8.

TABLE 8
Pharmacokinetics of compound (2) in mice (strain
FVB/N) with 10 mg/kg subcutaneous administration.

| PK parameter | Units | Plasma | Whole blood | Brain |
|---|---|---|---|---|
| T_{1/2} | hr | 1.77 3.17 | 1.77 3.17 | |
| Cₘₐₓ | ng/mL | 1640 | 2640 | 563 |
| Tₘₐₓ | hr | 0.5 | 0.5 | 2.0 |
| AUC₀₋ₜ | ng/mL/hr | 3730 | 8150 | 3370 |
| Blood/plasma | Ratio | 2.2 | 2.2 | |
| Blood/brain | Ratio | 0.9 | | 0.9 |

### Experimental Allergic Encephalitis: Mouse Model of Multiple Sclerosis

Multiple sclerosis (MS) is a chronic inflammatory neurodegenerative disease of the central nervous system (CNS). Experimental autoimmune encephalomyelitis (EAE) is the most common animal model of human MS and especially useful to investigate neuroinflammatory pathways (Martin R, “Immunological aspects of experimental allergic encephalomyelitis and multiple sclerosis and their application for new therapeutic strategies’ in Mizuno, Y., Youdim, M. B. H., Calne, D. B., Horowski, R., Poewe, W., Riederer, P. (eds) (Advances in Research on Neurodegeneration, Springer, Vienna, https://doi.org/10.1007/978-3-70916844-8 6 (1997))).

C57BL/6 mice are the most commonly used strain. For the induction of EAE, typically, the immunogenic epitope MOG₃₅₋₅₅ of myelin oligodendrocyte glycoprotein is suspended in complete Freund's adjuvant (CFA) prior to immunization. Thereafter, pertussis toxin (PT) is applied on the day of immunization and a couple of days later to facilitate the induction of EAE. Animals are assessed by body weight change, EAE scoring and histopathological examination for both inflammation and demyelination.

Compound (2) was dissolved at a dose level of 75 mg/kg with a final concentration of 20 mg/ml (in 66.6% PEG/water), and administered orally BID (i.e. every 12 hours) to C57BL/6J mice at a dose volume of 3.75 ml/kg. Treatment was started concomitant with the first signs of disease (around days 10-12) and continued for 14 days. The animals were evaluated for general clinical signs, body weight change and neurological score. After termination of treatment the animals were sacrificed, brains and spinal cords were collected, fixed in formalin 4% and processed for staining with haematoxylin and eosin.

Treatment with Compound (2) did show a reduction in the clinical score and better body weight gain of the treated animals as compared to the vehicle group, but the changes were not statistically significant. However, there was a significant reduction of the extent of spinal cord damage in the treatment group versus the untreated control.

The results are illustrated in FIG. 4 which shows the mean extent of spinal cord damage (%) for mice cohorts treated with just the vehicle or Compound (2).

### Kainic Acid Model of Epilepsy

The kainic acid model of temporal lobe epilepsy involves intra-hippocampal injections of kainic acid and presents with neuropathological and electroencephalographic features that are seen in patients with temporal lobe epilepsy, notably the behavioural seizures and neuropathological lesions (Levesque et al. (J. Neuroscience Methods, 260, 45-52 (2016)). Electrodes implanted into the brain are used to monitor seizures; hippocampal neurodegeneration is assessed by immunohistochemistry.

Male NMRI mice (n=21) were given 150 mg/kg of Compound (2) (60% PEG400/water solution) for 3 consecutive days. 2 hours after the last dose the animals were given subcutaneously 45 mg/kg of kainic acid. Behaviour was monitored for 3 hours. On day 7 after receiving kainic acid the animals were sacrificed and brain analysed by immunohistochemistry for markers of neurodegeneration.

The result is that whereas 5 animals (24%) in the untreated control group showed strong neurodegeneration, in the treatment group only 2 animals (9%) exhibited visible signs that they were affected.

1. A compound of formula (I): 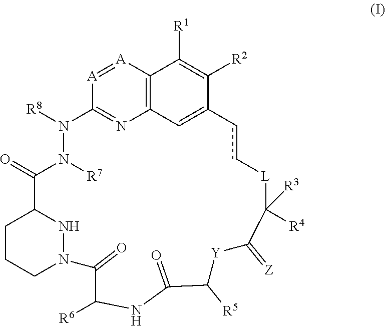
or a salt or solvate thereof, or an optical isomer, enantiomer or diastereoisomer thereof, wherein;
A is independently either C or N;
R¹ and R² are independently H or methyl;
R³ and R⁴ are independently H, C₁₋₇ branched or unbranched alkyl, or can form 4-7 membered carbocyclic or heterocyclic ring in which ring heteroatoms may be O or N, optionally wherein when the heteroatom is N, N is substituted by C₁₋₇ branched or unbranched alkyl or C₁₋₄ branched or unbranched acyl, optionally wherein R³ and R⁴ are independently substituted on any C by H or phenyl;
R⁵ is H, unbranched or branched C₁₋₇-alkyl, a 4-7 membered cyclic or heterocyclic, optionally substituted by carboxy-C₅₋₇-aryl, C₅₋₇-arylamide, C₅₋₇-aryl, 5-7 membered heterocyclic in which the ring heteroatoms may be O or N, C₅₋₇-arylamido, phenyl, 5-7-membered heteroaryl, C₅₋₇-aryl aminosulfonyl, halogen, amido, or amino;
R⁶ is H, unbranched or branched C₁₋₇-alkyl or C₃₋₇-cycloalkyl, or an amido, or a carbonyl, optionally substituted by an amino, carbonyl, 5-7 membered heterocyclic or heteroaryl, or C₅₋₇-aryl;
R⁷ is H, branched or unbranched C₁₋₆-alkyl, or forms a 4-7 membered heterocyclic ring with R⁸;
R⁸ is H, branched or unbranched C₁₋₆-alkyl, acetyl, or forms a 4-7 membered heterocyclic ring with R⁷, optionally substituted by hydroxy, C₅₋₇-aryl, or carbonyl;
the dashed line is either absent or represents a single bond;
L is CH₂ or a bond;
Y is CH₂, NH, N—CH₃ or O;
C═Z is C═O or CH₂.

2. A compound according to claim 1, wherein when the dashed line represents a single bond, the compound adopts a trans configuration about a resulting double bond.

3. A compound according to claim 1, wherein R¹ and R² are H.

4. A compound according to claim 1, wherein R³ is methyl and R⁴ is methyl, benzyl, or methoxymethyl, or wherein R⁴ is methyl and R³ is methyl, benzyl, or methoxymethyl.

5. A compound according to claim 1, wherein R³ and R⁴ form together a piperidine ring, a 1,3-dioxane ring, a cyclobutyl ring, cyclopentyl ring, or a cyclohexyl ring.

6. A compound according to claim 1, wherein R⁵ is H, ethyl, n-propyl, isopropyl, benzyl, chlorobenzyl, methoxybenzyl, 6-(2-aminopyridyl), 3-aminopropyl, chloromethyl, beta-aminocarbonylethyl, 3-piperidinomethyl, or 4-piperidinomethyl, or hexahydropyranyl-4.

7. A compound according to claim 1, wherein R⁷ and R⁸ are joined together to form a 4 to 6 membered heterocyclic ring.

8. A compound according to claim 1, wherein R⁸ is selected from the group consisting of methyl, ethyl, benzyl, acetyl and 3-hydroxypropyl.

9. A compound according to claim 1, wherein R⁶ is 4-(β-naphthyl)-aminobutyl, β-carboxyethyl, β-aminocarbonylethyl, aminocarbonylmethyl, benzyl, or pyridyl.

10. A compound according to claim 1, wherein L is a bond.

11. A pharmaceutical composition comprising a compound of formula (I) as claimed in claim 1, or a salt or solvate thereof, or an optical isomer, enantiomer, or diastereoisomer thereof, and a pharmaceutically acceptable carrier.

12. (canceled)

13. A method of treating or preventing a disease or disorder selected from: neurodegeneration; Alzheimer's disease; Parkinson's disease; X-linked adrenoleukodystrophy; epilepsy; amyotrophic lateral sclerosis (ALS); multiple sclerosis; bipolar disorder; stroke; hearing loss; diseases caused by cellular necrosis and inflammation; acute pancreatitis; surgery-associated acute kidney injury; acute kidney injury caused by toxic drugs; hearing loss caused by ototoxic drugs; acute kidney injury caused by aminoglycoside antibiotics; bacterial sepsis with an antibiotic; sepsis without an antibiotic; diseases wherein the subject has experienced or is suffering from physical trauma or crush injury, exposure to electrical current, extreme physical exertion or activity, and temperature extremes associated with or at risk for onset of rhabdomyolysis; diseases wherein the subject has a pre-existing condition or disease that increases the subject's risk of developing a kidney condition or disease when exposed to a nephrotoxin; diseases where a pre-existing condition or disease increases the risk of chronic kidney disease optionally wherein there is a history of renal impairment or a requirement for dialysis; diseases associated with reduced blood flow; cognitive dysfunction associated with surgery or haemodialysis; or cardiac stunning associated with haemodialysis, wherein the method comprising administering a compound of formula (I) as claimed in claim 1, or a salt or solvate thereof, or an optical isomer, enantiomer, or diastereoisomer thereof, to a patient in need thereof.

14. A method of protecting a transplant organ from ischaemia and inflammation comprising administering a compound of formula (I) as claimed in claim 1, or a salt or solvate thereof, or an optical isomer, enantiomer, or diastereoisomer thereof, to the transplant organ.

15. A method according to claim 14, wherein the compound of formula (I) is administered to an organ donor prior to removal of the organ.

16. A method according to claim 14, wherein the transplant organs is perfused and stored using a solution containing a 0.01-100, preferably 0.01-50, more preferably 0.01-10 micromolar concentration of the compound.

17. A compound according to claim 2, wherein R¹ and R² are H.

18. A compound according to claim 2, wherein R³ is methyl and R⁴ is methyl, benzyl, or methoxymethyl, or wherein R⁴ is methyl and R³ is methyl, benzyl, or methoxymethyl.

19. A compound according to claim 17, wherein R³ is methyl and R⁴ is methyl, benzyl, or methoxymethyl, or wherein R⁴ is methyl and R³ is methyl, benzyl, or methoxymethyl.

20. A compound according to claim 2, wherein R⁵ is H, ethyl, n-propyl, isopropyl, benzyl, chlorobenzyl, methoxybenzyl, 6-(2-aminopyridyl), 3-aminopropyl, chloromethyl, beta-aminocarbonylethyl, 3-piperidinomethyl, or 4-piperidinomethyl, or hexahydropyranyl-4.

21. A compound according to claim 17, wherein R⁵ is H, ethyl, n-propyl, isopropyl, benzyl, chlorobenzyl, methoxybenzyl, 6-(2-aminopyridyl), 3-aminopropyl, chloromethyl, beta-aminocarbonylethyl, 3-piperidinomethyl, or 4-piperidinomethyl, or hexahydropyranyl-4.

22. A compound according to claim 19, wherein R⁵ is H, ethyl, n-propyl, isopropyl, benzyl, chlorobenzyl, methoxybenzyl, 6-(2-aminopyridyl), 3-aminopropyl, chloromethyl, beta-aminocarbonylethyl, 3-piperidinomethyl, or 4-piperidinomethyl, or hexahydropyranyl-4.

23. A compound according to claim 1, wherein:
the dashed line represents a single bond and the compound adopts a trans configuration about a resulting double bond;
R¹ and R² are H;
R³ is methyl and R⁴ is methyl, benzyl, or methoxymethyl, or wherein R⁴ is methyl and R³ is methyl, benzyl, or methoxymethyl;
R⁵ is H, ethyl, n-propyl, isopropyl, benzyl, chlorobenzyl, methoxybenzyl, 6-(2-aminopyridyl), 3-aminopropyl, chloromethyl, beta-aminocarbonylethyl, 3-piperidinomethyl, or 4-piperidinomethyl, or hexahydropyranyl-4;
R⁸ is selected from the group consisting of methyl, ethyl, benzyl, acetyl and 3-hydroxypropyl; and, L is a bond.

24. A compound as claimed in claim 1, wherein the compound is selected from compound number (1) to (82), or a salt or solvate thereof, or an optical isomer, enantiomer or diastereoisomer thereof:

| | Compound |
|---|---|
| Structure | number |
| 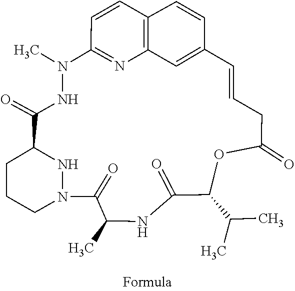 [SMILES: CC(C)[C@H]1OC(=O)C/C=C/c2ccc3ccc(nc3c2)N(C)NC(=O)[C@@H]2CCCN(N2)C(=O)[C@H](C)NC1=O] | 1 |
| 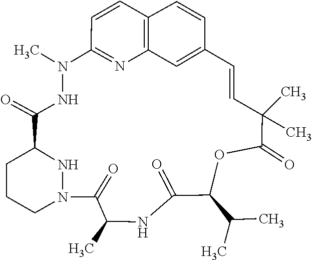 [SMILES: CC(C)[C@@H]1OC(=O)C(C)(C)/C=C/c2ccc3ccc(nc3c2)N(C)NC(=O)[C@@H]2CCCN(N2)C(=O)[C@H](C)NC1=O] | 2 |
| 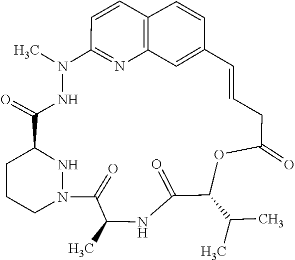 [SMILES: CC(C)[C@H]1OC(=O)C/C=C/c2ccc3ccc(nc3c2)N(C)NC(=O)[C@@H]2CCCN(N2)C(=O)[C@H](C)NC1=O] | 3 |
| 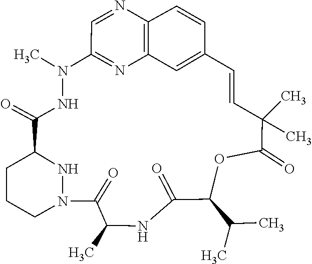 [SMILES: CC(C)[C@@H]1OC(=O)C(C)(C)/C=C/c2ccc3ncc(nc3c2)N(C)NC(=O)[C@@H]2CCCN(N2)C(=O)[C@H](C)NC1=O] | 4 |
| 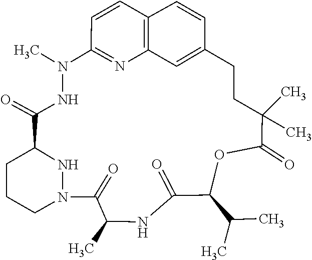 [SMILES: CC(C)[C@@H]1OC(=O)C(C)(C)CCc2ccc3ccc(nc3c2)N(C)NC(=O)[C@@H]2CCCN(N2)C(=O)[C@H](C)NC1=O] | 5 |
| 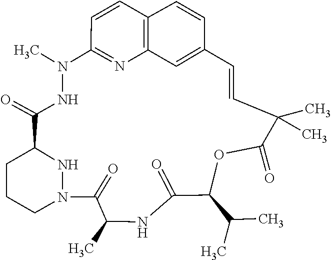 [SMILES: CC(C)[C@@H]1OC(=O)C(C)(C)/C=C/c2ccc3ccc(nc3c2)N(C)NC(=O)[C@@H]2CCCN(N2)C(=O)[C@H](C)NC1=O] | 6 |
| 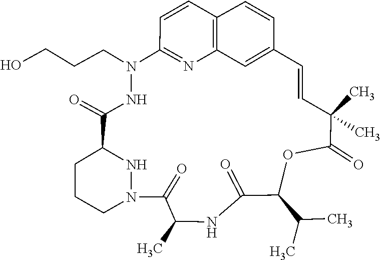 | 7 |
| 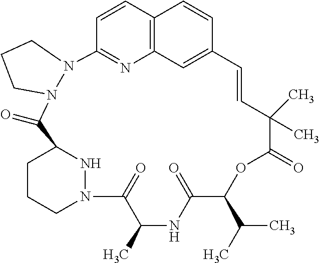 [SMILES: CC(C)[C@@H]1OC(=O)C(C)(C)/C=C/c2ccc3ccc(nc3c2)N2CCCN2C(=O)[C@@H]2CCCN(N2)C(=O)[C@H](C)NC1=O] | 8 |
| 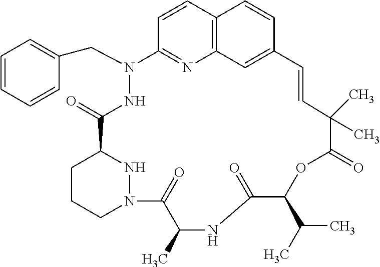 [SMILES: CC(C)[C@@H]1OC(=O)C(C)(C)/C=C/c2ccc3ccc(nc3c2)N(Cc2ccccc2)NC(=O)[C@@H]2CCCN(N2)C(=O)[C@H](C)NC1=O] | 9 |
| 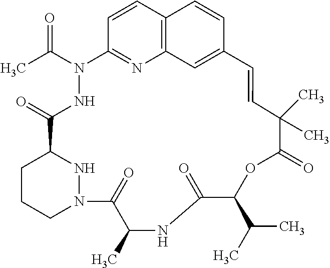 [SMILES: CC(=O)N1NC(=O)[C@@H]2CCCN(N2)C(=O)[C@H](C)NC(=O)[C@H](C(C)C)OC(=O)C(C)(C)/C=C/c2ccc3ccc1nc3c2] | 10 |
| 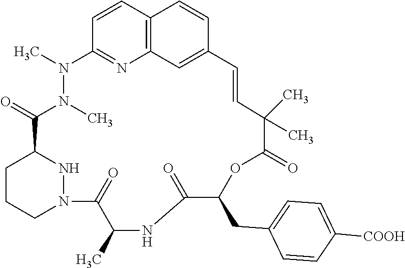 [SMILES: C[C@@H]1NC(=O)[C@H](Cc2ccc(C(=O)O)cc2)OC(=O)C(C)(C)/C=C/c2ccc3ccc(nc3c2)N(C)N(C)C(=O)[C@@H]2CCCN(N2)C1=O] | 11 |
| 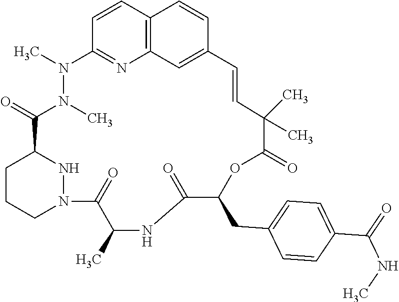 [SMILES: CNC(=O)c1ccc(C[C@@H]2OC(=O)C(C)(C)/C=C/c3ccc4ccc(nc4c3)N(C)N(C)C(=O)[C@@H]3CCCN(N3)C(=O)[C@H](C)NC2=O)cc1] | 12 |
| 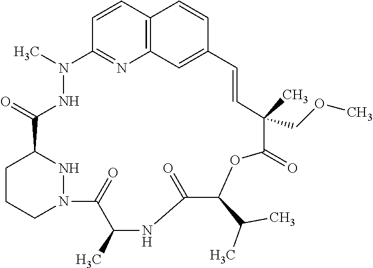 [SMILES: COC[C@@]1(C)/C=C/c2ccc3ccc(nc3c2)N(C)NC(=O)[C@@H]2CCCN(N2)C(=O)[C@H](C)NC(=O)[C@H](C(C)C)OC1=O] | 13 |
| 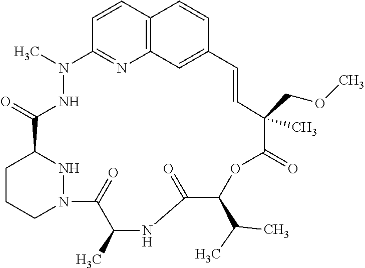 [SMILES: COC[C@]1(C)/C=C/c2ccc3ccc(nc3c2)N(C)NC(=O)[C@@H]2CCCN(N2)C(=O)[C@H](C)NC(=O)[C@H](C(C)C)OC1=O] | 14 |
| 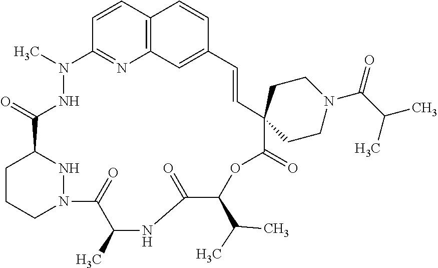 | 15 |
| 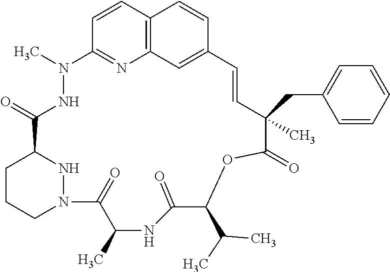 [SMILES: CC(C)[C@@H]1OC(=O)[C@](C)(Cc2ccccc2)/C=C/c2ccc3ccc(nc3c2)N(C)NC(=O)[C@@H]2CCCN(N2)C(=O)[C@H](C)NC1=O] | 16 |
| 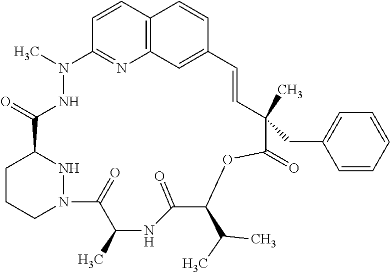 [SMILES: CC(C)[C@@H]1OC(=O)[C@@](C)(Cc2ccccc2)/C=C/c2ccc3ccc(nc3c2)N(C)NC(=O)[C@@H]2CCCN(N2)C(=O)[C@H](C)NC1=O] | 17 |
| 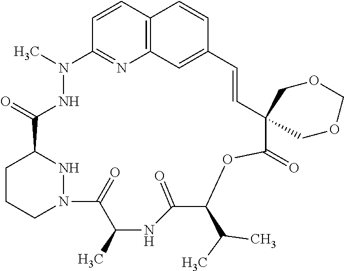 | 18 |
| 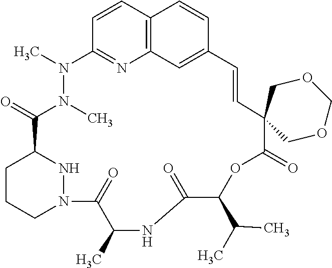 | 19 |
| 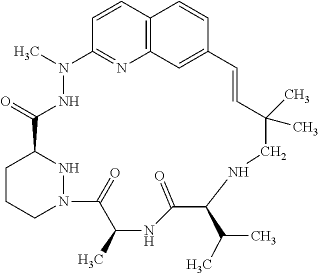 [SMILES: CC(C)[C@@H]1NCC(C)(C)/C=C/c2ccc3ccc(nc3c2)N(C)NC(=O)[C@@H]2CCCN(N2)C(=O)[C@H](C)NC1=O] | 20 |
| 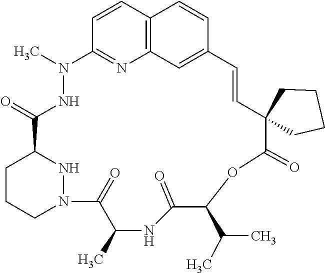 | 21 |
| 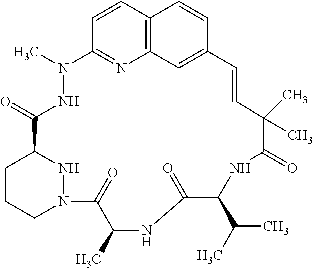 [SMILES: CC(C)[C@@H]1NC(=O)C(C)(C)/C=C/c2ccc3ccc(nc3c2)N(C)NC(=O)[C@@H]2CCCN(N2)C(=O)[C@H](C)NC1=O] | 22 |
| 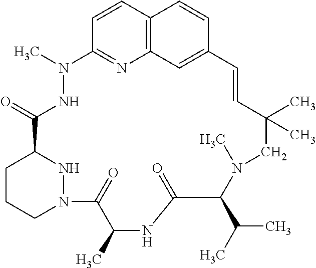 [SMILES: CC(C)[C@H]1C(=O)N[C@@H](C)C(=O)N2CCC[C@H](N2)C(=O)NN(C)c2ccc3ccc(cc3n2)/C=C/C(C)(C)CN1C] | 23 |
| 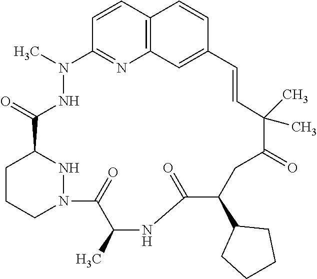 [SMILES: C[C@@H]1NC(=O)[C@H](C2CCCC2)CC(=O)C(C)(C)/C=C/c2ccc3ccc(nc3c2)N(C)NC(=O)[C@@H]2CCCN(N2)C1=O] | 24 |
| 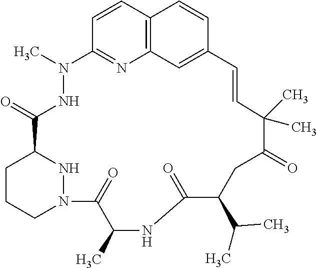 [SMILES: CC(C)[C@@H]1CC(=O)C(C)(C)/C=C/c2ccc3ccc(nc3c2)N(C)NC(=O)[C@@H]2CCCN(N2)C(=O)[C@H](C)NC1=O] | 25 |
| 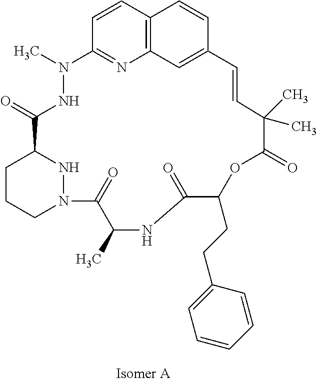 [SMILES: C[C@@H]1NC(=O)C(CCc2ccccc2)OC(=O)C(C)(C)/C=C/c2ccc3ccc(nc3c2)N(C)NC(=O)[C@@H]2CCCN(N2)C1=O] | 26 |
| 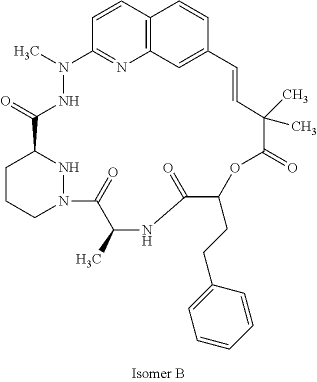 [SMILES: C[C@@H]1NC(=O)C(CCc2ccccc2)OC(=O)C(C)(C)/C=C/c2ccc3ccc(nc3c2)N(C)NC(=O)[C@@H]2CCCN(N2)C1=O] | 27 |
| 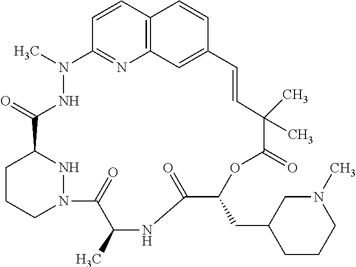 [SMILES: C[C@@H]1NC(=O)[C@@H](CC2CCCN(C)C2)OC(=O)C(C)(C)/C=C/c2ccc3ccc(nc3c2)N(C)NC(=O)[C@@H]2CCCN(N2)C1=O] | 28 |
| 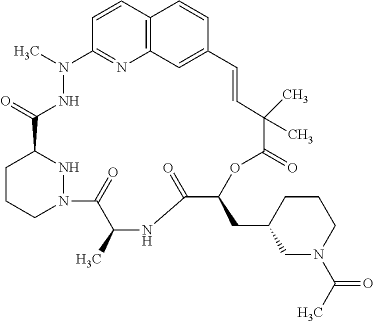 [SMILES: CC(=O)N1CCC[C@@H](C[C@@H]2OC(=O)C(C)(C)/C=C/c3ccc4ccc(nc4c3)N(C)NC(=O)[C@@H]3CCCN(N3)C(=O)[C@H](C)NC2=O)C1] | 29 |
| 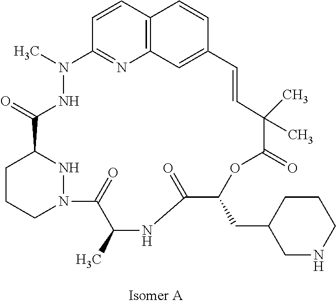 [SMILES: C[C@@H]1NC(=O)[C@@H](CC2CCCNC2)OC(=O)C(C)(C)/C=C/c2ccc3ccc(nc3c2)N(C)NC(=O)[C@@H]2CCCN(N2)C1=O] | 30 |
| 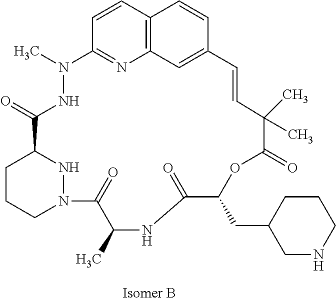 [SMILES: C[C@@H]1NC(=O)[C@@H](CC2CCCNC2)OC(=O)C(C)(C)/C=C/c2ccc3ccc(nc3c2)N(C)NC(=O)[C@@H]2CCCN(N2)C1=O] | 31 |
| 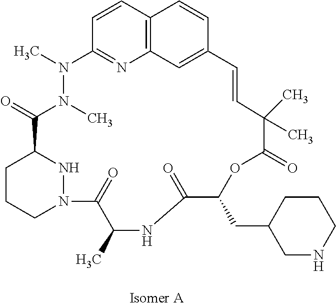 [SMILES: C[C@@H]1NC(=O)[C@@H](CC2CCCNC2)OC(=O)C(C)(C)/C=C/c2ccc3ccc(nc3c2)N(C)N(C)C(=O)[C@@H]2CCCN(N2)C1=O] | 32 |
| 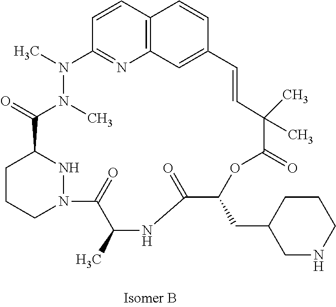 [SMILES: C[C@@H]1NC(=O)[C@@H](CC2CCCNC2)OC(=O)C(C)(C)/C=C/c2ccc3ccc(nc3c2)N(C)N(C)C(=O)[C@@H]2CCCN(N2)C1=O] | 33 |
| 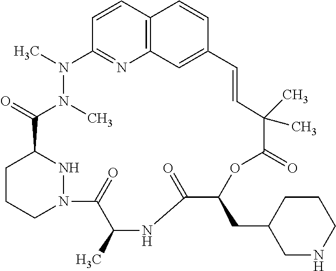 [SMILES: C[C@@H]1NC(=O)[C@H](CC2CCCNC2)OC(=O)C(C)(C)/C=C/c2ccc3ccc(nc3c2)N(C)N(C)C(=O)[C@@H]2CCCN(N2)C1=O] | 34 |
| 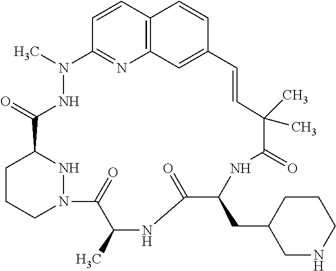 [SMILES: C[C@@H]1NC(=O)[C@H](CC2CCCNC2)NC(=O)C(C)(C)/C=C/c2ccc3ccc(nc3c2)N(C)NC(=O)[C@@H]2CCCN(N2)C1=O] | 35 |
| 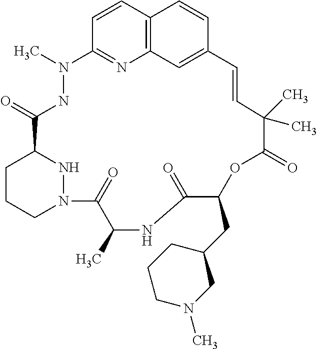 [SMILES: C[C@@H]1NC(=O)[C@H](C[C@@H]2CCCN(C)C2)OC(=O)C(C)(C)/C=C/c2ccc3ccc(nc3c2)N(C)NC(=O)[C@@H]2CCCN(N2)C1=O] | 36 |
| 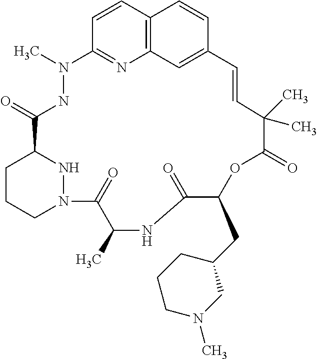 [SMILES: C[C@@H]1NC(=O)[C@H](C[C@H]2CCCN(C)C2)OC(=O)C(C)(C)/C=C/c2ccc3ccc(nc3c2)N(C)NC(=O)[C@@H]2CCCN(N2)C1=O] | 37 |
| 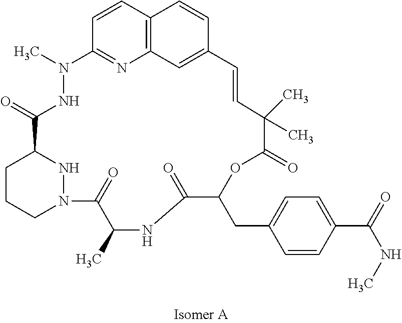 [SMILES: CNC(=O)c1ccc(CC2OC(=O)C(C)(C)/C=C/c3ccc4ccc(nc4c3)N(C)NC(=O)[C@@H]3CCCN(N3)C(=O)[C@H](C)NC2=O)cc1] | 38 |
| 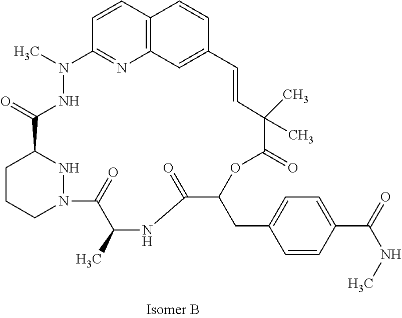 [SMILES: CNC(=O)c1ccc(CC2OC(=O)C(C)(C)/C=C/c3ccc4ccc(nc4c3)N(C)NC(=O)[C@@H]3CCCN(N3)C(=O)[C@H](C)NC2=O)cc1] | 39 |
| 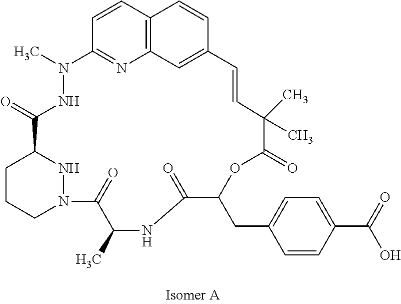 [SMILES: C[C@@H]1NC(=O)C(Cc2ccc(C(=O)O)cc2)OC(=O)C(C)(C)/C=C/c2ccc3ccc(nc3c2)N(C)NC(=O)[C@@H]2CCCN(N2)C1=O] | 40 |
| 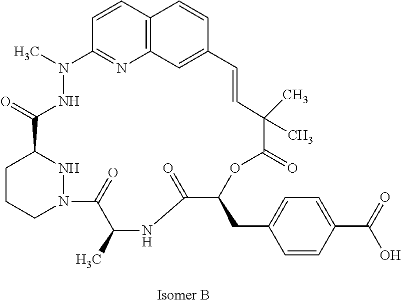 [SMILES: C[C@@H]1NC(=O)[C@H](Cc2ccc(C(=O)O)cc2)OC(=O)C(C)(C)/C=C/c2ccc3ccc(nc3c2)N(C)NC(=O)[C@@H]2CCCN(N2)C1=O] | 41 |
| 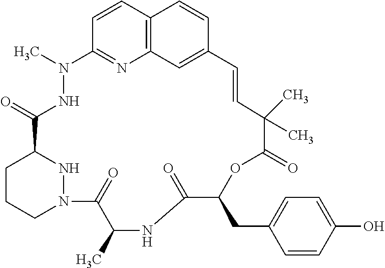 [SMILES: C[C@@H]1NC(=O)[C@H](Cc2ccc(O)cc2)OC(=O)C(C)(C)/C=C/c2ccc3ccc(nc3c2)N(C)NC(=O)[C@@H]2CCCN(N2)C1=O] | 42 |
| 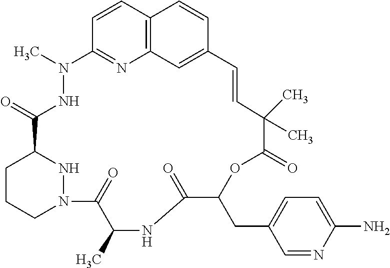 [SMILES: C[C@@H]1NC(=O)C(Cc2ccc(N)nc2)OC(=O)C(C)(C)/C=C/c2ccc3ccc(nc3c2)N(C)NC(=O)[C@@H]2CCCN(N2)C1=O] | 43 |
| 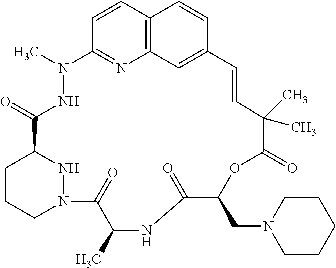 [SMILES: C[C@@H]1NC(=O)[C@H](CN2CCCCC2)OC(=O)C(C)(C)/C=C/c2ccc3ccc(nc3c2)N(C)NC(=O)[C@@H]2CCCN(N2)C1=O] | 44 |
| 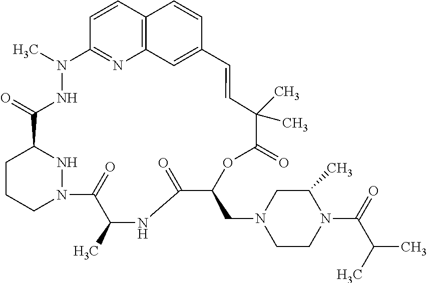 [SMILES: CC(C)C(=O)N1CCN(C[C@@H]2OC(=O)C(C)(C)/C=C/c3ccc4ccc(nc4c3)N(C)NC(=O)[C@@H]3CCCN(N3)C(=O)[C@H](C)NC2=O)C[C@@H]1C] | 45 |
| 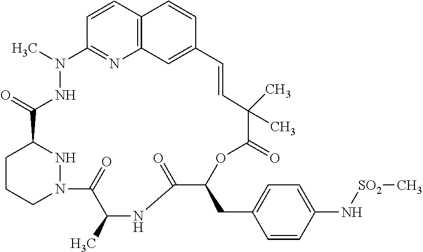 | 46 |
| 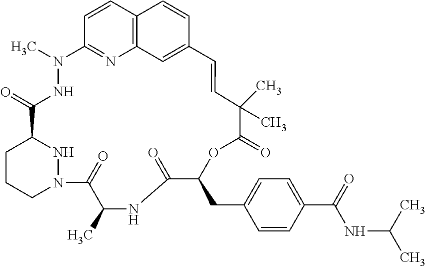 [SMILES: CC(C)NC(=O)c1ccc(C[C@@H]2OC(=O)C(C)(C)/C=C/c3ccc4ccc(nc4c3)N(C)NC(=O)[C@@H]3CCCN(N3)C(=O)[C@H](C)NC2=O)cc1] | 47 |
| 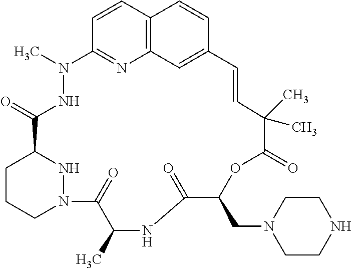 [SMILES: C[C@@H]1NC(=O)[C@H](CN2CCNCC2)OC(=O)C(C)(C)/C=C/c2ccc3ccc(nc3c2)N(C)NC(=O)[C@@H]2CCCN(N2)C1=O] | 48 |
| 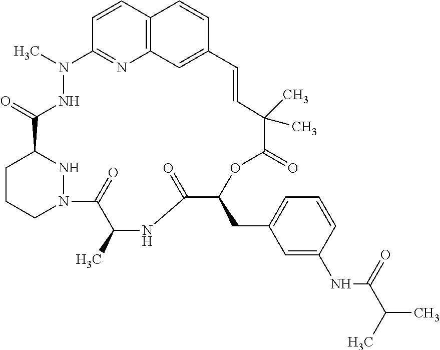 [SMILES: CC(C)C(=O)Nc1cccc(C[C@@H]2OC(=O)C(C)(C)/C=C/c3ccc4ccc(nc4c3)N(C)NC(=O)[C@@H]3CCCN(N3)C(=O)[C@H](C)NC2=O)c1] | 49 |
| 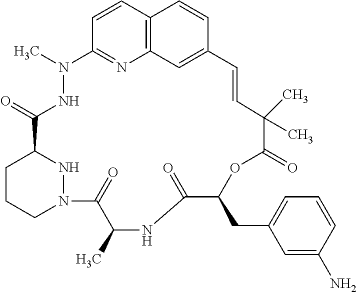 [SMILES: C[C@@H]1NC(=O)[C@H](Cc2cccc(N)c2)OC(=O)C(C)(C)/C=C/c2ccc3ccc(nc3c2)N(C)NC(=O)[C@@H]2CCCN(N2)C1=O] | 50 |
| 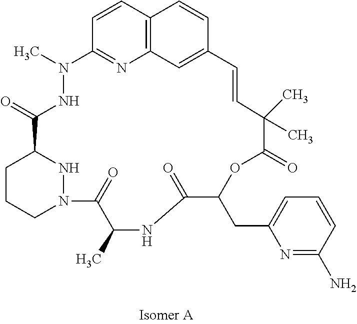 [SMILES: C[C@@H]1NC(=O)C(Cc2cccc(N)n2)OC(=O)C(C)(C)/C=C/c2ccc3ccc(nc3c2)N(C)NC(=O)[C@@H]2CCCN(N2)C1=O] | 51 |
| 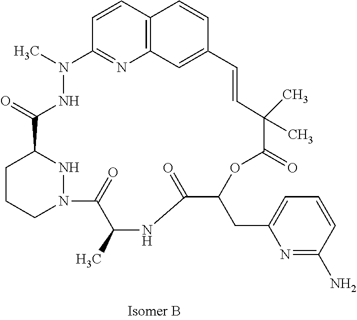 [SMILES: C[C@@H]1NC(=O)C(Cc2cccc(N)n2)OC(=O)C(C)(C)/C=C/c2ccc3ccc(nc3c2)N(C)NC(=O)[C@@H]2CCCN(N2)C1=O] | 52 |
| 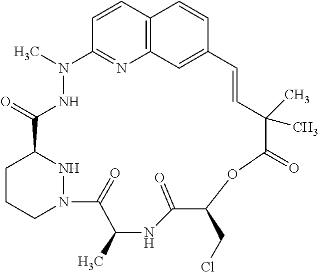 [SMILES: C[C@@H]1NC(=O)[C@H](CCl)OC(=O)C(C)(C)/C=C/c2ccc3ccc(nc3c2)N(C)NC(=O)[C@@H]2CCCN(N2)C1=O] | 53 |
| 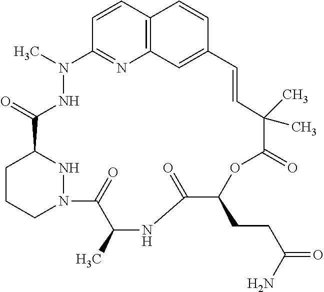 [SMILES: C[C@@H]1NC(=O)[C@H](CCC(N)=O)OC(=O)C(C)(C)/C=C/c2ccc3ccc(nc3c2)N(C)NC(=O)[C@@H]2CCCN(N2)C1=O] | 54 |
| 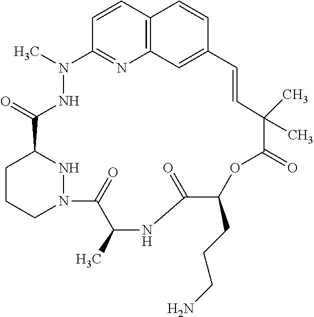 [SMILES: C[C@@H]1NC(=O)[C@H](CCCN)OC(=O)C(C)(C)/C=C/c2ccc3ccc(nc3c2)N(C)NC(=O)[C@@H]2CCCN(N2)C1=O] | 55 |
| 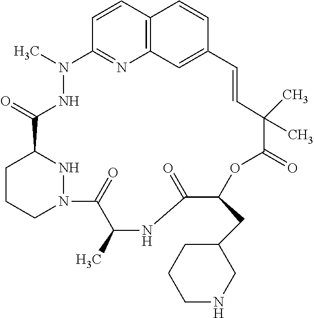 [SMILES: C[C@@H]1NC(=O)[C@H](CC2CCCNC2)OC(=O)C(C)(C)/C=C/c2ccc3ccc(nc3c2)N(C)NC(=O)[C@@H]2CCCN(N2)C1=O] | 56 |
| 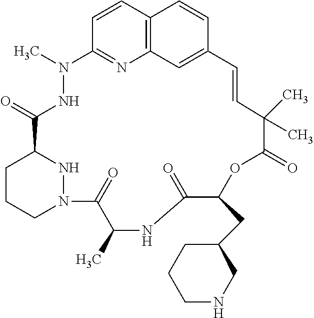 [SMILES: C[C@@H]1NC(=O)[C@H](C[C@@H]2CCCNC2)OC(=O)C(C)(C)/C=C/c2ccc3ccc(nc3c2)N(C)NC(=O)[C@@H]2CCCN(N2)C1=O] | 57 |
| 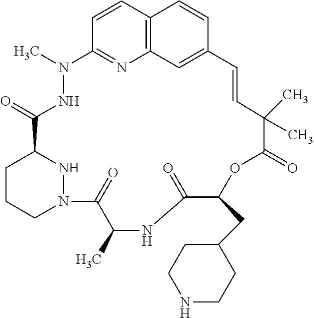 [SMILES: C[C@@H]1NC(=O)[C@H](CC2CCNCC2)OC(=O)C(C)(C)/C=C/c2ccc3ccc(nc3c2)N(C)NC(=O)[C@@H]2CCCN(N2)C1=O] | 58 |
| 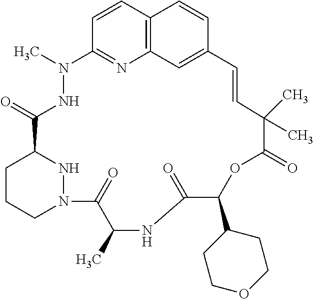 [SMILES: C[C@@H]1NC(=O)[C@H](C2CCOCC2)OC(=O)C(C)(C)/C=C/c2ccc3ccc(nc3c2)N(C)NC(=O)[C@@H]2CCCN(N2)C1=O] | 59 |
| 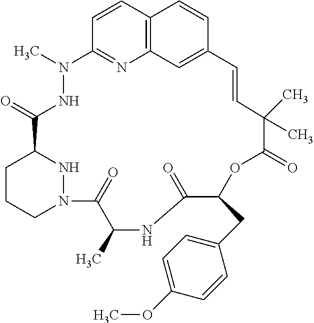 [SMILES: COc1ccc(C[C@@H]2OC(=O)C(C)(C)/C=C/c3ccc4ccc(nc4c3)N(C)NC(=O)[C@@H]3CCCN(N3)C(=O)[C@H](C)NC2=O)cc1] | 60 |
| 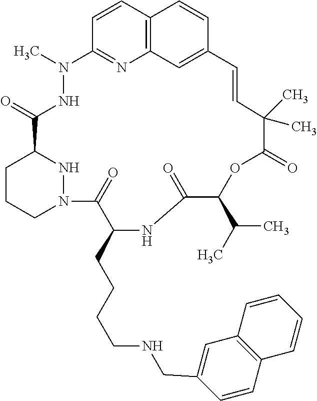 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCCCNCc2ccc3ccccc3c2)NC1=O] | 61 |
| 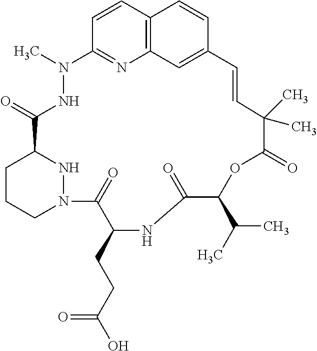 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)O)NC1=O] | 62 |
| 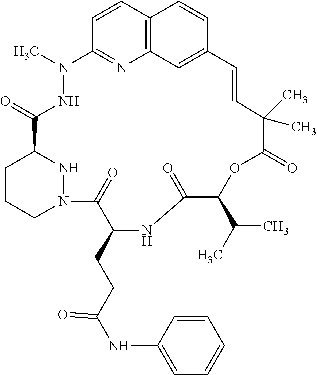 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)Nc2ccccc2)NC1=O] | 63 |
| 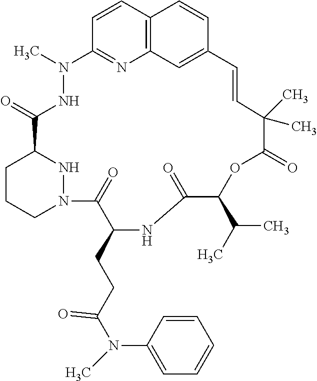 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)N(C)c2ccccc2)NC1=O] | 64 |
| 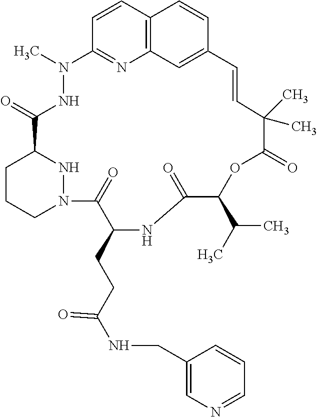 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)NCc2cccnc2)NC1=O] | 65 |
| 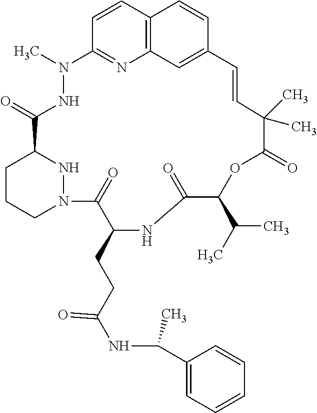 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)N[C@H](C)c2ccccc2)NC1=O] | 66 |
| 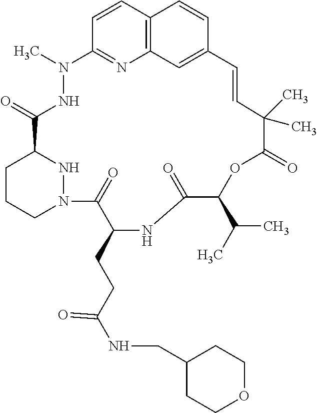 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)NCC2CCOCC2)NC1=O] | 67 |
| 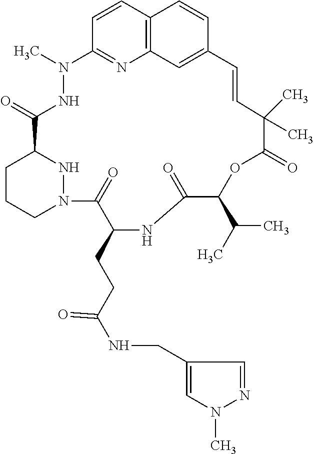 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)NCc2cnn(C)c2)NC1=O] | 68 |
| 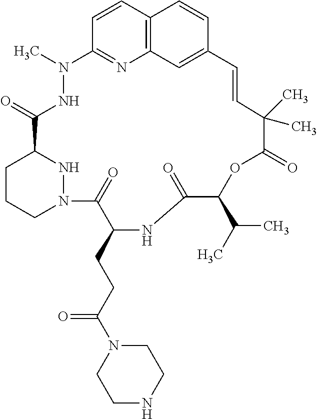 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)N2CCNCC2)NC1=O] | 69 |
| 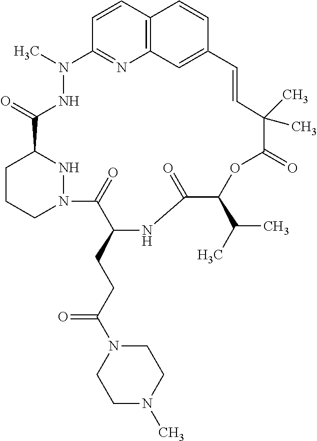 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)N2CCN(C)CC2)NC1=O] | 70 |
| 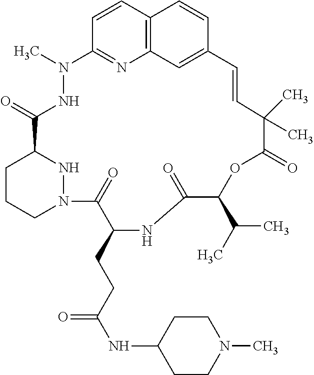 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)NC2CCN(C)CC2)NC1=O] | 71 |
| 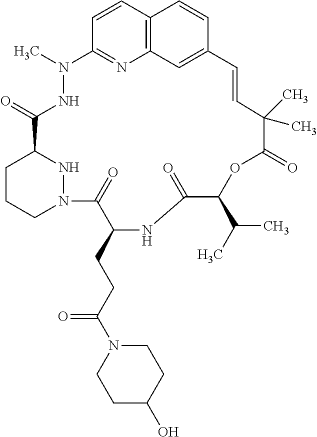 [SMILES: CC(C)[C@@H]1OC(=O)C(C)(C)/C=C/c2ccc3ccc(nc3c2)N(C)NC(=O)[C@@H]2CCCN(N2)C(=O)[C@H](CCC(=O)N2CCC(O)CC2)NC1=O] | 72 |
| 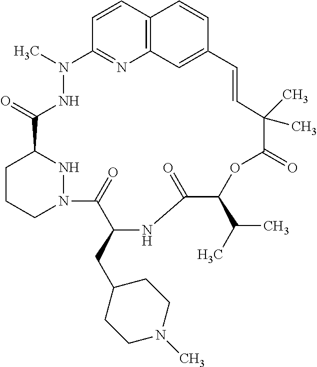 [SMILES: CC(C)[C@@H]1OC(=O)C(C)(C)/C=C/c2ccc3ccc(nc3c2)N(C)NC(=O)[C@@H]2CCCN(N2)C(=O)[C@H](CC2CCN(C)CC2)NC1=O] | 73 |
| 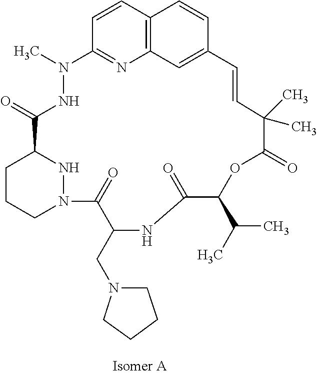 [SMILES: CC(C)[C@@H]1OC(=O)C(C)(C)/C=C/c2ccc3ccc(nc3c2)N(C)NC(=O)[C@@H]2CCCN(N2)C(=O)C(CN2CCCC2)NC1=O] | 74 |
| 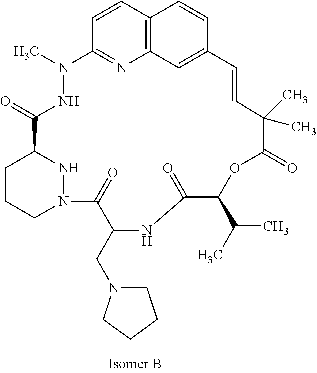 [SMILES: CC(C)[C@@H]1OC(=O)C(C)(C)/C=C/c2ccc3ccc(nc3c2)N(C)NC(=O)[C@@H]2CCCN(N2)C(=O)C(CN2CCCC2)NC1=O] | 75 |
| 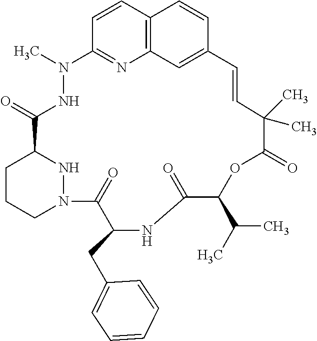 [SMILES: CC(C)[C@@H]1OC(=O)C(C)(C)/C=C/c2ccc3ccc(nc3c2)N(C)NC(=O)[C@@H]2CCCN(N2)C(=O)[C@H](Cc2ccccc2)NC1=O] | 76 |
| 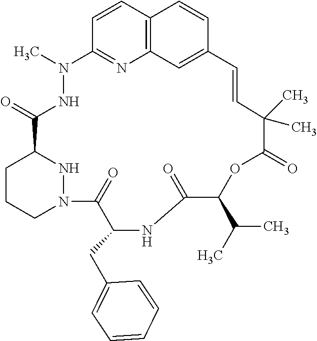 [SMILES: CC(C)[C@@H]1OC(=O)C(C)(C)/C=C/c2ccc3ccc(nc3c2)N(C)NC(=O)[C@@H]2CCCN(N2)C(=O)[C@@H](Cc2ccccc2)NC1=O] | 77 |
| 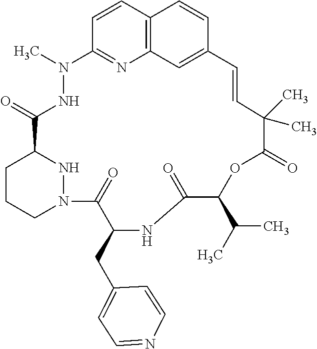 [SMILES: CC(C)[C@@H]1OC(=O)C(C)(C)/C=C/c2ccc3ccc(nc3c2)N(C)NC(=O)[C@@H]2CCCN(N2)C(=O)[C@H](Cc2ccncc2)NC1=O] | 78 |
| 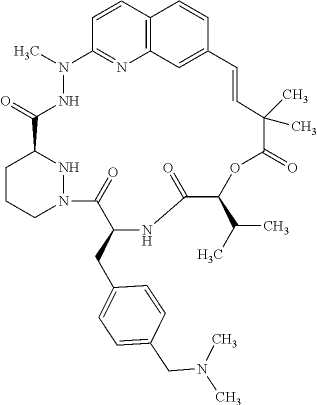 [SMILES: CC(C)[C@@H]1OC(=O)C(C)(C)/C=C/c2ccc3ccc(nc3c2)N(C)NC(=O)[C@@H]2CCCN(N2)C(=O)[C@H](Cc2ccc(CN(C)C)cc2)NC1=O] | 79 |
| 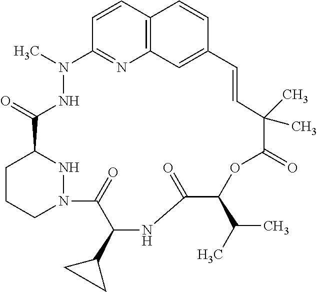 [SMILES: CC(C)[C@@H]1OC(=O)C(C)(C)/C=C/c2ccc3ccc(nc3c2)N(C)NC(=O)[C@@H]2CCCN(N2)C(=O)[C@H](C2CC2)NC1=O] | 80 |
| 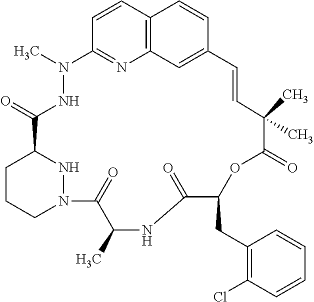 | 81 |
| 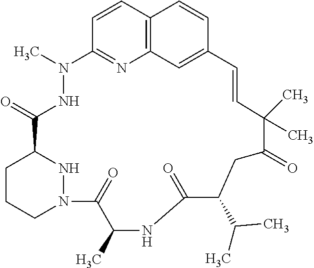 [SMILES: CC(C)[C@H]1CC(=O)C(C)(C)/C=C/c2ccc3ccc(nc3c2)N(C)NC(=O)[C@@H]2CCCN(N2)C(=O)[C@H](C)NC1=O] | 82 |
